(19) United States
(12) Patent Application Publication
(10) Pub. No.: US 2026/0272532 A1
(43) Pub. Date: Sep. 17, 2026

(54) ABLATION TREATMENT DEVICES AND METHODS

(71) Applicant: Pulse Biosciences, Inc., Hayword, CA (US)

(72) Inventors: KEVIN L. MOSS, Tracy, CA (US); Cameron D. HINMAN, Thurmond, NC (US); Ashley L. Oliver, Piedmont, CA (US); Richard L. Nuccitelli, Millbrae, CA (US); Douglas L. Polzin, Castle Pines, CO (US)

(21) Appl. No.: 19/676,633

(22) Filed: May 13, 2026

Related U.S. Application Data
(63) Continuation-in-part of application No. 18/701,211, filed on Sep. 15, 2022.
(60) Provisional application No. 63/271,954, filed on Oct. 26, 2021.

(51) Int. Cl. A61B 18/00

(52) U.S. Cl. CPC A61B 18/00; A61B 2018/00077; A61B 2018/00083; A61B 2018/00178; A61B 2018/00577; A61B 2018/00761; A61B 2018/00767; A61B 2218/002; A61B 2218/007

(57) ABSTRACT

Described herein are treatment tools and methods for applying electric treatment to target tissue. In some examples the treatment tools described herein allow for adjusting the spacing between the proximal electrode(s) and the distal electrode of the treatment tools. The treatment tools described herein may also be configured to preserve strength and resilience to buckling in the treatment tip region of the device. Apparatuses herein may be multimodal, configured to apply an electric treatment to target tissue as well as to aspirate, collect, or otherwise remove material; infuse/deliver material to the target tissue; or both.

### CLAIM OF PRIORITY

This patent application claims priority to, as a continuation-in-part of, co-pending U.S. application Ser. No. 18/701,211, titled “PERCUTANEOUS TREATMENT DEVICE AND METHOD,” published on Jan. 16, 2025 as US2025/0017647A1, which is a 371 U.S. national stage entry of, and claims priority to, PCT/US2022/076507, titled “PERCUTANEOUS TREATMENT DEVICE AND METHOD,” published on May 4, 2023 as WO2023/076767A1, which claims priority to U.S. provisional patent application No. 63/271,954, titled “PERCUTANEOUS TREATMENT DEVICE AND METHOD,” filed on Oct. 26, 2021, each of which is herein incorporated by reference in their entirety.

### BACKGROUND

Various electrical devices, including in the form of radio frequency (RF), microwave, cryo, laser, or pulsed electric energy delivery devices, are commonly used for treating certain conditions and diseases. For example, electric pulses have been described for electromanipulation of biological cells. Electric pulses, including short, high-field strength electric pulses, may be used in treatment of human cells and tissue including benign and malignant tumor cells, lesions, various tissue and skin growth and conditions. Treatments with electric pulses, including higher electric field strengths and shorter electric pulses, may be useful in manipulating intracellular structures, such as nuclei and mitochondria. For example, sub-microsecond (e.g., nanosecond) high voltage pulse generators and treatment applicators have been proposed for biological, medical and cosmetic applications. However, such higher peak electric fields are more likely to arc between the electrodes.

It would be particularly advantageous to be able to treat various tissue and anatomical structures of a subject with electric fields using a variety of approaches as applicable to a variety of procedures, such as, e.g., catheter-based approaches, percutaneous approaches, laparoscopic approaches, and, e.g., endoscopic approach(es), for example, with a percutaneous needle-type apparatus or tool or, e.g., an endoluminal needle-type apparatus or tool. However, delivering therapeutic, high-voltage energy has a substantial risk inducing electrical shock, arcing, burns, or, e.g., internal-organ damage, risks that are even more acute when the high voltage device is intended to be inserted into the body. Because of the complexity of this challenge, no known effective devices have been developed.

Further, conditions wherein tissues and anatomical structures of a subject are treated with electric fields are often accompanied by a need or desire to retrieve target tissue/structure sample(s), deliver a treatment material to the target tissue (such as, e.g., the delivery of a therapeutic material such as, e.g., a chemotherapeutic agent/drug), or both. Such additional intervention(s) requires the performance of one or more separate procedures or, alternatively, requires the exchange of a plurality of devices during a single procedure to facilitate the switching of modalities. It would be advantageous to reduce the time, cost, and risk associated with such adjuvant procedure(s) and to facilitate the conduct of such procedures in a timely and reduced-risk manner concomitantly with the delivery of electric field treatments. However, device(s) designed to deliver therapeutic, high-voltage energy encounter significant design challenges in isolation, such as avoiding electrical shock, arcing, burns, or, e.g., internal-organ damage, especially acute when introduced intracorporeally, as described above. Such challenges have prohibited advancement of such high-energy delivery device(s) themselves, let alone the development of such devices capable of facilitating multi-modal devices, especially in high voltage environment.

It would be beneficial to provide devices, such as treatment apparatuses, and corresponding methods that may apply high voltage, electrical pulses, such as microsecond or sub-microsecond (e.g., nanosecond) electric pulses, to treat patients while mitigating the aforementioned risks. Further, it would be beneficial for such tools to be capable of facilitating the extraction or delivery of material(s) from a treatment site in a timely, cost-effective, and reduced-risk manner.

### SUMMARY OF THE DISCLOSURE

Described herein are apparatuses (e.g., systems, devices and tools, including applicators) and methods for treating various anatomical structures using electrical fields. In general, these apparatuses and methods may be useful for treating a subject by the application of therapeutic energy, including but not limited to short, high field strength electric pulses, such as pulses having microsecond or sub-microsecond (e.g., nanosecond) duration. The systems, devices and methodologies described herein are especially useful with non-thermal pulsed electric fields (e.g., nanosecond pulsed electric fields, etc.), however, in some implementations they may be also used with other energy modalities, including (but not limited to) radio frequency (RF) energy. The devices, systems and methods described herein are also configured to avoid or reduce arcing, especially when applying high voltage electric fields.

The methods and apparatuses (e.g., devices, systems, etc.) described herein may apply microsecond or sub-microsecond (e.g., nanosecond) pulsed electrical fields to treat lesions, tumors, nodules and other growth, diseases and conditions, for example, in a target tissue, including various anatomical structures. Such anatomical structures may include muscular organs (e.g., smooth muscle, cardiac and skeletal muscles), circulatory organs (e.g., heart, arteries, veins), respiratory organs (e.g., lungs), abdomen and digestive organs (e.g., stomach, duodenum, intestines, liver, pancreas), urinary organs (e.g., kidneys, ureters, bladder), immune system organs (e.g., lymph nodes, bone marrow, thymus), nervous system organs (e.g., brain, spinal cord, nerves), endocrine organs (e.g., pituitary gland, thyroid, adrenal glands), reproductive organs (e.g., penis, vagina, prostate, uterus, testicle), skeletal organ (e.g., bones).

In some examples, the apparatuses may be used in various minimally invasive procedures and techniques, including endoluminally (e.g., catheter-based, or through natural body orifices or small incisions). In some examples, the apparatuses, e.g., treatment applicators, described herein may provide easy percutaneous access to a target tissue, such as thyroid, and perform electric field treatments of the affected target tissue, for example, treatment of the thyroid nodule. In various examples, the apparatuses provided herein may facilitate easy endoscopic access to a target tissue, such as an endoluminal target site, and perform electric field treatments of the affected target tissue. In some examples, apparatuses may be multi-modal and capable of providing any one or more of: electric field treatment, delivery of a material such as a therapeutic agent (or, e.g., other fluid) to a target site, or extracting a sample of material from a target site (e.g., biopsy). Exemplary apparatuses disclosed herein may be used in procedures requiring fine needle aspiration (FNA), fine needle biopsy (FNB), or both. The disclosed apparatuses may allow for less trauma to the target tissue and may also allow to adjust the length/depth and size of the area being treated by adjusting a distance between the electrodes of the apparatus. The apparatuses described herein may be used with a variety of different generator systems, for example, microsecond or sub-microsecond, e.g., nanosecond, pulse generators. Disclosed apparatuses may be utilized in procedures accompanied by use of ultrasound, such as, e.g., endoscopic ultrasound, endobronchial ultrasound, etc.

The apparatuses described herein may be configured for manual or automated (e.g., robotic assisted) or semi-automated control and may be particularly well suited for use with various fully and partially automated systems, such as robotic systems. In some variations these apparatuses may be integrated into systems that are configured to be mounted onto or coupled to a movable (e.g., robotic) arm of a robotic system. In some variations instruments may be guided and controlled by the robotic system during a medical or cosmetic procedure.

For example, described herein are apparatuses for delivering an electric field treatment that include: a handle; an elongate shaft extending from the handle; a tip region at a distal end of the elongate shaft, the tip region comprising: a first electrode, a second electrode, and a spacer between the first electrode and the second electrode; and a length adjuster configured to adjust a distance between the first electrode and the second electrode.

In certain aspects, apparatuses for delivering an electric field treatment may comprise one or more components to facilitate the aspiration (e.g., removal, withdrawal, suction) of material from a target location, the delivery (e.g., infusion, expelling) of material to a target location, or both. In some examples, extension of spacing between first and second electrodes exposes a lumen, facilitating the aspiration or delivery of material(s). In other examples, dedicated lumen(s) facilitates the aspiration or delivery of material(s).

In some examples, apparatuses comprise at least two electrodes within a treatment portion of the apparatus, wherein the treatment portion of the apparatus comprises one or more feature(s) or structure(s) providing strength, support, rigidity or otherwise reducing the flexibility of one or more portions of the apparatus.

Any of these apparatuses may be configured to include a vacuum outlet at the distal end (tip region) that may assist in making contact between the tissue and electrodes, assist in the collection of material such as cells/tissue from a target location, or both. For example, the apparatus may include a vacuum channel configured to provide negative pressure at the tip region. Alternatively or additionally, the apparatus may include an infusion channel configured to deliver a solution, such as, a therapeutic composition or material, or other fluid from the tip region. The tip region may include a vacuum outlet or an infusion outlet, e.g., disposed between the first electrode and the second electrode. The tip region may include a vacuum outlet and/or an infusion outlet, e.g., disposed at the distal end (tip) of a distal electrode, such as an electrode that may be configured with a tissue-penetrating distal end. In some examples the same channel may be used for infusion as for vacuum.

The electrodes may be electrically coupled to a pulse generator by one or more connections in the handle. These connections may be configured to prevent leak current (e.g., creepage) within the handle, which may be particularly important when high voltages are used, as may be the case with sub-microsecond pulsing in some treatment regimens. For example, the apparatus may include a first wire connecting to the first electrode and a second wire connecting to the second electrode. These connections may be isolated from each other and from within the handle. For example, in some examples, the handle comprises an insulating baffle configured to provide a minimum clearance distance between electrical contacts for the first electrode and the second electrode within the handle.

In any of these apparatuses the first electrode may be proximal to the second electrode along the tip region. For example, the first electrode may be a circumferential electrode, and the second electrode may be configured with a tissue-penetrating distal end.

The spacer may be conductive or insulative. In some examples it may be particularly beneficial to include a conductive spacer between the first and second (or between first, second and third or more) electrodes. The spacer may have a circumference that is greater than a circumference of either of the first electrode and the second electrode. The circumference may refer to the outer dimension of the spacer or outer radial dimension; in some examples the circumference may refer to the outer diameter (OD); in general, the circumference does not need to be round but may have any shape.

The spacer between the first and second electrode may also be configured to reduce arcing by providing a long minimum clearance distance (e.g., minimum creepage path distance) between the first electrode and the second electrode. For example, the spacer may be coupled to the proximal end of the second (distal) electrode and may be configured so that the minimum clearance distance or path from the second electrode to the first (proximal) electrode is along the spacer. The spacer may also be configured so that the minimum clearance distance/path is greater than the minimum distance between the first electrode and the second electrode (e.g., the distance between the first and second electrodes on the outside of the device). In some examples, the spacer may extend proximally from a proximal end of the second electrode and proximal to the first electrode; the spacer may also extend radially inwards of the first electrode.

In any of the apparatuses described herein the apparatus may include a first elongate member to which the first electrode is coupled, and a second elongate member concentrically within the first elongate member and to which the second electrode is coupled. The first elongate member may form the outer portion of the shaft or may be housed within the shaft. Thus, in some examples, the first elongate member may form at least a portion of the elongate shaft.

The length adjuster may be configured to drive movement (e.g. axial movement in the proximal-to-distal direction) of the first elongate member relative to the second elongate member. For example, the length adjuster may include a threaded body configured to convert rotational movement of an outer portion of the length adjuster into linear movement of the second elongate member relative to the first elongate member to move the second electrode relative to the first electrode to adjust the distance between the first electrode and the second electrode. In some examples the length adjuster comprises an adjuster knob configured to drive movement of a stator coupled to the second elongate member. The adjuster knob may be configured to rotate clockwise or counterclockwise to drive the stator proximally or distally without rotating the stator.

In any of these methods and apparatuses, the distance between the first electrode and the second electrode may be adjustable, for example, from 1 mm to 7 mm, or more, such as 8 mm or more. In any of these examples adjusting the distance between the first and second electrodes may also adjust a spacer between the first and second electrodes. In some examples adjusting the spacing between the first and second electrodes may cause the outer circumference (e.g., the outer diameter) of the spacer to expand or contract; however, in some examples the spacer outer circumference may be adjusted separately from the electrode spacing.

Thus, in any of these apparatuses and methods the spacer may be stretched or compressed, which may reduce or increase the outer circumference (e.g., diameter) of the spacer. In some examples it may be beneficial to reduce arcing to have the spacer have an outer diameter (OD) that is greater than either the OD of the first or second electrodes. However, it may also be beneficial to insert the apparatus into the tissue with a more uniform outer diameter (e.g., low profile circumference) without the spacer having an OD extending further than the ODs of the first or second electrodes. In any of the methods and apparatuses the spacer may be configured so that the circumference (e.g., OD) of the spacer may be reduced (e.g., during insertion) and expanded to a larger circumference (e.g., OD), for example, when the apparatus is in place for the application of energy. For example, the spacer may be formed of an elastomeric material that has an expanded configuration with an OD that is larger than the ODs of either the first or second electrodes. The proximal end of the spacer may be coupled to a member that may pull (and/or push) the spacer to compress it, so that the OD of the spacer is reduced, e.g., for insertion. Alternatively, the spacer may be in a normally lower profile configuration (e.g., which may be the same as or less than the OD of the first and/or second electrode), and the proximal end may be pushed to compress the spacer so that the OD expands to a larger dimension than the OD of both the first and second electrodes.

In some examples of the apparatuses described herein the second electrode comprises a tissue-penetrating end, which may be configured as a trocar, a cone, or a hybrid of a trocar and a cone or other smooth surface shape.

In some examples of the apparatuses described herein, a tip region with one or more electrodes may comprises one or more lumens therethrough. Such lumen(s) may be present in any suitable configuration, e.g., concentrically aligned with one or more other lumens of the apparatus(es). In examples, lumens may be non-concentric, such as, e.g., running parallel to one another. In a particular example of the apparatuses described herein, the second electrode may comprise a tissue-penetrating end and at least one lumen extending through the electrode, wherein the distal end of at least one of the one or more lumens is positioned at or near the tissue-penetrating end of the electrode.

Any of the apparatuses described herein may include a pulse generator configured to generate a plurality of electrical pulses having amplitude of at least 0.1 kV and a duration of less than 1000 nanoseconds. Such pulse generator may be configured to generate high voltage electrical pulses, including for example, pulses of at least 0.5 kV, 1 kV, 2 kV, 3 kV, 4 kV, 5 kV, 6 kV, 7 kV, 8 kV, 9 kV, 10 kV, 11 kV, 12 kV, 13 kV, 14 kV, 15 kV, 16 kV, 17 kV, or, e.g., at least 18 kV or more.

In any of these apparatuses described herein, each of the first electrode and the second electrode may include a curved edge (referred to herein as a fillet) on each side of the first electrode and the second electrode facing the spacer.

For example, an apparatus for delivering a pulsed electric field may include: a handle; an elongate shaft extending from the handle; a tip region extending distally from the elongate shaft, the tip region comprising: a first electrode, a second electrode, and a conductive spacer between the first electrode and the second electrode.

Thus, an apparatus for delivering a high voltage electric field may be configured to reduce peak electric field. The apparatus may comprise a handle and a tip region coupled to the handle, the tip region comprising a first electrode, a second electrode, and a spacer between the first electrode and the second electrode, wherein the first electrode comprises at least one first fillet on a first side adjacent to the spacer and the second electrode comprises at least one second fillet on a second side adjacent to the spacer such that the at least one first fillet and the at least one second fillet are configured to reduce or eliminate arcing between the first and the second electrode. In some examples, the fillets are configured to reduce peak electric field by, for example, about 10% to about 30%. The spacer between the electrodes may be insulating or in some examples it may be a conductive spacer. In some examples the spacer may be a balloon. Also, in some examples, the spacer may have a circumference (e.g. diameter) the same or greater than a circumference of the electrodes. In further examples, the circumference of the spacer near the middle of the spacer may be greater than the circumferences of the electrodes while each end of the spacer adjacent to the electrodes may have the same circumference as the electrodes, and the circumference of the spacer may gradually reduce in the direction from the middle of the spacer towards each end. The apparatus may further comprise a length adjuster configured to adjust distance between the first electrode and the second electrode. In some examples, one of the electrodes that is at a distal end of the tip region may have a trocar configuration, a conical configuration (“cone” or “pencil”), or a hybrid configuration. In some embodiments, the electrodes may be bipolar, which in other embodiments the electrodes may be monopolar.

For example, an apparatus for delivering a pulsed electric field may include: a handle; an elongate shaft extending from the handle; and a tip region extending distally from the elongate shaft, the tip region comprising: a first electrode, a second electrode, and a spacer between the first electrode and the second electrode, wherein the first electrode comprises a first rounded edge on a first side of the first electrode that is adjacent to the spacer and the second electrode comprises a second rounded edge on a second side of the second electrode adjacent to the spacer, wherein the first rounded edge and the second rounded edge are configured to reduce or eliminate arcing between the first electrode and the second electrode.

The apparatus for delivering a pulsed electric field may comprise a handle and a tip region coupled to the handle, wherein the tip region comprises a first electrode, a second electrode, and a conductive spacer between the first electrode and the second electrode.

The circumference (e.g., outer circumference) of the conductive spacer may be same as the circumference of each of the first electrode and the second electrode. In some examples, the circumference of the conductive spacer may be greater than a circumference of the electrodes. For example, the circumference of the conductive spacer near a middle portion of the conductive spacer may be greater than a circumference of the first electrode and the second electrode, and each end of the conductive spacer adjacent to the first electrode and the second electrode may have the same circumference as the circumference of the first electrode and the second electrode, further wherein the circumference of the conductive spacer tapers from a middle of the conductive spacer towards each end of the conductive spacer. The circumference of the conductive spacer may be adjusted depending on the shape of the target region and/or the treatment requirement of the applied electric field.

In some examples, the electrodes adjacent to the conductive spacer may have fillets (rounded corners). In some examples, the second electrode is at a distal end of the tip region, and this distal electrode may have a trocar configuration, a conical configuration, or a hybrid configuration.

The circumference of the conductive spacer may be adjusted depending on the shape of the target region and/or the treatment requirement of the applied electric field.

In some embodiments, the conductive spacer may be (or may include) a hydrogel, a conductive adhesive, a conductive gel, a conductive silicone, a urethane rubber, conductive thermoset, thermoplastic resins, any other biocompatible material with the desired conductivity, any semi-conductor material, or any combination thereof.

In some embodiments, a conductivity of the conductive spacer is substantially equal to ten times (10×) a conductivity of a tissue of the treatment area. In some embodiments, a conductivity of the conductive spacer is greater than or equal to a conductivity of a tissue of the treatment area and less than or equal to one hundred times (100×) the conductivity of the tissue of the treatment area.

The present disclosure is also directed to methods of using any of the apparatuses described herein, including selecting an electrode assembly. In some examples, selecting the electrode assembly may be based on the conductive spacer having a conductivity that is less than, greater than, or equal to a conductivity of a tissue of the treatment area. In further examples, the method may include selecting the electrode assembly based at least in part on a conductivity of the conductive spacer. Also, selecting the electrode assembly may be further based at least in part on a size of the electrode assembly and a size of the treatment area. In some examples, the method of treating a tissue with a pulsed electric field may include selecting the voltage to be applied to the treatment area based at least in part on a conductivity of the conductive spacer.

In some embodiments, the conductive spacer can reduce the peak electric field by 25%-50% compared to the electrodes with the insulative spacer. The conductive spacer may have another advantage of providing more uniform treatment between the electrodes by strengthening electric field in the middle between the electrodes of the tip region.

In some examples the apparatus for delivering electric treatment includes a handle and a tip region coupled to the handle, the tip region comprising a first electrode, a second electrode, and a spacer between the first electrode and the second electrode, wherein a maximum circumference of the spacer is greater than a maximum circumference of the first electrode and the second electrode.

As mentioned above, in some examples the distal end of the tips described herein may be configured as an electrode having a tissue-penetrating shape. The shape may be a cone (e.g. pencil) shape, or in some examples a trocar shape, having multiple cutting edges. In some examples a hybrid of the cone and trocar shapes may be used. For example, an apparatus for delivering electric treatment may include a tip region extending distally from the elongate shaft, the tip region comprising: a first electrode, a second electrode distal to the first electrode, wherein the second electrode comprises a tissue-penetrating distal end configured as a hybrid of a cone and a trocar; and a spacer between the first electrode and the second electrode. For example, the second electrode may include a distal trocar region having three or more blade edges extending proximally from a distal end and a proximal conical region having a smooth conical face extending proximally from a proximal end of each blade. The change in angle between the three or more blade edges and the smooth conical face may be less than 15 degrees (e.g., less than about 15 degrees, less than about 14 degrees, less than about 13 degrees, less than about 12 degrees, less than about 11 degrees, less than about 10 degrees, etc.).

In some examples, the tip region may have any combination of the features described herein, including any of the spacers, electrode fillets (rounded edges) and any configuration of the distal electrode (e.g., cone, trocar, hybrid), as well as vacuum and/or infusion outlets (if applicable) and length adjustment capabilities. For example, in some embodiments, the tip region may have a trocar tip and a conductive spacer with the same circumference as electrodes. In another example, the tip region may have a cone/pencil tip and a conductive spacer with the same circumference as electrodes. This type of tip region may mitigate arcing by both decreasing the peak electric field and also forcing the tissue to stretch during insertion improving the contact with the tissue. Also, in any of the examples of the present disclosure, the apparatus may be configured to provide an effective minimum clearance distance to avoid or reduce arcing without increasing the actual physical distance between the electrodes.

As mentioned above, in any of the apparatuses and methods described herein the spacer may extend proximally to the proximal (e.g., first) electrode, which may increase the minimum clearance/creepage distance between the first and second (e.g., distal) electrodes. The proximal electrode, and at least part of the insulated first electrical connector connecting the proximal electrode to the pulse generator, may be coaxially arranged over the insulated second electrical connector (e.g., wire) connecting the distal (e.g., second) electrode to the pulse generator. The proximal electrode may also be coaxially positioned at least over a portion of the spacer. In some examples the spacer extends proximally past the distal end of the proximal electrode further along at least a portion of the length of the proximal electrode (or a full length of the proximal electrode, or 1.25× the length of the proximal/first electrode, 1.5× the length of the first electrode, 1.75× the length of the first electrode, 2× the length of the first electrode, etc.). The spacers may generally be configured to prevent or reduce arcing and may be used (or formed) without the use of an adhesive/glue material, as in some cases the use of adhesive to bond and/or form the spacer may result in entrapped air bubbles that may result in arcing. Thus, in any of these apparatuses the spacer may be formed between the first and second electrodes without the use of an adhesive.

The apparatus for delivering a pulsed electric field described herein may comprise a handle and a tip region coupled to the handle (e.g., through an elongate shaft), the tip region may include a proximal electrode, a middle electrode, a tip electrode, a proximal spacer between the proximal electrode and the middle electrode, and a tip spacer between the middle electrode and the tip electrode. In some embodiments, the electrodes may be configured as bipolar or monopolar. In case of bipolar operation, a proximal electrode and a tip electrode may be a positive electrode, and a middle electrode may be a negative electrode, or the proximal electrode and the tip electrode may be the negative electrode, and the middle electrode may be the positive electrode. Both a proximal spacer and a tip spacer may be an insulative spacer or a conductive spacer, or one of the proximal spacer and the tip spacer may be the insulative spacer and the other of the proximal spacer and the tip spacer may be the conductive spacer.

According to additional aspects of the present disclosure, disclosed herein are apparatuses for delivering a pulsed electric field, the apparatus comprising an elongate shaft and a tip region extending distally from the elongate shaft, the tip region comprising a first electrode, second electrode, a spacer, and a support member. The second electrode of the tip region may be distal to the first electrode and may be configured to have a voltage differential relative to the first electrode. The spacer may be positioned between the first and second electrodes. The support member may be concentrically arranged relative to the first and second electrodes and may extend along at least a portion of a distance between the first and the second electrodes. Furthermore, the support member may be electrically separated in an axial direction from at least one of the first electrode and any components electrically coupled to the first electrode, or the second electrode and any components electrically coupled to the second electrode.

The first and second electrodes may be configured to have opposite polarities. In some examples, a spacing between the first and second electrodes may be adjusted. The distal (second) electrode may be configured as a needle with a penetrating distal tip, a trocar-type tip, a beveled tip, or any combination of the above, including a hybrid tip. Also, the apparatus may be configured to vary an exposure of a length of the distal electrode to a target tissue such that to vary a size of the treatment area. In various examples, the spacer may be insulative or conductive, and the apparatus may comprise more than one spacer. In some examples, the support member may overlap with at least one of the first and the second electrodes along a longitudinal axis of the tip region. Further, the overlap along the longitudinal axis may be approximately between 0.75 and 3 times a diameter of the support member, for example, approximately 1 time the diameter of the support member.

The apparatuses of the present disclosure may further comprise at least one insulative layer concentrically arranged along a portion of the tip region between the support member and the electrically separated first or second electrode. Such electrical separation of the support member from one of the first or second electrodes in the axial direction may be at least partially based on the voltage differential between the first and second electrodes and/or a dielectric characteristic (e.g., strength) of the insulative layer. The apparatus may be configured to deliver high voltage non-thermal electric pulses, for example, a sub-microsecond (e.g., nanosecond) electric pulses. The apparatuses of the present disclosure may be a part of a system comprising a pulse generator configured to deliver a high voltage pulsed electric field.

In some examples, the tip region of the apparatus may comprise an aspiration or infusion lumen configured to deliver a fluid to a target tissue and/or to provide negative pressure/suction to a target tissue. For example, the tip region may also comprise one or more ports or outlets, such ports may be located within a distal tip of the second distal electrode, or along a length of any or all of the first electrode, the second electrode, or the spacer. The apparatuses described above may be used in a method of treating a target tissue with a microsecond or sub-microsecond pulsed electric field.

Also described herein are apparatuses for delivering a pulsed electric field, the apparatuses comprising an elongate shaft and a tip region extending distally from the elongate shaft. The tip region may comprise a first electrode, a second electrode distal to the first electrode, a spacer between the first electrode and the second electrode; and a support member. The support member may be concentrically arranged relative to the first and second electrodes. The support member may also physically overlap with each of the proximal and distal electrodes along a longitudinal axis of the tip region and be electrically separated radially and axially from at least one of the first electrode and any components electrically coupled to the first electrode, or the second electrode and any components electrically coupled to the second electrode.

In particular examples, first and second electrodes of apparatus(es) herein may be configured to have a voltage differential relative to one another, and a support member of the apparatus may be electrically separated from the first electrode.

The apparatuses of the present disclosure may, in embodiments, further comprise one or more insulative layer(s) and/or adhering material(s) concentrically arranged along a portion of the tip region between the support member and the electrically separated electrode, e.g., first electrode. Such electrical separation of the support member from, e.g., the first electrode may be by a gap in the axial direction (e.g., a longitudinal spacing or distance) that is at least partially based on the voltage differential between the first and second electrodes and/or a dielectric strength of the insulative layer(s). The apparatus may be further configured to deliver high voltage electric pulses, for example, a sub-microsecond (e.g., less than 1000 nanoseconds) electric pulses.

As also provided above, in some examples, the tip region of the apparatus may also comprise an aspiration or infusion lumen configured to deliver a fluid to a target tissue and/or to provide negative pressure/suction to a target tissue. An apparatus may comprise one or more ports or outlets of the aspiration or infusion lumen. Such ports may be located within a distal tip of the second distal electrode, and/or along a length of any or all of the first electrode, the second electrode, or the spacer.

Still further described herein are multi-modal apparatuses for delivering a pulsed electric field, the apparatuses further configured for infusion, aspiration, or both. For example, multi-modal apparatuses herein may comprise an elongate shaft and a tip region extending distally from the elongate shaft. The tip region may comprise a first electrode, a second electrode distal to the first electrode, and a spacer between the first electrode and the second electrode. The tip region may further comprise at least one aspiration or infusion lumen extending through at least a portion of the tip region, wherein the apparatus is configured to deliver a bipolar pulsed electric field to a target tissue and to perform one or more of the following: (a) deliver a fluid from the tip region to the target tissue, (b) provide a negative pressure at the tip region to assure a proper contact with the target tissue and/or to facilitate collection of a sample from the target tissue, or (c) both (a) and (b).

According to certain embodiments, a fluid delivered by apparatus(es) herein can be a solution, a medication, a drug, or a therapeutic agent. In examples, at least one infusion lumen of the apparatus extends all the way to a distal tip of the distal electrode. In some implementations, at least one infusion lumen terminates at one or more ports proximal to the distal tip of the distal electrode. In some implementations, a tip region of apparatuses described herein further comprise a support member concentrically arranged relative to the first and second electrodes, wherein the support member extends along at least a portion of a distance between the first and the second electrodes. In embodiments, the support member is electrically separated at least in an axial direction from at least one of the first electrode and any components electrically coupled to the first electrode, or the second electrode and any components electrically coupled to the second electrode.

In some examples, the first electrode of the multi-modal apparatus is electrically coupled to provide a first polarity, and the second electrode is electrically coupled to provide a polarity opposite that of the first electrode. In some examples, the tip region may have any combination of the features described herein, including any of the spacers, electrode fillets (rounded edges) and any appropriate configuration of the distal electrode, proximal handle, as well as vacuum and/or infusion outlets and length adjustment capabilities. For example, as mentioned above, in some examples the second (distal) electrode may be configured to have a tissue-penetrating shape, for example, a beveled shape. Also, a circumference (e.g., outer circumference) of the spacer may be same as the circumference of each of the first electrode and the second electrode. In some examples, the circumference of the spacer may be greater than a circumference of the electrodes.

Also described herein are systems for providing electric treatment. For example, the systems may include: an apparatus according to any examples of the present disclosure and a pulse generator configured to generate a plurality of electrical pulses having amplitude of at least 0.1 kV and, for example, a duration in microsecond or sub-microsecond ranges, including less than 1000 nanoseconds.

As stated above, methods of using any of the apparatuses described herein are also included. For example, described herein are methods of treating a tissue with a sub-microsecond pulsed electric field comprising: inserting a tip region of a treatment tool into a target tissue, wherein the tip region comprises a first electrode that is proximal to a tissue-penetrating second electrode at a distal end of the tip region; adjusting a length adjuster on a proximal handle of the treatment tool to adjust a proximal-to-distal distance between the first electrode and the second electrode; and applying a plurality of electrical pulses having an amplitude of greater than 0.1 kV and a duration of less than 1000 nanoseconds, to treat the target tissue.

The proximal-to-distal distance between the first electrode and the second electrode may be adjusted based on a size of the target tissue. In any of these methods, adjusting the length adjuster may comprise rotating an adjuster knob of the length adjuster clockwise or counterclockwise. Any of these methods may include adjusting the proximal-to-distal distance between the first electrode and the second electrode between 1 mm and 7 mm.

As mentioned, any of these methods may include applying suction through a vacuum channel and a vacuum outlet of the treatment tool to secure the target tissue against the first electrode and the second electrode. Thus, any of these methods may include applying suction through a vacuum channel and a vacuum outlet to secure the target region of the tissue against the first electrode and the second electrode. Alternatively or additionally, the methods may include infusing a solution, e.g., a saline solution, through an infusion channel and an infusion outlet. Methods herein may include infusing a therapeutic agent to advance treatment by combining the electric pulse treatment and another therapeutic treatment.

The methods described herein may include: inserting, percutaneously or endoluminally, a tip region of a treatment tool into a target tissue, the tip region comprising a first electrode proximal to a second electrode; and applying a plurality of electrical pulses having a pulse duration of less than 1000 nanoseconds while reducing peak electric field for a given potential and/or increasing hoop stress on the target tissue to prevent or at least reduce arcing between the first electrode and the second electrode.

Any of these methods may improve contact with the tissue and/or reduce the likelihood of arcing by adjusting the position and/or dimensions of the spacer between the electrodes. For example, described herein are method that include: inserting a tip region of a treatment tool into a target tissue, wherein the tip region comprises a first electrode that is proximal to a tissue-penetrating second electrode at a distal end of the tip region; radially expanding a spacer between the first electrode and the second electrode so that a circumference (e.g., diameter) of the spacer is greater than a respective circumference (e.g., diameter) of both the first electrode and the second electrode to reduce arcing; and applying a plurality of electrical pulses having an amplitude of greater than 0.1 kV and a duration of less than 1000 nanoseconds, to treat the target tissue. The spacer may be in the first, narrow-diameter configuration when inserted into the tissue and may be expanded once positioned and before applying the energy. The spacer may be expanded by controlling a driver (e.g., plunger, pusher, puller, etc.) that may be on the handle of the device. Expanding the spacer may increase the contact with the tissue between the electrodes.

In general, the methods described herein may be methods of treating a target with a microsecond or sub-microsecond pulsed field. Any of these methods may be methods of positioning the apparatus and/or methods of avoiding arcing, while creating an improved contact with the treatment area.

The methods described herein may include methods of treating a thyroid module. For example, a method comprises inserting percutaneously a tip region of a treatment tool into a target thyroid nodule, the tip region comprising a first electrode and a second electrode; and applying through the first electrode and the second electrode a plurality of electrical pulses having a pulse duration of less than 1000 nanoseconds to thyroid nodule while reducing peak electric field for a given potential and/or increasing hoop stress on the thyroid nodule to prevent or at least reduce arcing between the first electrode and the second electrode. Thyroid nodule may be a benign thyroid nodule.

According to other implementations, methods described herein may include method(s) of treating a target tissue with microsecond or sub-microsecond pulsed electric field using a multi-modal apparatus. The method(s) may comprise delivering a treatment tip region of the multi-modal apparatus to a target tissue, wherein the multi-modal apparatus comprises a tip region having a first electrode, a second electrode distal to the first electrode, a spacer between the first and second electrodes, and at least one infusion lumen. The method(s) may further comprise applying a positive pressure to the treatment tip region of the multi-modal apparatus to deliver a fluid through the tip region to the target tissue; maintaining the tip region in position at the target tissue; and applying the microsecond or sub-microsecond pulsed electric field to the target tissue. In certain examples, a target tissue may be pancreatic tissue and the method may comprise treating pancreatic masses such as, e.g., benign or malignant pancreatic mass(es), pancreatic cyst(s), pancreatic neuroendocrine tumor(s)s (pNET(s)). In other implementations, the method may comprise treating other target tissue and organs, for example, other GI lesion(s) and tissue.

The method may include applying suction through an aspiration/vacuum lumen and a vacuum outlet of the multi-modal apparatus to secure the target tissue against the first electrode and the second electrode, or the target tissue. The method may further include adjusting the proximal-to-distal distance between the first electrode and the second electrode. Methods herein may include infusing a therapeutic agent to advance treatment by combining the electric pulse treatment and another therapeutic treatment.

The present disclosure also provides for methods of treating a pancreatic tumor with microsecond or sub-microsecond pulsed electric field(s) using a multi-modal apparatus of the present disclosure. The method may comprise delivering a treatment tip region of the multi-modal apparatus to a region of the pancreatic tumor of a subject, wherein the multi-modal apparatus comprises a tip region having a first electrode, a second electrode distal to the first electrode, a spacer between the first and second electrodes, and at least one infusion lumen. The method may further comprise applying a positive pressure to the treatment tip region of the multi-modal apparatus to deliver a material through the tip region to the pancreatic tumor; maintaining the tip region in position at the pancreatic tumor; and applying the microsecond or sub-microsecond pulsed electric field(s) to the pancreatic tumor. The pancreatic tumor may be benign or cancerous.

The present disclosure further provide for methods of treating a target tissue with a multi-modal treatment tool, the method comprising: positioning a treatment tip region of the multi-modal treatment tool relative to a target tissue of a subject, the treatment tip region comprising a treatment electrode and one or more infusion and/or aspiration channels; applying a positive pressure to the treatment tip region such that a material is infused through the treatment tip region at or within the target tissue; applying a negative pressure to the treatment tip region to maintain a contact between the treatment tip region and the target tissue and/or to collect a sample from the target tissue; and applying pulsed electric field(s) to the target tissue using the treatment electrode of the treatment tip region. The method may comprise any of the steps of applying positive pressure, applying negative pressure, or applying pulsed electric field(s) while maintaining the multi-modal treatment tool at the target tissue. For example, the application of the pulsed electric field(s) may be before, after, or during the application of the negative pressure. Further, in some implementations, the infusion may be applied before or after the application of the pulsed electric field. The pulsed electric field may be sub-microsecond (e.g., nanosecond) pulsed electric field(s). For example, collecting the sample may comprise performing a biopsy of the target tissue.

Any of the methods described herein may be adjusted for treatment of a variety of anatomical structure(s) and tissue(s), including without limitation, muscular tissue, circulatory tissue, respiratory tissue, digestive tissue, nervous system tissue, reproductive organs tissue, cardiac tissue, just to new a few, and the method(s) may include any combination of steps of ablation, infusion and/or aspiration. Also, the multi-modal treatment tools used in such method(s) may include any of the feature described in reference to various examples and embodiments of the present disclosure.

Any method(s) described herein may include any step(s) or feature(s) described in any other method(s) described in the present disclosure.

All of the methods, apparatuses, and individual attributes thereof described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates an example of a system for delivering a high voltage, fast pulsed electrical energy.

FIG. 2 shows a perspective view of an example of an apparatus, configured as a treatment tool as described herein.

FIG. 3A shows a cross-sectional view of an example of a treatment tool with a vacuum and/or infusion tubing. FIG. 3B is a perspective view of an example of an enlarged treatment tip region that may be used with the treatment tool of FIG. 3A.

FIG. 3C is a cross-section through an example of a treatment tool as described herein.

FIG. 3D is a cross-section through another example of a treatment tool as described herein in which the spacer extends proximally past the proximal electrode.

FIG. 3E illustrates an example of distal portion of a treatment tool as described herein configured to expose a vacuum outlet/inlet and/or infusion outlet/inlet.

FIG. 3F is a cross-section through an example of a distal portion of a treatment tool positioned within an introducer shaft.

FIG. 3G illustrates an example of a treatment tool partially withdrawn from an introducer, for example, at a first deployment position.

FIG. 3H illustrates an example of a treatment tool further withdrawn from an introducer at a second deployment position.

FIG. 4 shows a cross-sectional view of one example of a handle of a treatment tool with an adjustable treatment length.

FIGS. 5A-5B show examples of a treatment applicator having an adjustable treatment length. FIG. 5A shows an example of the length adjustment mechanism and FIG. 5B shows an example of a tip region with the adjustable spacing between the electrodes.

FIG. 5C illustrates an example of a treatment tool with a proximal electrode having been at least partially retracted.

FIG. 5D is a perspective view of the treatment tool in the configuration shown in the example of FIG. 5C.

FIG. 5E is a cross-sectional view of an example of the treatment tool in configuration of FIG. 5C.

FIGS. 6A-6E show examples of handles of treatment tools/applicators as described herein. FIG. 6A shows an example of a pencil-style handle. FIG. 6B shows an exploded view of the example of FIG. 6A. FIG. 6C illustrates a side cross-sectional view of another example of a handle of a treatment tool similar to that shown in FIGS. 6A-6B. FIG. 6D shows an exploded perspective view of the handle of FIG. 6C, and FIG. 6E is an enlarged view of a portion of the exploded view of FIG. 6D.

FIG. 6F illustrates an example of an apparatus according to the present disclosure having a handle comprising an aspiration valve control and additional features for positioning components of the apparatus.

FIG. 6G illustrates an example of aspiration valve control of FIG. 6F in multiple operating states: active (on) position (left) and inactive (off) position (right).

FIGS. 7A-7E show examples of electric field distributions corresponding to the treatment tools having different features as described herein. FIG. 7A shows an example of baseline electric fields without various features of the present disclosure. FIG. 7B shows an example of an electric field distribution of a treatment tool having fillets (rounded corners). FIG. 7C shows an example of an electric field distribution of a treatment tool having a conductive spacer between the electrodes. FIG. 7D1 illustrates an example of a treatment tool having fillets on the electrodes and an insulative spacer with an outer diameter (OD)/circumference larger than the OD (e.g., circumference) of the electrodes, and FIG. 7D2 illustrates the corresponding electric fields distribution for the treatment tool of FIG. 7D1. FIG. 7E illustrates electric field distributions corresponding to a treatment tool having a conductive spacer with the same OD as the electrodes and fillets on the electrodes. In FIGS. 7A-7C and 7D1-7E the scale shown on the right for the heat map extends from 1 to 10 and has been normalized to a relative scale based on the baseline (this scale does not reflect an actual voltage).

FIGS. 8A-8B show a front view and a side view, respectively, of an example of an apparatus having a trocar tip.

FIGS. 8C-8D show a front view and a side view, respectively, of an example of a treatment applicator having a conical tip as described herein.

FIGS. 8E-8F show a front view and a side view, respectively, of an example of a hybrid tip according to the present disclosure.

FIGS. 9A-9F show examples of tip regions of apparatuses as described herein. FIG. 9A illustrates an example of a tip region having a hybrid tip and a spacer with the same circumference as electrodes. FIG. 9B illustrates an example of a tip region having a trocar tip and a spacer with a circumference greater than the electrodes. FIG. 9C illustrates an example of a tip region having a trocar tip and a conductive spacer with the same circumference as electrodes.

FIG. 9D illustrates an example of a tip region having a conical tip and a conductive spacer with the same circumference as electrodes. FIG. 9E illustrates an example of a tip region configured as a trocar tip, having a spacer with a circumference that is greater than the OD of the electrodes, in which the electrodes include fillets. FIG. 9F shows an example of a fillet that may be used in various examples of the applicators of the present disclosure in combination with other features.

FIGS. 10A-10D show examples of tip regions with three electrodes as described herein. FIG. 10A illustrates an example of the tip region with three electrodes and FIG. 10B is a cross-sectional view of the tip region of FIG. 10A. FIG. 10C illustrates another example of a tip region with three electrodes. FIG. 10D is a cross-section view of the tip region of FIG. 10C.

FIG. 11 illustrates an example of adjustable length of exposure of a distal electrode in three exemplary positions of a treatment tip of the treatment device.

FIG. 12A illustrates an example of an apparatus comprising a treatment electrode configured as a piercing needle electrode having a lumen, and an optional stylet. FIG. 12B provides a cross-sectional view of the treatment device of FIG. 12A with no optional stylet present.

FIG. 13 illustrates an example of bipolar apparatus comprising a piercing needle electrode and a lumen (e.g., infusion lumen).

FIG. 14 illustrates an example of an apparatus comprising an electrode, aspiration lumen with an optional stylet.

FIG. 15 illustrates example of apparatus comprising a lumen for aspiration and treatment electrode(s) therein.

FIG. 16 illustrates a cross-sectional view of an example of a distal end of an apparatus (e.g., treatment applicator) comprising a support member, such as supportive bracing.

FIG. 17 illustrates a cross-sectional view of an example of a distal end of a treatment applicator comprising another example of a support member (e.g., supportive bracing).

FIG. 18 illustrates a cross-sectional view of an example of a distal end of a treatment applicator comprising yet another example of a support member.

### DETAILED DESCRIPTION

Described herein are apparatuses and methods for delivering electric treatment to various anatomical structures of a subject (human or animal). While these apparatuses may be especially useful when applying high voltage, pulsed electric fields (e.g., nanosecond pulsed electric fields), the apparatuses and methods described herein may also or alternatively be used with other energy modalities, such as RF energy, microsecond or picosecond pulses, etc. The apparatuses and methods described herein can be used to treat lesions, tumors, nodules and other growth, diseases and conditions, for example, in a target tissue, including various anatomical structures. Such target tissue may include tissue of various anatomical structures accessible, for example, via needle penetration through the skin and/or other percutaneous access applications, or via endoscopic delivery, catheter-based delivery, other minimally invasive technique, etc. In some examples, apparatuses may be used via laparoscopic delivery and in other examples, apparatuses of the present disclosure may be used in catheter-based or endoluminal delivery. In some examples, apparatuses, systems and methods of the present disclosure may apply nanosecond pulsed electrical fields to treat a target tissue, such as to treat lesions, tumors, nodules and other growths on or within various organs, including muscular organs (e.g., smooth muscle, cardiac and skeletal muscle), circulatory organs (e.g., heart, arteries, veins), respiratory organs (e.g., lungs), abdomen and digestive organs (e.g., stomach, duodenum, intestine, liver, pancreas), urinary organs, (e.g., kidney, ureter, bladder), immune system organs (e.g., lymph nodes, bone marrow, thymus), nervous system organs (e.g., brain, spinal cord, nerve), endocrine organs (e.g., pituitary gland, thyroid, adrenal glands), reproductive organs (e.g., penis, vagina, prostate, uterus, testicle), and/or skeletal organs (e.g., bones). In certain examples as stated above, apparatuses herein are configured for use in an endoluminal space. According to some embodiments, apparatuses of the present disclosure are flexible, single use, sterile endoscopic device(s). Apparatuses designed for endoluminal use may be catheter-based and relatively longer than those used for percutaneous applications, e.g., comprising relatively longer, relatively more flexible, or relatively longer and more flexible catheter shafts, e.g., being suitably configured to pass through an endoluminal scope (EUS) or other endoluminal device. According to some embodiments described herein are apparatuses and methods for delivering electrical treatment in an endoluminal space, such as, e.g., for treating structures such as but not limited to the pancreas, liver, lung, mediastinal lymph nodes, solid tumors, prostate, adrenal glands, gastrointestinal stromal tumors (GISTs), mural lesions, kidney, esophagus, gastric wall (e.g., submucosal lesions), and cysts. In some examples, apparatuses are designed for use within the upper gastrointestinal tract, among other endoluminal spaces. In some examples, apparatuses herein can be used with imaging guidance, robotic guidance, or both. Apparatuses herein can be delivered endoluminally via a scope, such as EUS.

According to some examples, apparatuses herein are configured to deliver electrical treatment to a target site and are further configured to also or alternatively deliver fluid or other material (e.g., dispense saline, medication, nutrients, an immune stimulus, a drug (e.g., chemotherapeutic agent), radiotherapy, targeted inhibitor, etc.); also or alternatively aspirate/biopsy material (e.g., a collection of cells or tissue); or also or alternatively both (selectively) deliver material and collect material.

Further described herein are apparatuses designed to facilitate access to very small anatomical spaces, such as, e.g., spaces associated with human and non-human anatomy. For example, such small anatomical spaces may be associated with the treatment of pancreatic masses, peripheral lung anatomy, nerve(s) (e.g., ganglia), or other anatomical spaces/locations sharing similar size constraints or access challenge(s). In some implementations, such small size apparatuses may be needed for use in small animal models, such as, e.g., a mouse. Accordingly, apparatuses and features thereof later disclosed in more detail herein may be sized and configured to be suitable for use in anatomical spaces that may typically be challenging for traditional technologies and existing devices.

One example of the use of the apparatuses and methods of the present disclosure is for treatment of thyroid nodules or thyroid lesions. Benign thyroid nodules are a form of non-malignant hyperplasia of the thyroid gland. They can present a cosmetic nuisance (an unsightly bulge in the neck), and in more severe cases can interfere with swallowing or breathing or can cause pain or pressure. In these cases, partial or complete surgical thyroidectomy may be performed, potentially resulting in hypothyroidism, hypoparathyroidism, nerve damage leading to voice impairment and visible scarring. Non-surgical options like radiofrequency (RF) ablation have been used but carry a risk of collateral damage to the recurrent laryngeal nerve, blood vessels, and other critical structures. Non-thermal electric treatment, such as nanosecond pulsed electric fields, may be used to disrupt cellular organelles, to induce apoptotic-like regulated cell death (RCD) without causing collateral damage to noncellular collagen-rich tissues, nerves, and vessels. Nanosecond pulsed electric fields treatment is non-thermal and in combination with various features of the present disclosure provides substantial advantages to existing surgical excision or thermal ablation technology(ies), such as RF ablation. For example, in addition to reducing the risk of collateral damage as stated above, the use of nanosecond pulsed electric fields may also eliminate or substantially reduce scarring or fibrosis and may cause minimal post-procedural pain. Disclosure directed to the treatment of benign nodules should be interpreted as exemplary, such that any such disclosure should be interpreted as being applicable, uncontradicted, to the treatment of other target site(s) such as, e.g., cancerous tumors, lesions, and the like. Apparatuses and features thereof disclosed herein, uncontradicted, are suitable for use in any application, and for use in treating any disease or condition, for which the technology(ies) provided by the apparatuses are relevant.

While various following examples are described in reference to treatment of thyroid or a treatment of pancreatic cancer, it shall be understood that such references are just non-limiting examples for convenience of description and the devices and methods of the present disclosure apply and are intended for use in any target tissue and anatomical structures, as may be appropriate, for example, for percutaneous access, endoluminal access, or otherwise.

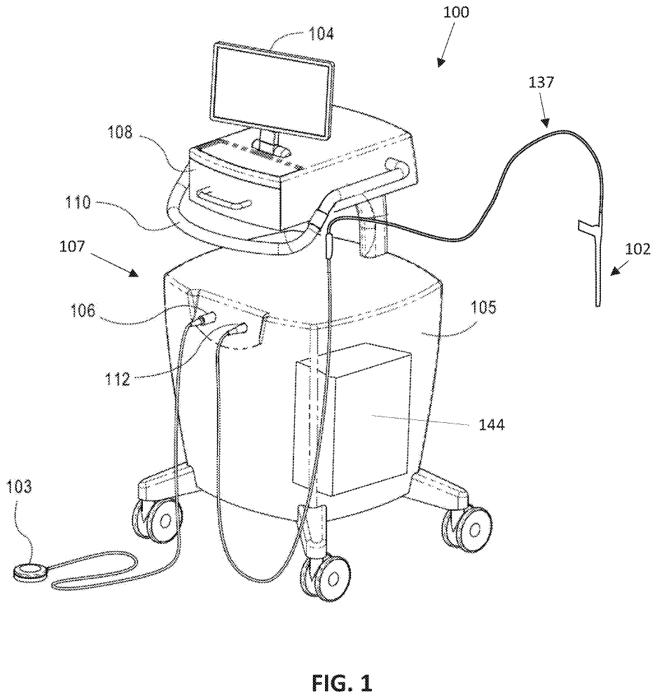

FIG. 1 illustrates one example of a system 100 for delivering high voltage, fast pulses of electrical energy that may include a treatment applicator/tool or apparatus 102 for delivering a pulsed electric field (e.g., sub-microsecond electric field), a pulse generator 107, footswitch 103, and user interface 104. Footswitch 103 is connected to housing 105 (which may enclose the electronic components) through a cable and connector 106. The apparatus 102 may include electrodes and is connected to housing 105 and the electronic components therein through a cable 137 and high voltage connector 112. The system 100 may also include a storage drawer 108 and a console handle 110. The system 100 may also include a holder (e.g., holster, carrier, etc.) (not shown) which may be configured to hold the tool 102. Examples of appropriate applicator tools 102 are described in greater detail below.

In some cases, the applicator tool 102 may include imaging, such as one or more cameras and/or fiber optics, for example, at or near the tip region of the tool. The camera(s) may be forward-facing and/or side facing. System 100 may be configured to display images (in real time, and/or recorded) in order to identify the target region(s).

A human operator may select a number of pulses, amplitude, pulse duration, and frequency information, for example by inputting such parameters into a numeric keypad or a touch screen of interface 104. In some embodiments, the pulse width can be varied. System 100 may include a controller 144 (shown schematically in FIG. 1), which may send signals to pulse control elements within system 100 or otherwise control operation of the pulse generator. The controller 144 may include one or more processors and may be coupled to the pulse generator either directly or indirectly. The controller may receive input from the one or more inputs and may provide output to the one or more outputs (e.g., monitors/touchscreens/interface 104, etc.). The controller may be a microcontroller. The controller may include control circuitry and may include or be coupled with a memory, communications (e.g., wireless and/or wired) circuitry, etc. The controller may be configured to coordinate the application of energy to the patient. In some embodiments, fiber optic cables are used which allow control signaling while also electrically isolating the contents of the metal cabinet with pulse generation system, e.g., the high voltage circuit, from the outside. System 100 may be battery powered instead of being powered from a wall outlet.

Tool 102 (also referred herein as an apparatus, treatment tool, or treatment applicator) may be hand-held (e.g., by a user) or it may be affixed to a movable arm of a robotic apparatus, and its operation may be at least partially automated or fully automated, including computer controlled.

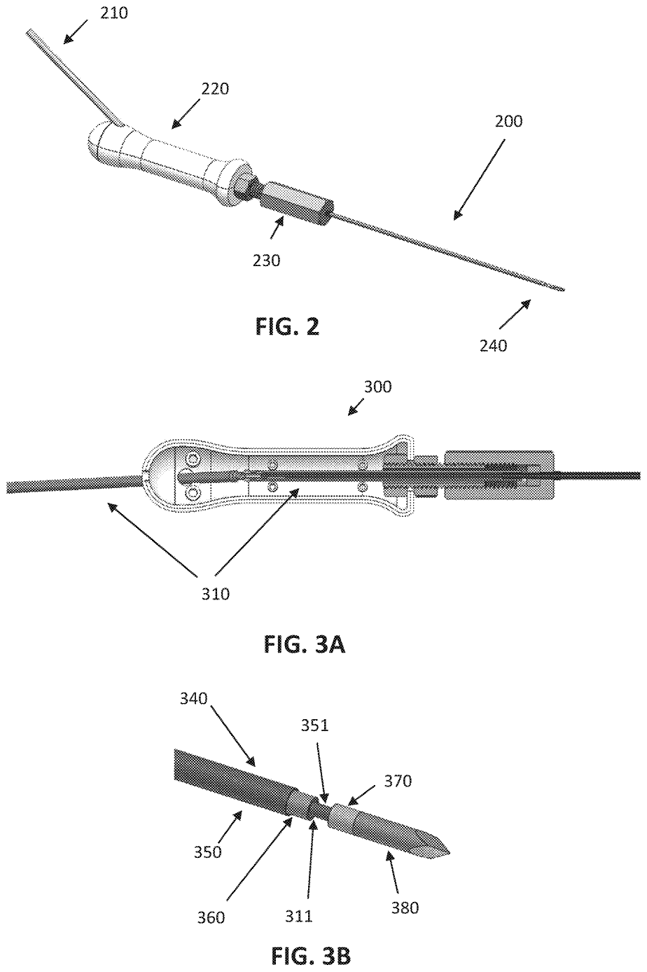

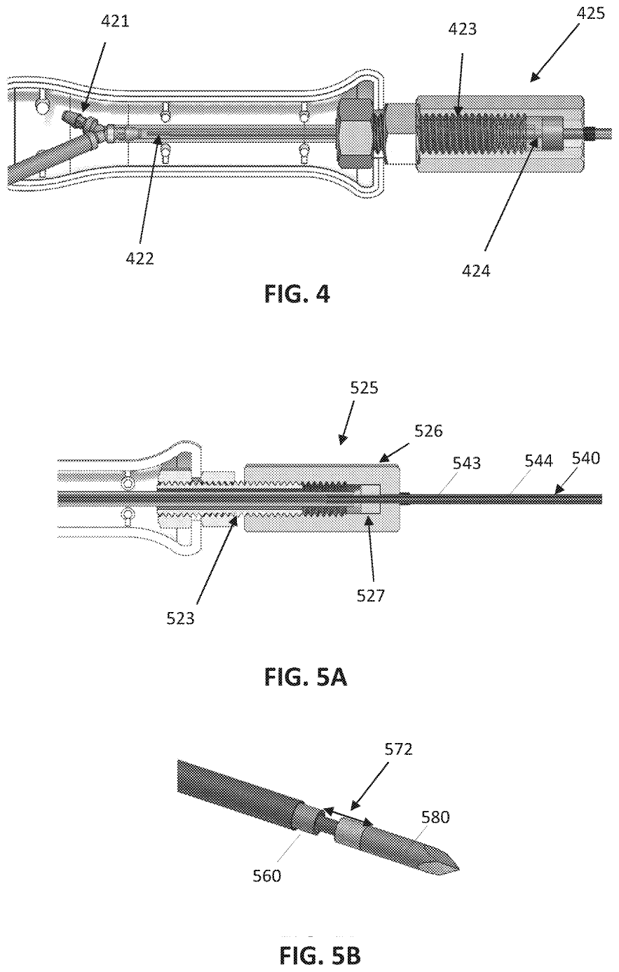

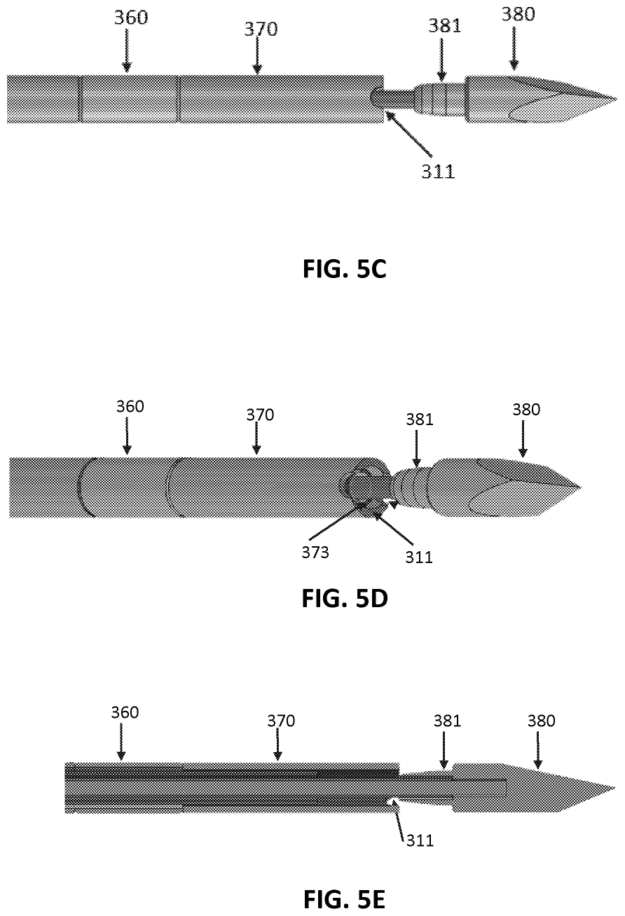

FIG. 2 illustrates the perspective view of an example of a treatment tool/apparatus 200 (which may be used with the system 100 of FIG. 1) as described herein. In this example, the tool 200 includes a vacuum channel and/or an infusion channel 210 (e.g., vacuum line), a handle 220, a tip region 240 and a length adjustment 230 (length adjuster) operably coupled to the handle 220 and to the tip region 240. The length adjuster 230 is configured to adjust a distance between a first electrode and a second electrode as described in more detail in reference to FIGS. 4 and 5A-5C. In the example of FIG. 2, the treatment tool 200 also incorporates a vacuum assist feature including the vacuum channel 210, which allows the apparatus to evacuate extra air from around the electrodes, which in turn reduces the likelihood of arcing between the electrodes. The vacuum channel 210 may be connected to the source of vacuum or suction, for example, a pump or a self-contained vacuum source within the tool 200 itself, for example, within the handle 220. The treatment tool 200 may be connected to a pulse generator (e.g., system 100 of FIG. 1) via electrical cable (not shown). The treatment tool 200 may be connected to the pulse generator via electrical cable. The electrical cable may be electrically isolated (and insulated) from the tool 200, and therefore the operator's hand(s) in the hand-held implementations, by one or more isolation elements. In some examples the handle 220 may be a plastic or insulated housing for the user to hold and apply the pulses to the load. It should be understood, however, that the term “handle”, as used herein, is intended to describe a proximal portion of the treatment applicator and is not limiting. It refers to any structure to support, hold, or attach to the treatment tip region with the electrode portion of the device, whether it is intended to be hand-held, or attached to a robotic arm, or for percutaneous or other minimally invasive applications. In some examples the handle may be configured to be hand-held and may include a manual grip. In some examples, the handle may be configured to be held by a robotic manipulator (e.g., arm, etc.).

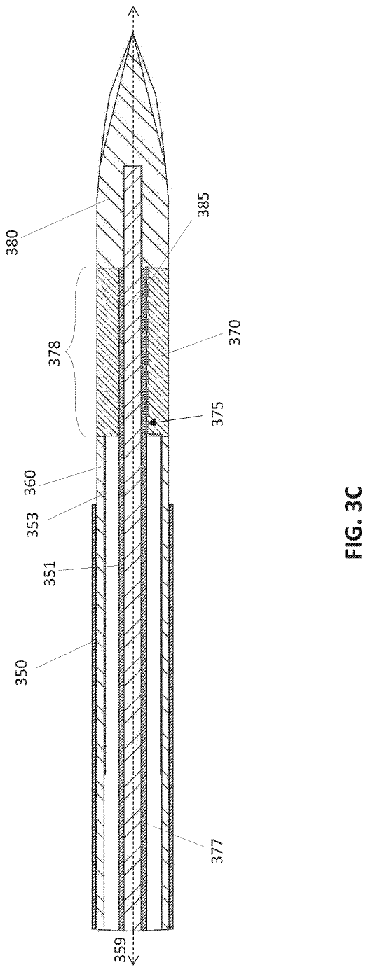

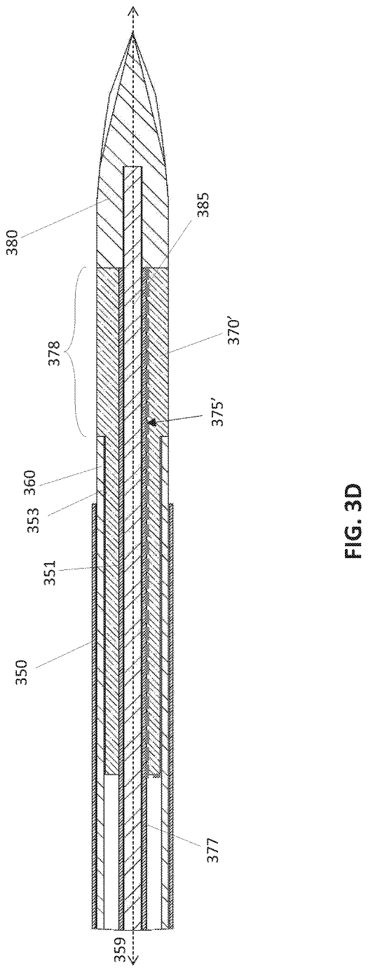

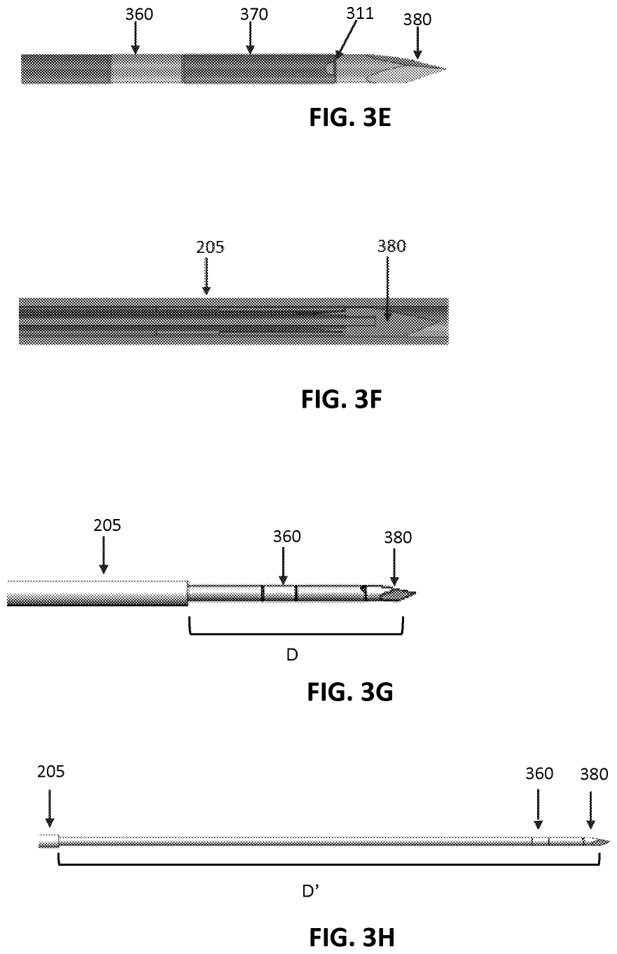

FIGS. 3A-3B illustrate in more detail some elements, including a vacuum-assist feature and an infusion output, that may be incorporated into any of the apparatuses described herein. FIG. 3A shows an example of a section through a treatment tool 300 having vacuum channel 310, which may alternatively or additionally be an infusion channel 310. The vacuum and/or infusion channel 310 may be disposed at least partially inside the handle. The application of vacuum and/or infusion may be controlled via an external vacuum pump or infusion pump (e.g., a pump capable of providing positive pressure) (not shown in the drawing). Such pump(s) may be mechanical (e.g., comprising a motor) or manually operated (such as, e.g., a manually operated syringe). The vacuum channel and vacuum outlet may be used for suction/vacuum to remove any potential air gap between the electrodes. The vacuum channel and vacuum outlet may be used to collect material from a treatment site, such as, e.g., a sample of cells or tissue. Such feature facilitates the ability to collect material from a target site, with a treatment tool tip region comprising one or more treatment electrodes in place in the target site, without a need to remove and then replace the treatment electrode(s) from the target site. In some examples, the same channel (e.g., tubing) 310 may be used to deliver a saline solution (or multiple different types, e.g., concentrations, of saline solution), which may be used with a vacuum or by itself. In some examples, sterile saline may be infused and could fill the gaps with a conductive solution providing an extra electrical pathway which could potentially enlarge the actual treatment size. In some examples, a therapeutic agent (e.g., a medication) may be infused. In any of these apparatuses, both a vacuum/suction and an infusion channel may be included, so that suction and infusion may be applied concurrently.

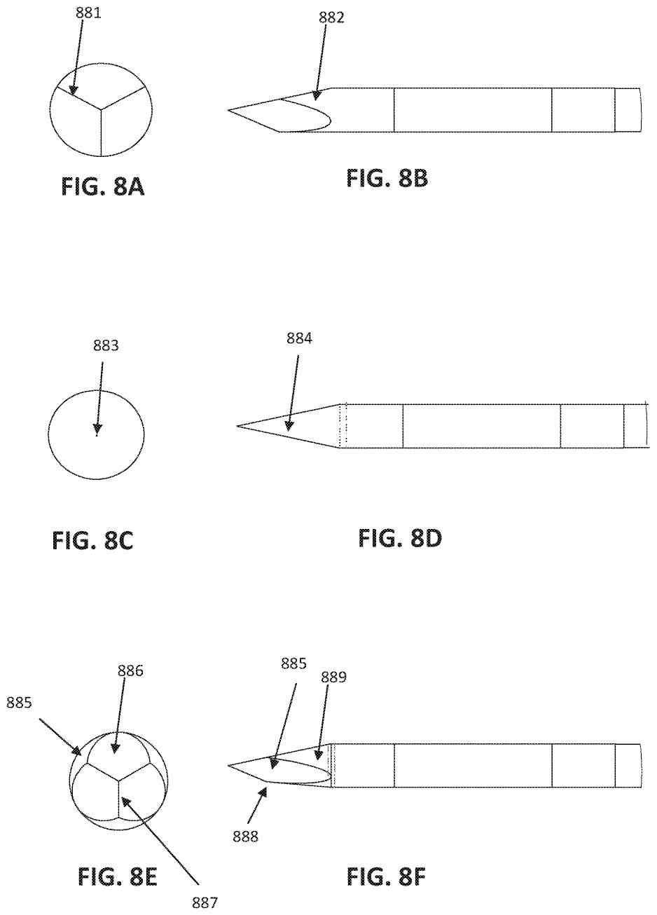

FIG. 3B shows a perspective view of the tip region 340 of the treatment tool 300, which comprises a first (e.g. proximal) electrode 360, a second (e.g., distal or tip) electrode 380, an exterior insulation 350 and an inner insulation 351. While in this example the treatment tool is configured for bipolar application, it should be understood that it may be used also for monopolar energy application. The first electrode and the second electrode may comprise a conductive material, such as a conductive metal (e.g., a stainless-steel material). Since, in certain examples, an apparatus of the present disclosure is configured for percutaneous insertion, the tip region 340 may be configured to penetrate the skin and be advanced into the target area for treatment. In certain examples, such a tip may be present for purposes of penetrating or piercing target anatomy, such as, e.g., an organ. For example, the second electrode 380, which is at a distal end or forms a distal end of the tip region 340, may be a tissue-penetrating electrode in the form of a bevel, a cone, a trocar (such as 3-sided trocar), or a hybrid tip, depending on requirements. The second electrode (in this example, the distal electrode) may be longer than the first electrode (in this example, a proximal electrode). Other examples of electrodes at the distal end of the tip region (which may be also referred to as the distal electrode) are described in more detail in reference to FIGS. 8A-8F, below. Additional or alternative examples of apparatus features (e.g., tip region design element(s)) configured for or compatible with use in, e.g., endoluminal applications, are described elsewhere herein.

The exterior insulation 350 and the inner insulation 351 may include, for example, a polymeric insulator, such as a polyimide material. The vacuum and/or infusion channel 310 shown in FIG. 3A may connect to a vacuum outlet/inlet and/or infusion outlet/inlet 311 as shown in FIG. 3B. In FIG. 3B, the outlet 311 may be disposed between the first (proximal) electrode 360 and an inner insulation 351. Each electrode may be connected to a high voltage wire (not shown) within the handle, which may connect back to the pulse generator, such as the one shown in FIG. 1. For example, a first wire may connect to the first electrode 360 and a second wire may connect to the second (distal) electrode 380. The exterior insulation 350 may insulate the first wire and the inner insulation 351 may insulate the second wire. The electrical lines connecting to the first and/or second electrode may be wires, cylinders, cables, meshes, etc. For example, the electrical line connecting the first electrode 360 to the pulse generator may be coaxially arranged relative to the electrical line connecting the second electrode 380 to the pulse generator. Each pole (e.g., the first electrode and the second electrode) may be electrically isolated. The isolation may be improved by separating and at least partially insulating the poles with a spacer 370 (e.g., a polyimide insulative spacer) that may be disposed between the first electrode 360 and the second electrode 380 and may be configured to electrically isolate the first electrode 360 and the second electrode 380 from each other. The target region may be treated by electrical energy flowing in a bipolar manner between the first electrode 360 and the second electrode 380.

As mentioned, any of the apparatuses described herein may be configured to prevent or reduce arcing, including in particular, arcing between the first (e.g., proximal) and second (e.g., distal) electrodes. In any of the apparatuses described herein the first electrode and the second electrode may be separated from each other by a relatively long minimum clearance (e.g., creepage path) distance. Although the spacers described herein may be generally configured to prevent or reduce arcing, the use of adhesive (e.g., glue) to bond and/or form the spacer may result in entrapment of air bubbles that may in turn lead to arcing. Thus, in any of these apparatuses the spacer may be formed between the first and second electrodes without the use of an adhesive. Spacers described herein may comprise features facilitating infusion, aspiration, or both, such as, e.g., infusion port(s) as described in conjunction with the exemplary treatment tool shown in FIG. 3E. Spacers 370 can comprise any suitable length, such as, for example, having a length of between about 1 mm and about 10 mm, between about 1 mm and about 8 mm, about 1 mm and about 6 mm, or between about 2 mm and about 5 mm, and any combination in between.

Further, in any of these apparatuses the spacer between the first electrode and the second electrode may be configured to maximize or increase the standoff distance (e.g., the minimum clearance or electrical creepage distance). For example, FIG. 3C shows an example of a device including a spacer 370 that is positioned between the proximal electrode 360 and the distal electrode 380. The proximal end of the spacer in this example extends only to the distal end of the proximal electrode. FIG. 3D shows another example in which the spacer 370′ extends further proximally, and in this example extends proximally to the proximal electrode 360. Spacers that extend proximally to the proximal electrode may be particularly advantageous as they may increase the minimum clearance or electrical creepage distance 375, 375′ between the electrodes. In FIG. 3C the minimum clearance or electrical creepage distance 375 extends along the spacer and is approximately the distance between the electrodes 378 (as measured from the outside of the device). In contrast, the example shown in FIG. 3D illustrates a spacer 370′ that extends proximally past the proximal electrode, so that the minimum clearance or electrical creepage distance 375′ is more than twice the minimum clearance or electrical creepage distance in the example shown in FIG. 3C.

Thus, in any of these apparatuses the first electrode may be separated from the second electrode by a spacer that extends between the first electrode and the second electrode, and may extend past one or both of these electrodes, so that a minimum electrical clearance (electrical creepage) distance between the electrodes is greater than the distance separating the first and second electrodes (e.g., 1.5× or greater, 1.75× or greater, 2× or greater, 2.25× or greater, 2.5× or greater, 2.75× or greater, 3× or greater, 3.25× or greater, 3.5× or greater, etc.), for example, when measured from the outside of the apparatus. In some examples, as shown in FIG. 3D, the spacer 370′ may extend proximally to the proximal electrode 360, to the proximal end of the distal electrode 380, which may increase the standoff distance without having to increase the distance between the electrodes.

In both FIGS. 3C and 3D the apparatus includes a proximal electrode 360. The proximal electrode may be formed as a ring extending partially or completely around the outer surface of the device. The proximal electrode may have an insulated inner surface 353. The proximal electrode 360 may be formed of a cylindrical, electrically conductive material that is arranged coaxially over the internal wire 385 or other electrical connection to the distal electrode 380 and over the spacer 370. A portion of the cylindrical, electrically conductive material that does not form the electrode (e.g., the region proximal to the proximal electrode) may be insulated 350, as shown in FIGS. 3C and 3D. The distal end of the apparatus is configured as a tissue-penetrating distal electrode 380 that is electrically coupled to the pulse generator via the internal wire 385 or other electrical connection. In the sectional view shown in FIGS. 3C and 3D the apparatus (or at least the distal end of the apparatus shown) is radially symmetric about the long axis 359 of the apparatus. In this example, the insulated outer surface or outer insulator 350 may be a thin layer adjacent to the proximal electrode (e.g., at the top and the bottom). As mentioned above, the inner surface 353 of the first (proximal) electrode 360 (e.g., the radially inward surface) is also insulated. In addition, the electrical conductor (e.g., wire) connecting the distal electrode 380 to the pulse generator may be insulated by an inner insulator 351, e.g., between the spacer 370, 370′ and the central wire 385 leading to the distal electrode 380. In the example device of FIG. 3D, the length of the minimum clearance distance/minimum creepage distance 375′ is greater than 2.75 times (e.g., greater than about 3×) the external distance 378 (actual distance between the distal end of the proximal electrode 360 and the proximal end of the distal electrode 380). In any of the apparatuses described herein the more proximal end region 377 between the outer (e.g., electrode 360) portion and the inner (e.g., wire connector 385) portion may be open; the corresponding distal end region is taken up by the spacer. In some examples this region 377 may be filled with material, including in some examples a compressible material.

Furthermore, in the example apparatus shown in FIG. 3D, the spacer 370′ is held within the apparatus in a fixed position without the need to use a glue bond or any other adhesive, since the length of the spacer underlying and extending proximally of the proximal electrode may be sufficiently long enough to avoid arcing without the need for a glue bond as an insulator. The spacer may be either insulative or conductive in different embodiments.

As mentioned, the spacer between the electrodes may be insulating or in some examples it may be a conductive spacer. In any of these examples the spacer may be a balloon that may be filled with an insulating or a conductive material. In some examples the spacer may be filled with air.

As mentioned above, any of the apparatuses/treatment tools described herein may be configured to include suction (e.g., vacuum) to assist in holding the electrodes to the tissue to be treated as well as reducing arcing. The vacuum (suction) may pull the tissue onto the electrodes (e.g., the first electrode) and/or may maintain contact with the electrodes. The use of a vacuum may remove or reduce air gaps between the electrodes and the tissue, which may reduce arcing and otherwise improve contact with the tissue. In some variations suction may be automatically or manually applied before activating the application of a high voltage, fast pulsed electrical energy. Suction may be applied to a predetermined level to prevent damage to the tissue. Once the energy has been applied, the suction may be released, automatically or manually. In addition to holding the tissue to be treated, and/or reducing arcing, suction may be used, for example, to collect tissue for external analysis. However, it should be understood that the examples and implementations described herein may be used without the vacuum.

FIG. 3E demonstrates an example of a distal region of a treatment tool comprising a proximal electrode 360, a distal electrode 380, and an electrode spacer 370 positioned between the proximal and distal electrodes. FIG. 3E illustrates an infusion or aspiration outlet (port) 311 located, in this example, at the distal end of spacer 370. Infusion or aspiration outlet 311 may facilitate or otherwise participate in the aspiration (thus, e.g., serving as an aspiration/vacuum or collection outlet), dispensation/infusion (thus, e.g., serving as a dispensation outlet), or both. In various implementations, any number of outlets may be present, such as 1, 2, 3, 4, or, e.g., 5 or more. Such outlets can take any shape and be of any suitable size. While outlet (port) 311 is shown in FIG. 3E at a distal end of the spacer 370, in other implementations such one or more outlets or ports may be positioned at a proximal or distal end of spacer 370. In various implementations, outlet(s) 311 may be present within and along a wall of an electrode spacer 370. In examples wherein two electrodes, such as a proximal electrode 360 and a distal electrode 380, are repositionable relative to one another, as, e.g., illustrated in FIGS. 5C-5E, outlets 311 may facilitate infusion, aspiration, or both regardless of the relative positioning of the electrodes. In some configurations (not shown) outlets 311 may be within one or more electrodes (for example a proximal electrode, distal electrode, or both), for example, at a proximal or distal end of one or more electrodes or within a wall along a length of the respective electrode(s). Regardless of configuration, outlet 311 is capable of one or both of infusion and aspiration regardless of the relative positioning of one or more electrodes.

FIG. 3E does not show an internal infusion/aspiration chamber or lumen through which saline or other drug/medication may be injected through and out around the one or more outlets 311 of FIG. 3E, however such chamber/lumen is shown in the following additional figures. In examples illustrated by additional figures herein, the proximal portion of the treatment region of the apparatus (e.g., an assembly of the spacer and proximal electrode) may be retracted to establish a longer distance between the proximal electrode and the distal electrode. An extended distance between electrodes may increase the size of the treatment area. Retracting the proximal portion to establish a longer distance between the proximal and distal electrodes may expose a vacuum outlet/inlet and/or infusion outlet/inlet 311 associated with one or more internal lumens (not shown in FIG. 3E). As previously stated, the exposed lumen(s) may, for example, be used to dispense or otherwise deliver a material such as a fluid, e.g., saline or a therapeutic agent (e.g., medication/drug). The exposed lumen(s) may also or alternatively be used to collect material sample(s), such as sample(s) of cells or tissue from a target site. The distal electrode 380 embodied in the example of FIG. 3E comprises a trocar or beveled shape tip design, facilitating tissue access. The retraction of the proximal electrode/spacer assembly and the resulting increase in distance between the proximal and distal electrodes, accompanied by the exposure of the vacuum outlet/inlet and/or infusion outlet/inlet 311′, is illustrated in the examples of FIGS. 5C-5E.

In various examples, a treatment tool such as that exemplified in FIG. 3E is capable of aspiration or delivery of material through one or more outlets or ports when the proximal electrode is not retracted, e.g., when the proximal electrode is positioned as close to the distal electrode as allowed by the device. In other examples, a treatment tool is configured to provide for aspiration and/or infusion by retraction of the proximal electrode/spacer assembly from the distal electrode at least partially, exposing at least a portion of a vacuum outlet and or infusion outlet.

During delivery to the target tissue, a treatment tool, such as one shown in FIG. 3E, may reside at least partially or entirely within an introducer 205. FIG. 3F is a cross-section view of such a configuration, with introducer 205 presented as encompassing the entire length of the treatment tool, whereby the distal electrode 380 is positioned within a shaft of introducer 205.

The outer diameters of each of the proximal electrode, distal electrode, and electrode spacer are configured such that they may be passed through one or more other components of an apparatus/system in or with which the treatment tool is being used, such as, e.g., within an introducer shaft. In some implementations, treatment tools of the present disclosure may be specifically designed to be used in applications where the target or subject is very small, as, for example, in locations associated with pancreatic masses, peripheral lung anatomy, nerve(s) (e.g., ganglia), and the like, or anatomical spaces/locations sharing similar size(s), and/or access challenge(s). Therefore, in some embodiments, apparatuses herein can be designed to have an outer diameter no larger than about 3 mm, no larger than about 2.9 mm, 2.8 mm, 2.7 mm, 2.6 mm, 2.5 mm, 2.4 mm, 2.3 mm, 2.2 mm, 2.1 mm, 2.0 mm, or even smaller, such as 1.9 mm, 1.8 mm, 1.7 mm, 1.6 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, or, e.g., no larger than 0.4 mm. The proximal electrode, distal electrode, and electrode spacer in some examples may comprise at least substantially the same or the same outer diameter or circumference. In other examples, a proximal electrode may have a detectably or significantly larger, or alternatively a detectably or significantly smaller, outer diameter/circumference than one or both of the distal electrode and the electrode spacer. In some examples, an electrode spacer may have a detectably or significantly larger, or alternatively a detectably or significantly smaller, outer diameter/circumference than one or both of the proximal electrode and the distal electrode. In further examples, a distal electrode may have a detectably or significantly larger, or alternatively a detectably or significantly smaller, outer diameter/circumference than one or both of the proximal electrode and the electrode spacer. Further, in certain examples, one or more of the proximal electrode, distal electrode, and electrode spacer may have outer circumference which vary across their respective lengths. In some examples, one or more of the proximal electrode, distal electrode, and electrode spacer may have modifiable outer diameters, e.g., diameters which change over the course of their application or use in procedure(s).

FIGS. 3G and 3H illustrate examples of deployment of the tip region of the treatment tool from the introducer 205. FIG. 3G illustrates the treatment tool comprising proximal electrode 360 and distal electrode 380 deployed from introducer 205 at a distance (D). Distance (D) can be any suitable distance, such as, for example, between 0.1 cm and 10 cm, between 1 cm and 8 cm, between 1 cm and 4 cm, etc. The deployment of the tip region comprising distal and proximal electrodes can be controlled to set at a desired distance, depending on a particular target tissue and/or the needs of the procedure. FIG. 3G demonstrates a shorter deployment distance D, for example, at 2 cm, while FIG. 3H demonstrates a longer deployment distance D′, for example, between 6 cm and 15 cm, for example, 8 cm. FIGS. 3G and 3H illustrate the versatility of apparatuses herein, having the ability to adapt to apply electrical treatment to a wide range of target sites. Further, as has been described, the treatment tools of the present disclosure may provide for the length adjustment between the proximal and distal electrodes.

FIGS. 4 and 5A-5C demonstrate an example of a length adjustment feature (length adjuster) that may be present on any of the treatment tools described herein. FIG. 4 shows an example of a handle and length adjuster 425 of the treatment applicator according to the present disclosure. As described in reference to FIGS. 3A-3B, the first and the second electrodes may be each connected to a respective high-voltage (HV) wire. The HV wire allows delivery of the high-voltage electrical field and may be formed of any appropriate material. In the example apparatus shown in FIG. 4, the first wire 422 is connected to the first electrode (not shown) and disposed within the handle and the length adjuster 425. The first wire may be insulated with the exterior insulation (as stated in reference to FIG. 3B) and routed, for example, through a threaded rod 423. A second wire 424 may be connected to the second electrode and may be insulated with the inner insulation (not shown) as stated in reference to FIG. 3B. The second wire 424 may be introduced through a luer-type standard fitting 421 and it may be sealed off for the vacuum or the infusion. In some examples the second wire 424 may be slidably disposed within the device, including within the elongate shaft 540 (shown in FIG. 5A), so that as the spacing between the electrodes is increased or decreased (e.g., by actuating the length adjuster 425).

In some examples, the wire (electrical connector) includes a region formed of a hypotube (e.g., stainless-steel or other conductive material) which may be full hard, welded and drawn to tight tolerances and which is an effective component for applications requiring strength, uniformity, and corrosion resistance. It can be a high-performance alloy for use in non-implantable medical devices. For example, the wire may include a minimum of 18% chromium and 8% nickel with a maximum of 0.08% carbon. It can be in the chromium-nickel austenitic alloy family.

In the examples shown in FIGS. 2, 3A-3B, 4 and 5A-5B, the length adjuster(s) shown are configured as a threaded member coupled to an extendable (e.g., telescoping) member that may rotate to longitudinally move a first elongate member, to which either the first or second electrode is attached, relative to a second elongate member, to which the other electrode (e.g., the second or first electrode) is attached. The first elongate member may be concentrically arranged relative to the second elongate member. Rotational movement of the length adjuster results in longitudinal movement of the first elongate member relative to the second elongate member, and therefore a change in the spacing between the first electrode and the second electrode at the distal ends of the first elongate member and the second elongate member. The length adjuster may be calibrated (and labeled) so that a specified rotational movement may result in a specified longitudinal movement and, therefore, a specified increase or decrease in spacing (depending on the direction of rotation). In some examples the length adjuster may be configured so that rotation of the length adjuster does not rotate the first or second elongate member. The first and second elongate members may be held within or may form a part of the elongate shaft 540 extending from the handle in a proximal-to-distal direction. The tip region (including the first and second, or more, electrodes) may extend distally from the tip region. The distal tip (and the electrodes thereon) may be formed at the distal end of the shaft or may be coupled to the elongate shaft.

FIG. 5A shows one example of adjusting a treatment length, i.e., adjusting a distance between the first electrode and the second electrode. The length adjuster 525 in this example includes a threaded rod 523 and an adjuster knob 526 disposed on the threaded rod 523, which may allow for the apparatus to adjust spacing between the electrodes, for example, to treat different treatment lengths. As the adjuster knob 526 is rotated clockwise and/or counterclockwise, it pushes against a distance adjustment stator 527, which is attached to the first electrode through an elongate member 543, thereby advancing or retracting the (inner) elongate member 543, and moving the first electrode in or out, which in turn increases or decreases the spacing between each electrode, as illustrated in FIG. 5B. In this example, the second, tissue-penetrating distal electrode 580 is rigidly coupled to the inner elongate member 543, while the first, more proximal, electrode 560 is rigidly coupled to the outer elongate member 544. The example shown in FIG. 5A is non-limiting example, and other length adjustment mechanisms may be used.

For example, a length adjustment mechanism may be configured to longitudinally slide the inner elongate member relative to the outer elongate member by driving a slider on the handle proximally or distally. In some examples the handle may include a gear or gearing for controlling the relative longitudinal movement of the inner and outer elongate members. In some examples a hydraulic or pneumatic mechanism may be used to drive the separation or contraction of the first and second electrodes, e.g., by controlling longitudinal movement of the inner and outer elongate members to which the first and second electrodes are coupled.

In use, the operator may adjust or direct adjustment of the distance between the first electrode and the second electrode depending on the size of the target region so that the electric field can pass through the target tissue properly and efficiently. As mentioned, FIG. 5B shows an example of the tip region of an apparatus with the adjustable treatment length between the electrodes. The spacing between the first 560 and second 580 electrodes may be adjusted as shown by arrow 572. The spacing between the electrodes may be, for example, from between about 1 mm to 10 mm, 1 mm to 9 mm, 1 mm to 8 mm, 1 mm to 7 mm, 1 mm to 6 mm, 2 mm to 8 mm, 2 mm to 7 mm, 2 mm to 5 mm, etc., depending on the application and the energy required. This distance may be adjustable according to the size of the target region. In some examples the region between the electrodes may include a spacer, as mentioned above. The spacer may be configured to expand and contract as the spacing between the electrodes increases or decreases. For example, the spacer may be configured to stretch or compress.

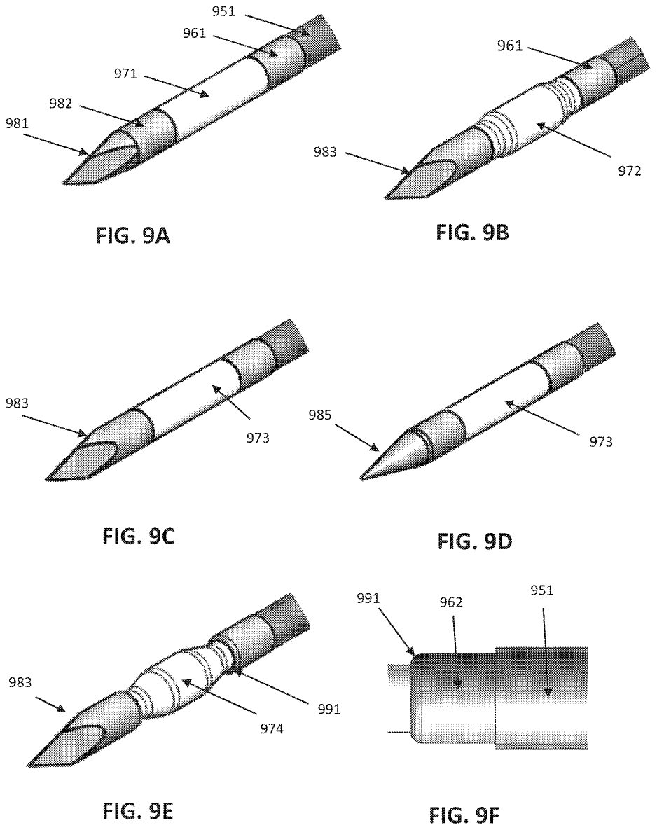

For example, in any of the apparatuses described herein the spacer may be configured to radially expand and/or contract from a flush configuration (see, e.g., the spacer of FIG. 9A, described below) to a more radially expanded configuration (see, e.g., FIGS. 9B and E). Alternatively in some examples the spacer may be configured to retract to have a radial diameter that is equal or less than the outer diameter of the rest of the apparatus (e.g., the proximal electrode). Radial expansion of the spacer may allow the device to create a more intimate contact with the tissue, for example, after insertion and during application of the electric energy. For example, the apparatus may be adjusted to decrease the circumference (e.g., diameter) of the spacer, e.g., by pushing the inner member, for example, by pushing an electrical line or wire coupled to the distal electrode, or to a support coupled thereto, distally. In this example, releasing the force previously applied to push the inner member distally may permit the spacer to revert to expand radially outwards, and/or force may be applied (e.g., by pulling the inner member or wire proximally) to drive the spacer outwards to expand its circumference. Alternatively, in some configurations the apparatus may be configured so that in the neutral configuration the spacer has a circumference/diameter that is relatively small, or smaller than the circumference/diameter of the spacer when force is applied, e.g., pulling the inner member (distal electrode) proximally relative to the outer member (e.g., proximal electrode). Thus, the apparatus may be configured to have a relatively narrow (low) spacer profile for inserting into (and/or removing from) tissue, while the spacer profile may be expanded (or allowed to expand) once in the tissue. This configuration may enhance the ease of inserting/removing the apparatus by changing the outer diameter of the spacer.

For example, the distal electrode may be translated distally and/or proximally to the proximal electrode to adjust the circumference (diameter) of the spacer. The spacer may be any of the spacers described herein, including spacers that are formed of an elastomeric material that may radially expand when compressed longitudinally and may radially collapse when pushed or stretched longitudinally. Thus, the apparatus may be inserted with a constant or relatively low radial size while the spacer (which may be, e.g., an insulating spacer) between the electrodes is the same diameter as the electrodes, but once the electrodes are in place the relative spacing between the proximal and distal electrodes may be adjusted to compress the spacer, so causing it to bulge outward which may create an intimate contact with the tissue.

One or more spacers may be used. In some examples the spacer may be formed of a conductive material or alternatively, an insulating material, such as a polymeric material, and may be attached to either or both the inner and outer elongate members. Alternatively in some examples the spacer may remain a relatively constant size (e.g., length and outer or inner diameter, as shown in FIG. 5B.

FIG. 5C illustrates another example of an embodiment of apparatuses described herein, whereby the distance between proximal and distal electrodes may be modified. Expanding on the exemplified treatment tool of, e.g., FIGS. 3E-3H, FIG. 5C illustrates a treatment tool comprising a proximal electrode 360, distal electrode 380, spacer 370 positioned between the proximal and distal electrodes, with the proximal electrode/spacer assembly retracted to expose vacuum outlet/inlet and/or infusion outlet/inlet 311. In the illustrated example, spacer 370 moves with the proximal electrode 360 when the proximal electrode/spacer assembly is retracted or pulled back to expose vacuum outlet/inlet and/or infusion outlet/inlet. In alternative examples, spacer 370 may be associated with the distal electrode 380. In such an embodiment, proximal electrode 360 may be retracted, exposing a vacuum outlet/inlet and/or infusion outlet/inlet 311 that may be located at the proximal (vs. distal, as shown) end of the spacer. As shown in FIG. 5C, distal electrode 380 may comprise a proximal alignment member 381 described in more detail in reference to FIGS. 5D and 5E.

FIG. 5D illustrates an example of a treatment tool similar to FIG. 5C. Visible in FIG. 5D, one or more vacuum and/or infusion lumens 373 that may span or pass through the proximal electrode 360, spacer 370, or both. The alignment member 381 of the distal electrode 380 may be configured to fit within the lumen 373 when the distal electrode 380 is connected to the spacer 370. The alignment member 381 may have a ramp-up or other alternative shape to assist in proper alignment of the distal electrode with the spacer when they are separated and reconnected during the use. Also visible in FIG. 5D is vacuum outlet/inlet and/or infusion outlet/inlet 311. Vacuum outlet/inlet and/or infusion outlet/inlet 311 can serve as the distal end or opening to one or more vacuum and/or infusion lumens 373 positioned within the proximal electrode 360 and electrode spacer (illustrated as having a single lumen) to facilitate aspiration of material (e.g., cell/tissue sampling), dispensation (e.g., therapeutic agent delivery), or both. In alternative examples (not shown), two or more lumens may be present. In some implementations, such lumens may be concentrically aligned, or may be positioned side by side, or at least some of the lumens may be concentrically aligned with one another while one or more other lumens may be positioned side by side relative to the concentrically aligned lumens. In some examples, any such lumen may have any suitable diameter. For example, one, some, most, or all lumens may comprise the same inner diameter, outer diameter, or both. In other examples, two or more lumens may comprise a different inner diameter, outer diameter, or both. Further, in some implementations one or more lumens may have substantively the same diameter along their respective length or a portion of the length, while other lumens may have variable diameter (wider or narrower portions) along the length or a portion of the length of such lumen.

FIG. 5E provides a cross-sectional view of the treatment tool as illustrated in FIG. 5C with the alignment member 381 of the distal electrode 380 positioned at the distal end of the spacer for connecting to the spacer. Distal electrode 380 extends beyond and outward from the vacuum outlet/inlet and/or infusion outlet/inlet 311, while distal electrode alignment member 381 ensures that if the proximal electrode and electrode spacer are advanced toward the distal electrode, the distal electrode will be appropriately aligned to re-mate. Distal electrode alignment member 381 may comprise a narrowed, sloped, or ramped design to center the spacer about the distal electrode during use.

As described in reference to FIG. 3E, an infusion or aspiration outlet 311 facilitates access to an interior lumen for aspiration, biopsy, or infusion/dispensation (thus, e.g., serving as a dispensation port), or any combination of the above.

Any of these apparatuses may include a lock or securing mechanism to lock or hold the spacing between the first and second electrodes. In some examples the lock may be coupled to the length adjuster. In some examples the lock may be on the handle. The lock may be coupled to the first (e.g., outer) and/or second (e.g., outer) elongate members. The lock may prevent actuation of the length adjuster.

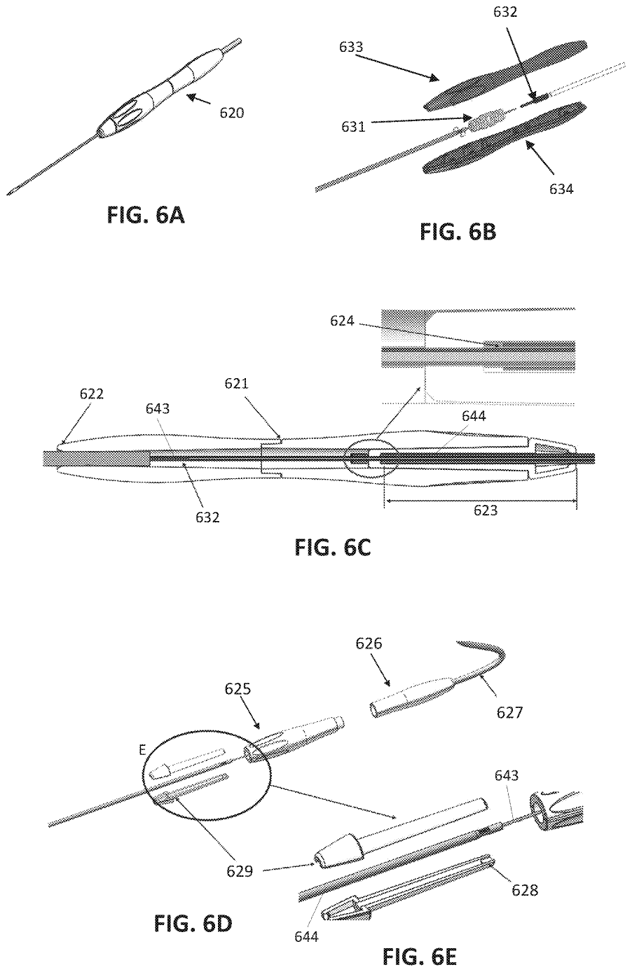

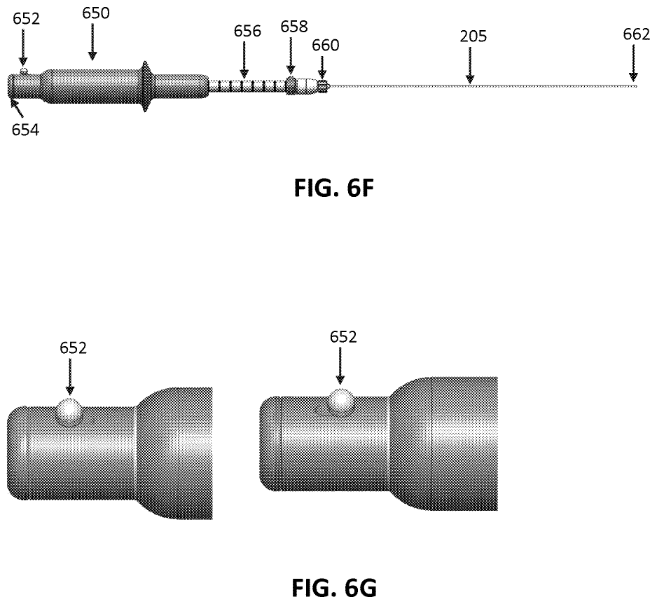

Various designs of the handle may be used in the tools, e.g., percutaneous or endovascular treatment tools, described herein. For example, the handle may have a cylindrical or a pencil shape. FIGS. 6A-6E show examples of handles for treatment tools as described herein. In some examples the handle may be a pencil-style handle 620, as shown in FIG. 6A. In general, the handle may act as an interface between the connection to the pulse generator (e.g., FIG. 1) and the electrodes of the treatment tool, and may be configured to securely transition between the cabling input from the pulse generator and the applicator (e.g., treatment tool) distal end which interfaces with the target tissue. In particular, the handle may be configured to couple the electrodes at the distal end region of the treatment tool with the high-voltage electrical input from the pulse generator in a manner that prevents harm or risk of shock to the user who may hold or operate the treatment tool by manipulating the handle. Connections between the cabling to/from the pulse generator and the electrodes at the distal end region of the treatment tool may include electrical insulation and/or isolation, including reducing or eliminating insulation creepage, which may be particularly important when using relatively high voltage, sub-microsecond pulses. In general, the handle 620 may be formed of an insulating material (e.g., a polymetric material) that may be configured to secure the cable and the internal electrical connectors to maintain internal and external creepage distances to prevent or reduce the risk of harm to the user. For example, in FIG. 6A, the handle is substantially hollow and includes internal channels and/or ribs to secure an end of the cable to/from the pulse generator and/or the inner elongate member and outer elongate members which may be coupled to and/or include the first and second wires for making electrical connections between the pulse generator and the electrodes, as mentioned above. The internal structure of the handle may include one or more insulating baffles to increase the creepage distance, e.g., between the inner elongate member (forming, coupled to or enclosing the second wire) and the outer elongate member (forming, coupled to or enclosing the first wire).

The handle may be reusable or disposable. As shown in FIG. 6B, in some examples the handle may comprise a top handle half 633 and a bottom handle half 634 configured to permanently or releasably couple together. As shown in the exploded view of FIG. 6B, the handle may also comprise a baffle 631 and an insulated connection 632 to the cabling to connect to the pulse generator. In FIG. 6B this insulated connection 632 is a heat-shrink connection which may act as a barrier to help achieve additional creepage length and clearance distances. Baffle 631 may be configured to increase a minimum clearance distance between the conductive parts that may further reduce risk of current leaking which may otherwise damage the apparatus and/or risk harm to the user. The minimum clearance distance, as used in the present disclosure, may indicate the shortest distance that avoids current leakage in the air or along an insulating material surface path. In other words, the minimum clearance distance can include a distance that is the greater of the following two distances: 1) a shortest distance or path that prevents current leakage between two conductive parts measured along any surface or combination of surfaces of an insulating material, and 2) the shortest path in air between two conductive parts that prevents current leakage. A “creepage distance” includes a shortest distance that prevents current leakage (e.g., in some examples, arcing) along the surface of the insulating material between two conductive parts, as defined by the International Electrotechnical Commission (IEC), or as otherwise known in the art. It can include the surface distance from one conductive part to another conductive part, or an area accessible by a user. “Air clearance” includes the shortest path that prevents arc in air between two conductive parts as defined by the IEC, or as otherwise known in the art. It can include the uninterrupted distance through the air or free space from one conductive part to another conductive part or an area accessible by a user.

In the example shown in FIG. 6B, the baffle 631 inside the handle 620 increases the minimum clearance distance as the length along the surface of the baffle increases. The baffle 631 can create, for example, 20 mm to 80 mm of the minimum clearance distance, 30 mm to 70 mm of the minimum clearance distance, 40 mm to 60 mm of the minimum clearance distance, etc. In FIG. 6B, the heat shrink insulation 632 can cover high voltage wire and solder joints to prevent internal creepage. The baffle 631 and similar structures for increasing minimum clearance distance may be formed as described in various examples of the co-owned Patent Publication US 2019/0269904 A1, which is incorporated herein for reference.

FIGS. 6C-6E illustrate another example of a handle for a treatment tool as described herein. In FIG. 6C the handle includes a distal handle section and a proximal handle section that are coupled together, e.g., at a bonding region 621. The proximal end of the handle is secured, e.g., by an adhesive 622, to the cabling configured to couple to the pulse generator. The cabling may include two or more wires that are safely distributed within the handle to the electrodes at the distal end region of the device (not shown). For example, in FIG. 6C, insulating heat shrink material 632 may cover a first high-voltage wire from the cabling that is coupled to the inner elongate member 643. This insulating heat shrink material, as well as solder joints between the wire and the inner elongate member, may prevent internal creepage and clearance issues, as described above. The handle may also include one or more (or a plurality) of internal supports or structures that are configured to secure the elongate members (e.g., the inner 643 and outer 644 elongate members) within the handle and may help isolate and prevent creepage of leak current. The elongate members may be insulated along their length, and this insulation may extend into and within the handle. For example, the outer member or shaft 644 may include outer shaft insulation 624 that extends into the handle to prevent creepage. In addition, the handle may be dimensioned to include additional minimum clearance to reduce current leakage (creep) from the connection between the wires and the elongate members. For example, in FIG. 6C, the spacing 623 between the distal end of the handle and the end of the outer elongate member, where the electrical connection to the first wire may be made, may be greater than 48 mm, so that the external minimum clearance distance is greater than 48 mm.

FIG. 6D shows an exploded view of the handle of FIG. 6C. In this example the proximal handle section 626 is shown separated from the distal handle section 625. As mentioned, the proximal end couples to cable 627 configured to connect to the pulse generator. In the example shown in FIGS. 6C-6E the handle also includes elongate shafts 644, 643 and one or more shaft locators 629 that may help secure, and in some examples electrically isolate, the elongate shafts within the inside of the handle. FIG. 6E shows an enlarged view of the encircled section E with the shaft locator 629 of FIG. 6D. The shaft locator 629 includes an internal channel in which the shaft(s) 644, 643 of the tool may sit. The shaft locator also includes one or more internal boss 628 that may mate with a seating region (e.g., notch) in the shaft(s) to limit or prevent (e.g., constrain) movement in the longitudinal (axial) direction.

FIGS. 6F and 6G illustrate additional examples of handle(s) of apparatuses of the present technology. FIG. 6F illustrates a handle with an introducer shaft and treatment tip region. Handle 650 in this example comprises an aspiration valve control 652. An aspiration valve control 652 may have any design or configuration suitable for controlling the activation or termination of aspiration function(s) of an apparatus (such as, e.g., for the collection of material through a treatment tool, e.g., through a vacuum outlet/inlet and/or infusion outlet/inlet 311 illustrated elsewhere herein). For example, an aspiration valve control may be a button, switch, lever, dial, etc. In the provided example of FIG. 6F, aspiration valve control is provided as a spring-loaded levered button, spring-loaded to be in a closed (auto close) position. It is contemplated that any suitable activation/deactivation (on/off) control could be used. In use, a user may apply sufficient force to the aspiration valve control to place the device in an active or “on” state from an inactive or “off” state. In the exemplified embodiment, a user may move the levered button to an active position, thus applying aspiration (vacuum) force through the apparatus, e.g., to the vacuum outlet/inlet and/or infusion outlet/inlet 311 (not shown), for example, to collect cells/tissue from the target treatment site. An exemplary aspiration valve control 652 is illustrated again in FIG. 6G, illustrated as being in an, e.g., active or “on” position (left) and a default closed or “off” position (right). Readers can appreciate that such a control mechanism may be any other suitable control mechanism capable of activating a vacuum force. According to various embodiments, a mechanism for defaulting the aspiration valve control to an inactive or “off” position is incorporated, for example, spring loading the control. In some implementations, devices of the present disclosure may comprise a mechanism forcing a user to maintain sufficient force on an aspiration valve control to maintain application of vacuum pressure sufficient to collect material from a treatment site.

In addition to the aspiration valve control or alternatively a handle may comprise an infusion valve control. An infusion valve control may have any design or configuration suitable for controlling the activation or termination of infusion function(s) of an apparatus (such as, e.g., for the infusion of material through a treatment tool, e.g., through a vacuum outlet/inlet and/or infusion outlet/inlet 311 illustrated elsewhere herein). For example, an infusion valve control may be a button, switch, lever, dial, etc. In certain aspects, an infusion valve control may be a spring-loaded levered button, spring-loaded to be in a closed (auto close) position. It is contemplated that any suitable activation/deactivation (on/off) control could be used. In use, a user may apply sufficient force to the infusion valve control to place the device in an active or “on” state from an inactive or “off” state to initiate infusion of a material, e.g., a fluid, e.g., saline or therapeutic agent such as a drug (or other treatment compositions or medications described herein) at the target treatment site. Readers can appreciate that such a control mechanism may be any other suitable control mechanism capable of activating an expulsive/expulsion force. In some implementations, devices of the present disclosure may comprise a mechanism forcing a user to maintain sufficient force on an infusion valve control to maintain application of expulsion/expulsive pressure sufficient to deliver material to a treatment site.

Returning to FIG. 6F, the apparatus further comprises a cable and luer lock 654. Cable and luer lock 654 may operate as a connection point to other apparatus element(s), such as fluid tubing, collection containers (e.g., a syringe or the like) (not shown). This may be beneficial, for example, for collection of aspirated material so that it may be removed from the apparatus and analyzed. In such an embodiment, a user can maintain the apparatus in place, e.g., maintain the treatment tool positioned at the treatment site within a patient, while collecting, removing, and analyzing material collected therefrom. Analysis may comprise, for example, characterizing the collected material to facilitate decision making related to target site treatment.

The apparatus illustrated in FIG. 6F further comprises a tip deployment gauge 656 and a tip deployment stop 658 for aiding or participating in the deployment or manipulation of the introducer shaft, the treatment tool tip, or one or more other apparatus components. One or more luer connectors 660 may be present to aid in the maintenance of a stable position of one or more apparatus components (such as, the introducer shaft, treatment tool/treatment tip, or both).

The apparatus is further illustrated as comprising introducer shaft 205 and treatment tool tip region 662 (inside the introducer shaft). As illustrated, tip region 662 is positioned fully within the introducer shaft 205 and not visible. Introducer shaft 205 may comprise any suitable length, for example, 800 mm or greater, 1000 mm or greater, 1400 mm or greater, 1500 mm or greater. Introducer shaft 205 may comprise any suitable outer diameter, such as an outer diameter of about 10 mm or less (“≤~10 mm”), ≤~5 mm, ≤~4 mm, ≤~3 mm, ≤~2.9 mm, ≤~2.8 mm, ≤~2.7 mm, ≤~2.6 mm, or, e.g., ≤~2.5 mm.

### Arc Mitigation Solutions and Examples

According to another aspect of the present disclosure, any of the apparatuses (e.g., devices, systems, etc.) described herein may be configured to prevent or at least reduce arcing between the electrodes of the apparatus of the present disclosure. As discussed above in reference to FIGS. 2 and 3A-3B, this may be achieved at least in part by using a vacuum to remove air from around (e.g., between) the electrodes or in reference to FIGS. 3C and 3D by coupling the spacer within the distal end region without adhesive. In some examples this can be achieved without the use of vacuum or in addition to the use of vacuum, e.g., by implementing various features, alone or in various combinations, as described herein.

### Reduction Of Peak Electric Field

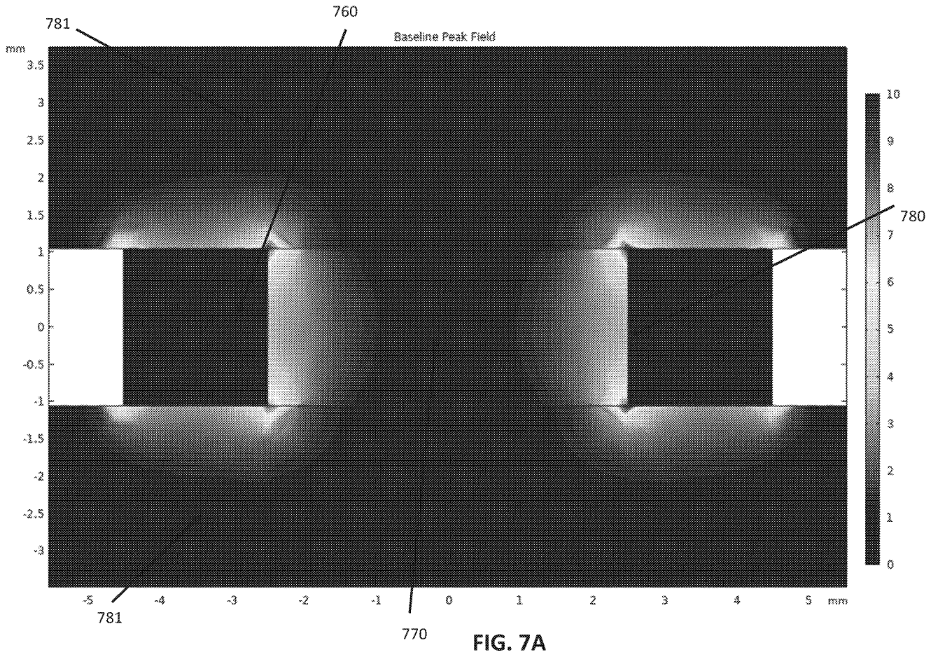

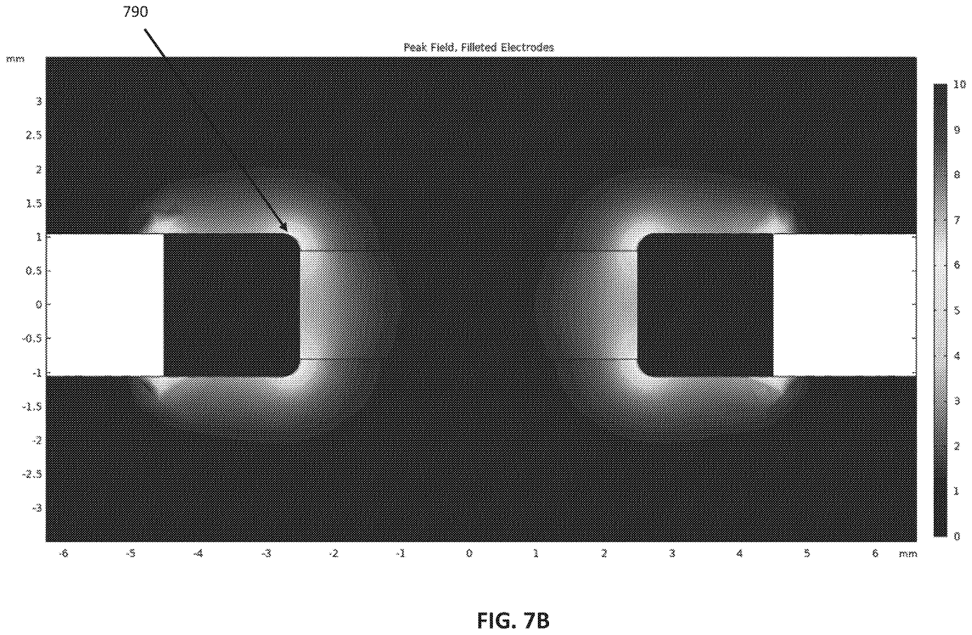

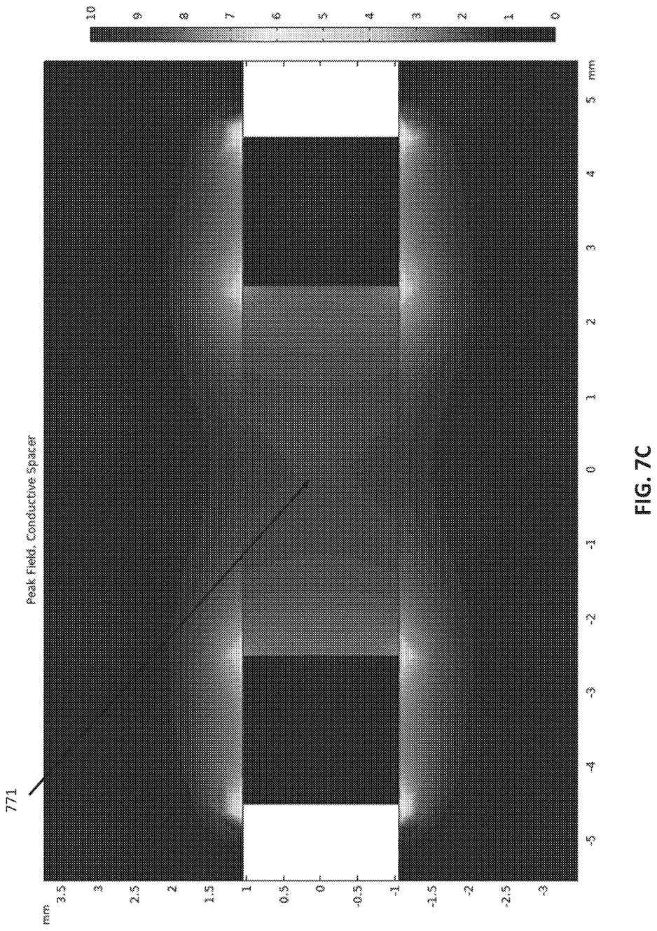

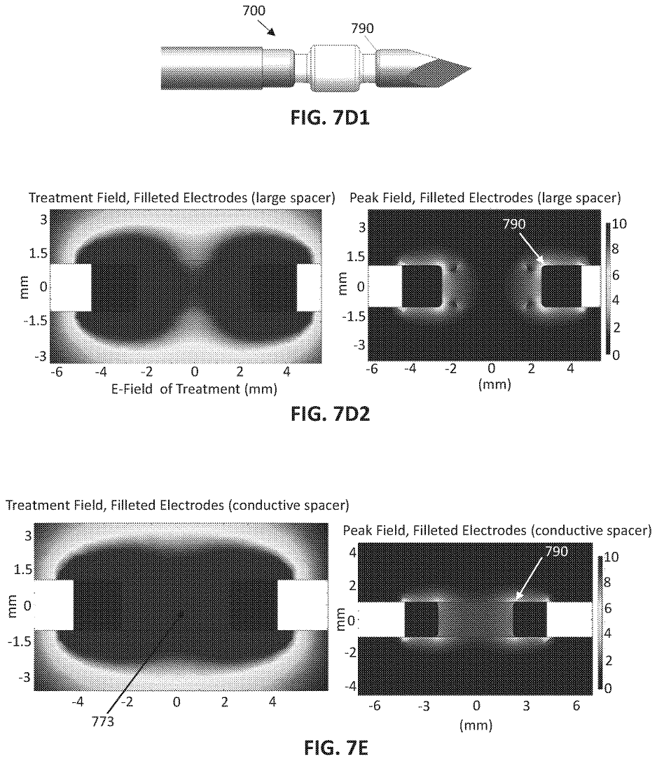

In various applications it may be important to maintain the high level of the treatment electric field for successful treatment; however, at the same time the peak electric field may be reduced. Typically, a peak electric field may vary depending on the type of edge of the electrodes in the tip region. For example, the peak electric field usually appears near edges of each electrode. It may be helpful to reduce the peak electric field since the higher peak electric fields are more likely to arc between electrodes. Electrode design can cause the peak electric fields to be different for the same voltage potential. Reducing the peak fields for a given potential can reduce the risk of arcing. The electric fields can be concentrated by sharp edges of the electrode and abrupt changes in conductivity. Therefore, according to some examples of the present disclosure peak electric field can be reduced by rounding corners, e.g., edges, of the electrodes (providing, for example, fillets) as described in reference to FIGS. 7B and 7D below. Another feature that allows the reduction of peak electric field according to the present disclosure is providing a conductive spacer as described in reference to FIGS. 7C and 7E below. An additional feature that allows the mitigation of arcing between the electrodes according to the present disclosure comprises providing a spacer (either insulative or conductive) that has at least a portion with a circumference or diameter that is larger than a circumference or a diameter of the electrodes. Such a feature is demonstrated in FIG. 7D1.

According to one example of using the devices and methods of the present disclosure, nanosecond pulsed electric treatment was performed on the thyroid glands of four Yorkshire pigs using a treatment tool as described herein, having an electrode array inserted through a small incision in the neck and into one side of the thyroid lobe. Histological assessment of the treated tissue was performed at 0, 2, 8, and 30 days post treatment to determine the impact of the treatment on the parenchymal and stromal portions of the gland. Resulting intense Caspase-3 staining throughout the treatment zone at day 0 indicates that the treatment performed can initiate programmed cell death in a spatially defined region. At 30 days, pronounced parenchymal loss was evident within the treatment zone with minimal inflammation, continued phagocytosis and collagen remodeling.

The results show that nanosecond pulsed treatment as described herein may be a useful, minimally invasive technique to treat, for example, benign thyroid nodules while sparing the surrounding normal thyroid tissue and reducing risk of collateral damage to nerves and vessels.

FIGS. 7A-7E show examples of the results of the COMSOL analysis of the electric fields distribution corresponding to the treatment tools with different features configured to reduce peak electric field according to an aspect of the present disclosure. FIG. 7A shows an example of the baseline electric field without curved edges (fillets), conductive spacer, or larger-diameter spacer features as described herein. Specifically, in the example of FIG. 7A, the electrodes 760, 780 have a diameter of 2 mm and are separated by an insulative 5 mm long spacer 770. As shown in FIG. 7A, the peak electric field in the thyroid tissue 781 was measured and used as a baseline to compare with the peak electric field using the tool configurations that incorporate various features described herein. As mentioned, the peak electric field appeared at the edges of the electrodes where an abrupt change in the conductivity occurs since the electrodes 760, 780 are in contact with the insulative spacer 770 and the thyroid tissue 781.

FIG. 7B shows an example of the electric fields of the treatment tool having fillets (rounded corners). As seen in FIG. 7B, adding a rounded corner 790 can reduce the peak electric field, for example, by 15% to 30%, by 20% to 25%, depending on the type of the tissue. In this example, with the same spacing of 5 mm between the electrodes and the same electrode outer diameter of 2 mm in the thyroid tissue, the peak electric fields showed about 19% reduction compared to the baseline of FIG. 7A. Any appropriate radius of the curvature for the fillet 790 may be used, for example, if the electrode has a thickness of t, the radius of curvature may be larger than about t/8 (e.g., larger than about t/7, larger than about t/6, larger than about t/5, larger than about t/4, larger than about t/3, between about t/8 and about 4t, between about t/8 and 2t, etc.). In general, a larger radius of curvature may be preferred. For example, in some apparatuses the radius of curvature of the curved edge (fillet) may be, e.g., between about 0.1 mm to 0.5 mm for certain dimensions of the electrodes.

FIG. 7C shows an example of electric fields of the treatment tool having a conductive spacer between the electrodes. In this example, the conductive spacer 771 has the same circumference as the electrodes. Conductive spacer 771 may be made from any suitable conductive material having a desired conductivity. For example, conductive spacer may be made from a hydrogel, a conductive adhesive, a conductive gel, a conductive silicone, a urethane rubber, carbon nanotubes, or any combination thereof. The desired conductivity of the conductive spacer 771 may be selected, for example, based on the conductivity of the tissue being treated or conductivity of the skin/tissue at the percutaneous introduction of the apparatus. For example, the conductivity of the conductive spacer may be substantially the same as conductivity as the treatment area, in some examples, up to ten times (10×) the conductivity of the treatment area, and up to approximately one hundred times (100×) the conductivity of the treatment area. In some embodiments, the conductivity of the conductive spacer may vary throughout the conductive spacer 771. For example, the conductive spacer may have zones that have different conductivity and/or there may be a gradient of conductivity within the conductive spacer. In the implementations according to the present disclosure, selecting the electrode assembly may be based on the conductive spacer having a conductivity that is less than, greater than, or equal to a conductivity of a tissue of the treatment area. In some examples, the method may include selecting the electrode assembly based at least in part on a conductivity of the conductive spacer. In other examples, selecting the electrode assembly may be further based at least in part on a size of the electrode assembly and a size of the treatment area. In some implementations, the method of treating a tissue with a pulsed electric field may include selecting the voltage to be applied to the treatment area based at least in part on a conductivity of the conductive spacer.

The consistency of the conductive spacer 771 may be solid, compressible, or gelatinous yet firm enough to maintain shape and position within electrode assembly. The conductive spacer 771 alone can reduce the peak electric field by 25%-50% compared to the electrodes with the insulative spacer since the conductive spacer can relieve the abrupt change in the conductivity at the edge of each electrode. Under the same condition as FIG. 7A in the electrode circumference and the spacing between the electrodes, in this example the peak electric field demonstrated about 41% reduction compared to the baseline of FIG. 7A.

FIG. 7D1 illustrates a treatment tool 700 having fillets 790 on the electrodes and an insulative spacer with a circumference larger than the circumference of the electrodes. FIG. 7D2 illustrates the corresponding electric fields. The larger circumference or diameter on at least the portion of the spacer increases tissue contact pressure which, in turn, also mitigates the risk or arcing. Under the same condition as FIG. 7A in the electrode circumference and the spacing between the electrodes in the thyroid tissue, this example shows about 21% reduction in the peak electric field compared to the baseline of FIG. 7A.

FIG. 7E illustrates electric fields corresponding to the treatment tool having a conductive spacer with the same circumference (e.g., OD) as the electrodes. In this example, the peak electric field at electrode/conductive spacer junction due to the combination of the fillets and the conductive spacer is reduced by about 43% compared to the baseline of FIG. 7A. Also, as shown in the left image of FIG. 7E, conductive spacer 773 increases treatment field in the middle between the electrodes, which may provide further advantage of more uniform treatment in the area in the middle between the electrodes.

As mentioned above, any of the treatment tool configurations shown in FIGS. 7A-7E may be implemented with a treatment length adjustment feature as described above. Depending on the particular configuration, any appropriate modifications may be made to the length adjuster as will be understood by those skilled in the art. In some examples when a conductive spacer is used, it may be desirable to use two or more conductive spacers each adjacent to the respective first and second electrodes, and these conductive spacers may be separated by a gap that allows for a length adjustment and sliding in and out.

### Increasing Hoop Stress

Good contact between the tissue to be treated and the material between the electrodes (e.g., a spacer) may be important for arc mitigation or prevention. Any air path or fluid path directly from one electrode to another electrode can provide an arc path. Therefore, increasing the hoop stress on the tissue may provide a better seal against the spacer. The hoop stress may be introduced by stretching the tissue rather than cutting the tissue while inserting the tip region of the tool into the target tissue. Some examples of the configurations of the tip region (including the distal electrode) for increasing the hoop stress according to the present disclosure include: (i) increasing circumference (e.g., diameter) of a spacer relative to the circumference (e.g., diameter) of the electrode to force the tissue to stretch in that zone and make a good contact with the electrodes and the space between the electrodes, (ii) decreasing the cutting circumference (e.g., diameter) of the distal end of the tip region to make the cutting section smaller than the electrode circumference/diameter, and/or (iii) using a tip with no cutting edges, where the tip insertion only stretches the tissue. Finally, the tip may avoid abrupt changes in the angle of the surface (particularly in otherwise flat surfaces) that may introduce a gap between the tissue and the surface.

FIGS. 8A-8F show examples of the configurations of a distal end of the tip region (distal electrode) according to the present disclosure. The treatment tools of the present disclosure may also be referred to as the percutaneous needle electrode. FIGS. 8A-8B show a front view and a side view, respectively, of an example of a trocar-shaped distal end or tip. The trocar tip 882 penetrates the tissue by cutting the tissue. Three cutting edges 881 may reach to the full circumference of the needle tip, which means that the trocar tip 882 is more effective to cut tissue rather than stretch or expand the tissue. While a trocar tip may not be optimal on its own for the purposes of the present disclosure, it may still be useful when combined with other features of the present disclosure, such as spacers with the increased circumference, conductive spacers, or rounded corners of the electrodes. Further, in some examples (described below) the angles between the faces of the trocar may be configured to minimize the introduction of gaps or spaces with the tissue.

FIGS. 8C-8D show a front view and a side view, respectively, of an example of a conical distal end or tip. The conical tip (“cone” or “pencil”) penetrates the tissue by expanding the tissue rather than by cutting it. As shown in FIGS. 8C-8D, the conical tip 884 does not have the cutting edges (as shown by a smooth surface 883) found in the trocar tip. The conical tip may be effective to stretch or expand the tissue when the tip region with the distal conical electrode is inserted into the target tissue. Therefore, the conical tip may help to make good contact between the tissue and the tip region. However, a relatively higher pressure may be needed when a conical tip is inserted into the tissue since there is no cutting edge in the conical tip.

FIGS. 8E-8F show a front view and a side view, respectively, of an example of a hybrid (e.g., pencil-trocar) distal end or tip. The hybrid tip 889 may be a combination of the cone/pencil tip and the trocar tip, and it is especially useful in obtaining good contact with the tissue and increasing hoop stress. The hybrid tip penetrates the tissue by only initially cutting the tissue to, for example, a circumference or diameter smaller than the full outer circumference or diameter of the tip, which helps with the initial insertion of the tool. Then the hybrid tip only expands the tissue to the full circumference by stretching. The cutting portion of the circumference may extend along the length of the tip from the distal pointed end to a proximal length at which the circumference is about 5% to about 50% of the full circumference of the tip of the electrode (e.g., at the proximal end of the tip) but is not limited to these ranges. As shown in FIG. 8E, the front view of the hybrid pencil-trocar tip shows the conical non-cutting section 885 and the trocar style cutting section 886 with 3 cutting edges 887. The cutting edge portion of the tip ends at 888, at about 40% of the length of the tip, at which point the tip has a smaller circumference than the full circumference of the distal electrode. The hybrid tip 889 can be effective to easily penetrate but then stretch or expand the tissue when inserted into the target tissue compared to either the trocar type tip or the cone tip (e.g., conical tip). Due to its cutting edges 887, it requires less pressure than the cone tip when inserted into the tissue, which makes it easier to operate.

In general, a hybrid tip as described herein may include a distal cutting portion having one or more cutting edges (e.g., blades), such as a 3-sided trocar as described above, and a larger-circumference (larger outer diameter) smooth, flat or otherwise non-cutting surface that is proximal to the cutting edges. The non-cutting surface that is more proximal may therefore stretch and expand the tissue as the tip is advanced. Although conical (cone) shaped proximal regions may be used, other non-conical shapes may also be used, including other flat or curved surfaces. As described above, shapes that transition gradually during expansion of the tissue so as not to create gaps between the tissue and the electrode tip may be desirable, to prevent arcing.

In any of the tips described herein the tip profile may be configured to prevent abrupt changes in the angle between the wall(s) of the tip and the tissue, which might otherwise introduce gaps between the tip and the tissue that may allow for arcing and/or poor electrical contact. For example, in reference to the hybrid tips described in FIGS. 8E-8F, the transition between the flat (e.g., monotonic) wall(s) forming the trocar region and the conical region may be sufficiently shallow (e.g., change at an angle of about 20 degrees or less, 19 degrees or less, 18 degrees or less, 17 degrees or less, 16 degrees or less, 15 degrees or less, 14 degrees or less, 13 degrees or less, 12 degrees or less, 11 degrees or less, 10 degrees or less, etc.) so that the tissue remains in contact with the tip. If the wall transitions from a less steep region of the trocar to a steeper region of the cone, then the transition may act more like a step and the tissue may lose contact in this region, which could cause corona or arcing between the tissue and the metal electrode tip. Alternatively, in some examples it may be beneficial to include a curved transition between different angled regions of the tip.

FIGS. 9A-9F show various examples of the tip region configurations of the treatment tool of the present disclosure. Various combinations of the novel features of the present disclosure are shown. To increase the hoop stress, a hybrid tip (electrode) or a spacer with a greater circumference than the shaft circumference may be used. FIG. 9A illustrates an example of a tip region having a hybrid tip 982 and a spacer 971 with the same circumference as the shaft and the electrodes 961, 982. The hybrid tip 982 electrode includes a trocar style cutting distal section and a smooth non-cutting proximal section so that the proximal circumference (e.g., diameter) at the cutting edge end 981 is smaller than the full diameter of the shaft. The spacer 971 may be an insulative spacer or a conductive spacer which has the same circumference as the shaft (electrodes). Spacer 971 connects to the first electrode 961 which is proximally insulated by the exterior insulation 951. The tip region in FIG. 9A may increase the hoop stress by the hybrid tip to make good contact between the tissue and the tip region.

FIG. 9B illustrates an example of a tip region having a distal electrode 983 (trocar tip) and a spacer 972 with a circumference that is greater, at least along a portion of a length of the spacer, than the circumference of the electrodes 983, 961. The spacer may be an insulative spacer or a conductive spacer which has a greater circumference than the electrodes. The greater circumference of the spacer can force the tissue to stretch in that zone thereby introducing the hoop stress that may aid in mitigating/preventing arcing.

FIG. 9C illustrates an example of a tip region having a trocar tip 983 and a conductive spacer 973 with the same circumference as electrodes. This type of the tip region can decrease the peak electric field by using the conductive spacer 973 as explained in reference to FIG. 7C, above. FIG. 9D illustrates an example of a tip region having a pencil tip 985 and a conductive spacer 973 with the same circumference as electrodes. This type of the tip region can also decrease the peak electric field by introducing the conductive spacer 973 and further can force the tissue to stretch as the pencil tip 985 advances into the tissue, to mitigate/prevent arcing.

FIG. 9E illustrates an example of a tip region having a trocar tip 983 and a spacer 974 with a circumference that is greater (at least along a portion of a length of the spacer) than the circumference of the shaft where the electrodes are positioned; the electrodes include rounded edges (e.g., fillet) 991 at the edge facing the spacer 974. The spacer 974 may be an insulative spacer or a conductive spacer. This type of the tip region can decrease the peak electric field by introducing the fillet 991 as explained in reference to FIG. 7B and further can increase the hoop stress by using the greater circumference of the spacer than the shaft which can force the tissue to stretch in that zone to mitigate and/or prevent arcing. FIG. 9F shows an example of a rounded edge (e.g., fillet) 991 where the edge of the electrode 962 facing toward the spacer 974 is round. The first electrode 962 is insulated by exterior insulation 951.

The various tips and tip region configurations disclosed herein for use in exemplified embodiments of apparatuses of the present disclosure can be selected based on the particular application/procedure. In certain respects, the size will vary based upon the procedure to be performed. In some examples tip regions may comprise one or more lumens to facilitate aspiration or delivery of materials as described elsewhere herein. In certain embodiments, tip regions having 18 G size may be used, for example, to facilitate biopsy (ies), while in alternative embodiments, a smaller size, such as 23 G or higher, may be appropriate for smaller anatomical environments or, e.g., applications associated with fine needle biopsy.

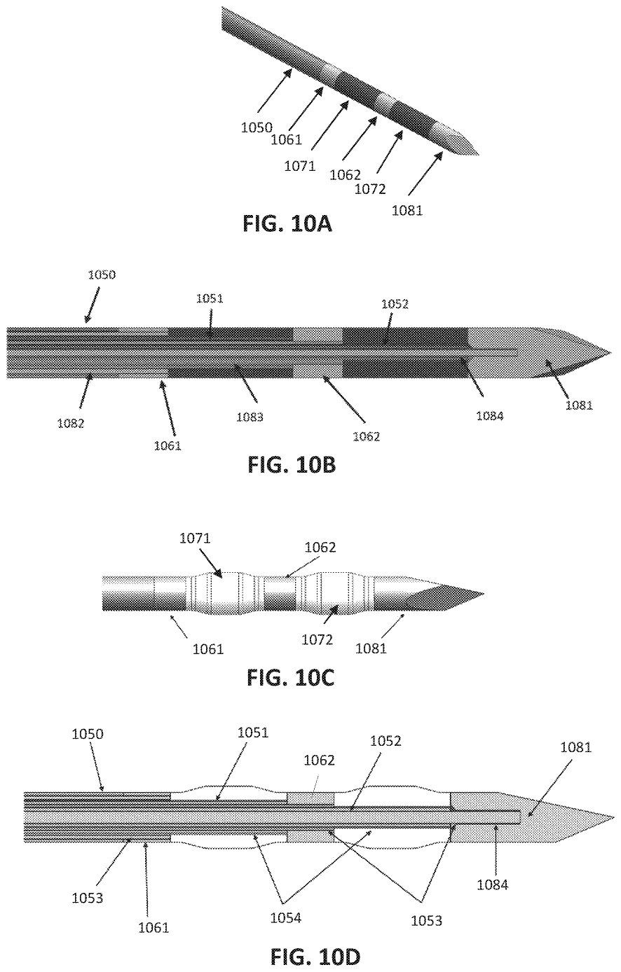

FIGS. 10A-10D show examples of tip regions of a treatment applicator with three electrodes. FIG. 10A illustrates a perspective view of a tip region with three electrodes. FIG. 10B illustrates a cross-sectional view of the tip region with three electrodes shown in FIG. 10A. The electrodes may be bipolar or monopolar. When the tip region is configured to deliver energy in a bipolar manner, a proximal electrode 1061 and a tip electrode 1081 may be positive electrodes and a middle electrode 1062 may be a negative electrode, or the proximal electrode 1061 and the tip electrode 1081 may be the negative electrodes and the middle electrode 1062 may be the positive electrode. Both a proximal spacer 1071 and a tip spacer 1072 may be an insulative spacer or a conductive spacer, or one of the proximal spacer 1071 and the tip spacer 1072 may be the insulative spacer and the other of the proximal spacer 1071 and the tip spacer 1072 may be the conductive spacer. An exterior insulation 1050 may circumferentially insulate the first (outer) hypotube 1082 which connects to the proximal electrode 1061 so that the proximal electrode 1061 is only exposed over the outside region to make contact with the tissue. As shown in FIG. 10B, an inner (e.g., “middle”) insulation region 1051 can insulate the inner surface of the first hypotube 1082 and the outer surface of a second hypotube 1083 from each other. The second hypotube 1083 may connect to the middle electrode 1062 to make electrical contact between the electrode and the pulse generator (and therefore the tissue). A second inner insulation 1052 may insulate the inner surface of the second hypotube 1083 and the outer surface of the third hypotube or rod 1084 (e.g., shown as a rod in FIG. 10B) from each other. The third hypotube or rod 1084 connects to the tip electrode 1081. As mentioned, the tip electrode may be any type of tissue-penetrating tip (e.g., needle) such as a trocar tip, pencil tip, or hybrid tip. Each of the exterior insulation 1050, the middle insulation 1051 and the inner insulation 1052 may be the same or a different insulator and have the same or different thicknesses. For example, in FIG. 10B, the exterior, and both inner insulation layers may be formed of a polyimide insulation and have a thickness, for example, of between about 0.0005-0.05 inches, 0.001-0.01 inches, or 0.005 inches. The first hypotube 1082 may have a thickness, for example, of between about 0.005-0.5 inches, 0.01-0.1 inches, or 0.065 inches. The second hypotube 1083 may have a thickness, for example, of between about 0.005-0.5 inches, 0.01-0.1 inches, or 0.036 inches. The third hypotube or rod 1084 may have a diameter or thickness, for example, of between about 0.005-0.5 inches, 0.01-0.1 inches, or 0.020 inches. The proximal electrode 1061 and the middle electrode 1062 may be welded to the first hypotube 1082 and the second hypotube 1083, respectively. The apparatus with the three electrodes may enable to treat a larger area of the target tissue.

FIG. 10C shows another example of a tip of a treatment tool including three electrodes: a first (proximal) electrode 1061, a second (middle) electrode 1062, and a third (distal) electrode 1081. The third electrode may be configured as a tissue-penetrating tip electrode as described herein. In FIG. 10C, the tissue-penetrating tip electrode is configured as a trocar. As in the example shown in FIGS. 10A-10B, the first and third electrodes may be electrically coupled to provide an opposite polarity as compared to the second (middle) electrode. In other examples, the two electrodes may be electrically coupled to provide a voltage (or potential) differential between them and the third electrode may be used as a ground. In this example the first, second and third electrodes are separated by spacers (e.g., insulating or conductive spacers) that have a larger circumference than the OD of the adjacent annular electrodes.

FIG. 10D shows a sectional view through the tip of the treatment tool shown in FIG. 10C. The construction of the tip shown in FIG. 10D is similar to that shown in FIG. 10B, and includes an outer insulation 1050 (e.g., in one example, formed of 0.005″ polyimide that is bonded to the outer shaft). The third electrode 1061 is formed of 0.085″ OD stainless steel tubing that is soldered 1053 to the outer hypotube. A first inner insulation layer 1051 (e.g., formed of 0.005″ polyimide in this example) is bonded to the middle shaft, to which the middle electrode 1062 is also soldered 1053. The third, distal electrode (formed as a trocar tip) 1081 is soldered to the inner shaft 1084 (shown in this example as a stainless-steel rod having a 0.020″ OD). The inner shaft is also insulated by a second inner insulation layer (e.g., a 0.005″ polyimide insulator bonded to the inner shaft). A first spacer 1054 and a second spacer 1054′ are bonded to the insulation.

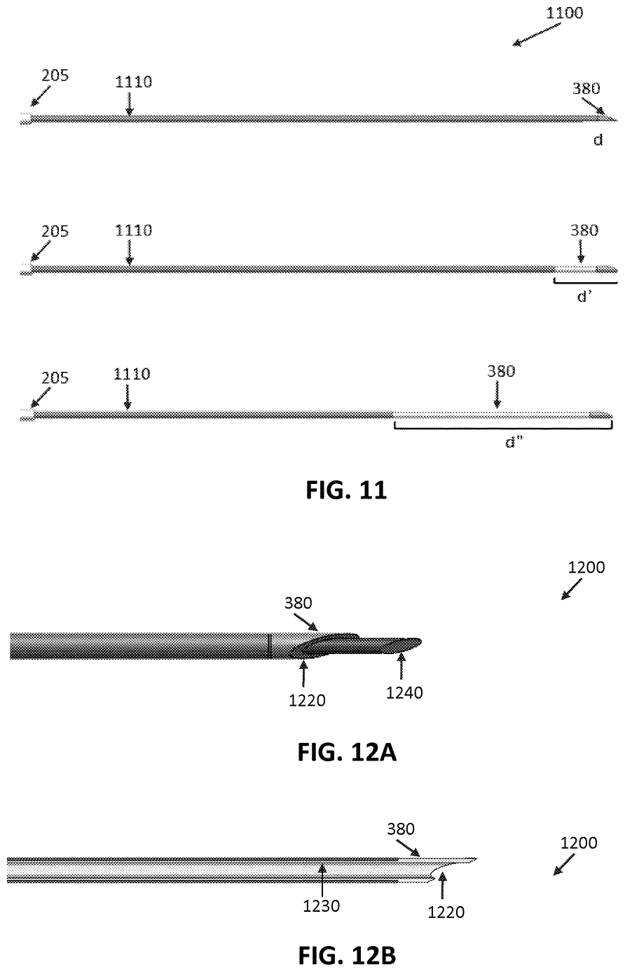

FIG. 11 illustrates an example of a tip region of the treatment device 1100 extending from an introducer shaft 205, through which the treatment device may be deployed. Treatment device 1100 comprises outer insulation 1110 and at least one treatment electrode, e.g., distal electrode 380. In some examples, outer insulation 1110 is movable with respect to one or more other device components. In this example, apparatus of FIG. 11 comprises a monopolar electrode, e.g., embodied as a tip electrode (or distal electrode) 380, having a tip design suitable for piercing tissue. A ground pad may be positioned somewhere on a patient to create the return path. A distal (tip) electrode may comprise a lumen, as shown by example in FIGS. 12, 13 and 14. An exposed length of the distal electrode 380 may be adjusted to control the size of the treatment area. Tip region of the treatment device 1100 may be movable in and out of introducer shaft 205 as needed. For example, during delivery of the apparatus 1100 to the target tissue the tip region may be within the introducer shaft. Tip region of the treatment device may extend from an introducer shaft, e.g., by 10 mm (1 cm), 20 mm (2 cm), 30 mm (3 cm), 40 mm (4 cm), or, e.g., 50 mm (5 cm), 80 mm (8 cm), 100 mm (10 cm) or more, when the tip electrode(s) is/are placed into a target site (e.g., lesion) for treatment by application of electrical energy. As shown in FIG. 11, outer insulation 1110 may be adjustable/movable to enable different length of exposure of the distal electrode of the tip region, and therefore allow to adjust the ablation zone. Outer insulation 1110 may be controlled to move back such that it covers all but a very tip (e.g., beveled) portion of the distal electrode 380 (FIG. 11, top) represented by (d). Also or alternatively, outer insulation 1110 may be movable to expose additional length of the distal electrode 380, for example, exposing distance d′ (FIG. 11, middle), or even further distance d″ exposing, for example, full length of the distal electrode 380 (FIG. 11, bottom). The outer insulation may be configured, for example, to slide to expose between 2 mm and 50 mm of the distal tip of the tip region. Such extension distance(s) may be modified by the user as applicable to the procedure or step in a procedure being performed.

FIGS. 12A and 12B illustrate an example of a tip region of an apparatus (treatment device) 1200 wherein a treatment electrode, e.g., distal electrode (e.g., a distal or tip electrode) 380 is configured as a piercing needle electrode comprising a fluid path/lumen 1230 passing through the treatment tip region and an opening 1220 (e.g., within a distal/tip treatment electrode) to the fluid path/lumen 1230 present in a distal electrode, wherein an optional stylet 1240 (top) may be optionally positioned within the fluid path. In this example, lumen opening 1220 within treatment electrode 380, and the fluid path/lumen 1230 facilitate both the application of electrical energy to a target site and, optionally, the aspiration of material (e.g., sampling of fluid, cells, tissue, etc.), the dispensation of a fluid or therapeutic agent (e.g., delivery of a chemotherapy drug, etc.), or both. In this embodiment, as shown elsewhere herein, treatment electrode (distal/tip electrode) 380 may be selectively extended at varying distances from an introducer shaft or an insulative layer.

In certain examples, treatment electrode (distal/tip electrode) 380 may have more than one lumen, e.g., two or more or three or more lumens. In some examples, each lumen may have a dedicated function, such as, e.g., aspiration of material, dispensation/delivery of material, and the like. In some examples, lumen(s) may have any configuration described elsewhere herein, such as, e.g., being concentric, non-concentric, having the same or different diameters, and the like.

In operation, a device user may wish to block lumen(s) during insertion of the device to keep it clean and clear. In such a scenario, as shown in FIG. 12A, an optional stylet 1240 may be used for positioning within a lumen, wherein the stylet may be removed, e.g., once the treatment electrode is appropriately positioned. FIG. 12B provides a cross-sectional view of a treatment device 1200 with no optional stylet present, allowing for a clear fluid path/lumen 1230 through the treatment device, including the treatment tip region and including a distal/tip electrode) 380 via the lumen opening 1220 to the fluid path/lumen 1230. In the embodiments of FIGS. 12A and 12B, and applicable to and combinable with other examples herein, treatment electrode (distal/tip electrode) 380 may be, for example, a nitinol or stainless-steel electrode or any other suitable electrically conductive material. Stylet 1240 may be, for example, any suitable material, such as nitinol. The needle electrode tip can be any appropriate size, for example, between about 18 G and about 23 G. In particular examples, a higher gauge needle, e.g., a 23 G needle or higher, may be appropriate.

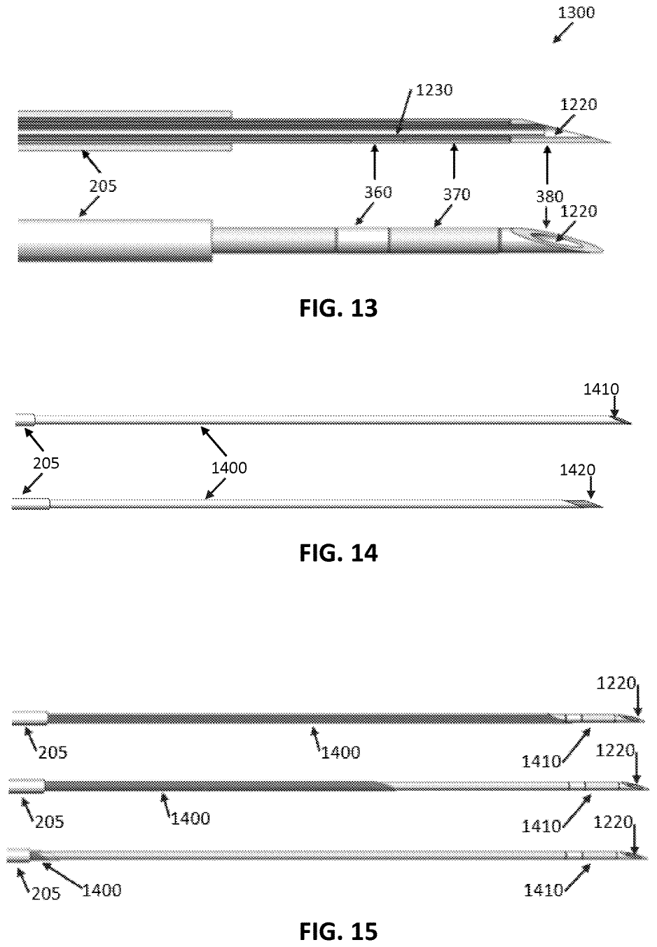

FIG. 13 provides an example of an apparatus capable of providing electric treatment to a target site and, further, infusion of, e.g., a fluid such as saline or a therapeutic agent to the target site. In certain implementations, the fluid path may be narrow, and thus the apparatus may be suitable for, e.g., infusion, and its suitability for aspiration may be limited. However, in other implementations, a fluid path may be sufficiently wide and, thus, the design of an apparatus such as that exemplified in FIG. 13 may be suitable for both infusion and aspiration (e.g., collection of material).

FIG. 13 illustrates exemplary treatment device 1300 comprising a proximal electrode 360, a distal electrode 380, including lumen opening 1220. Similar to other disclosed embodiments, an insulative or conductive spacer 370 may be positioned between the proximal and distal electrodes. The treatment device of FIG. 13 is capable of providing bipolar electrical treatment. Treatment device 1300 comprises a fluid path/lumen 1230. As embodied in FIG. 13, a distal electrode 380 can comprise a bevel tip design comprising the channel (lumen) passing through the electrode and establishing fluid path/lumen 1230. A distal electrode 380 and, likewise, the proximal electrode 360, can comprise any number of lumens. One or more lumen openings can serve as a port, e.g., an infusion port, aspiration port, or both. As described elsewhere herein, when a plurality of lumens are present, they may share one or more characteristics, such as, e.g., a central axis, size (inner or outer diameter or both). In various examples, any two or more present lumens may differ in one or more characteristics compared to any one or more other lumen(s) present.

FIG. 14 illustrates an embodiment of an apparatus comprising at least one relatively large central lumen to enable faster aspiration before and during the procedure. Such an embodiment may also or alternatively facilitate the collection of, e.g., thicker fluid/tissue than may otherwise be feasible through smaller lumens. Device of FIG. 14 may be guided through the scope using an introducer shaft 205.

The insertion/aspiration tube (which may have a needle-type tip) 1400 may comprise mechanism(s), for example, in a handle for facilitating the application of an expelling pressure or, also or alternatively, a vacuum pressure, as applicable to the selective delivery or collection of material(s). Insertion/aspiration tube 1400 comprises at least one lumen (not shown) that may be similar to a fluid path/lumen 1230 of FIG. 13 but may have a larger diameter or circumference. Such lumen may also facilitate the delivery of one or more treatment electrodes to a target site. Lumen 1410 may facilitate the use of electrode treatment devices of a size 20 G or lower, for example, 19 G or lower. As with all disclosure herein, description(s) of features here and elsewhere can be combined in various ways to establish apparatus(es) having a diversity of combinations of features. For example, an insertion/aspiration tube 1400 may comprise, one or a plurality of lumens, such lumen(s) having a plurality of possible configurations (concentric, non-concentric, uniform and non-uniform inner and outer diameters, and the like). Insertion/aspiration tube 1400 may be configured to facilitate use of a stylet 1420 (FIG. 14, bottom) or a plurality of stylet(s) to block one or more lumens 1410 of an insertion/aspiration tube during placement.

Moving to FIG. 15, exemplary application or use of an insertion/aspiration tube/needle 1400 is illustrated. Aspiration/insertion tube/needle 1400 may comprise any suitable material, including without limitation, stainless steel, nitinol, hard plastic (e.g., PEEK), and similar materials known in the art. Insertion/aspiration needle 1400 may extend from introducer shaft 205. FIG. 15 (top) demonstrates that once the distal tip of aspiration/insertion needle is positioned at a target treatment site, any present stylet 1420 (see FIG. 14; not shown in FIG. 15) may be removed, and a mono- or bipolar electrode device comprising an electrode treatment tip region 1410 may be inserted. One or more electrodes may be present on an electrode treatment tip region 1410. For example, in monopolar implementations, a single distal electrode may be used, while in bipolar configurations, for example, as shown in FIG. 15, a bipolar catheter-based device may comprise a distal and a proximal electrodes separated by a spacer. In the provided examples of FIGS. 14 and 15, collection of material, delivery of material, or both collection and delivery of material may be accomplished through at least one lumen therethrough to facilitate both the delivery of electrical energy and the delivery or collection of material(s) as described herein. During treatment, the insertion/aspiration tube may be pulled back, for example, into a handle of the device to partially or fully expose the tip region with treatment electrode(s), as well as create adequate separation between the treatment electrode(s) and the insertion/aspiration tube, which is especially applicable when applying high voltage pulses. FIG. 15 demonstrates in 3 views (top, middle and bottom) various stages of withdrawal/pulling back of the insertion/aspiration tube to expose the tip region of the device. FIG. 15 (top) illustrates the aspiration/insertion needle 1400 positioned in an extended configuration relative to introducer shaft 205, with the electrode treatment tip region 1410 partially extending from the aspiration insertion needle. FIG. 15 (middle) illustrates the aspiration/insertion needle 1400 in an intermediate or further pulled back position relative to introducer shaft 205. Likewise, FIG. 15 (bottom) illustrates the aspiration/insertion needle 1400 almost fully pulled back relative to introducer shaft 205.

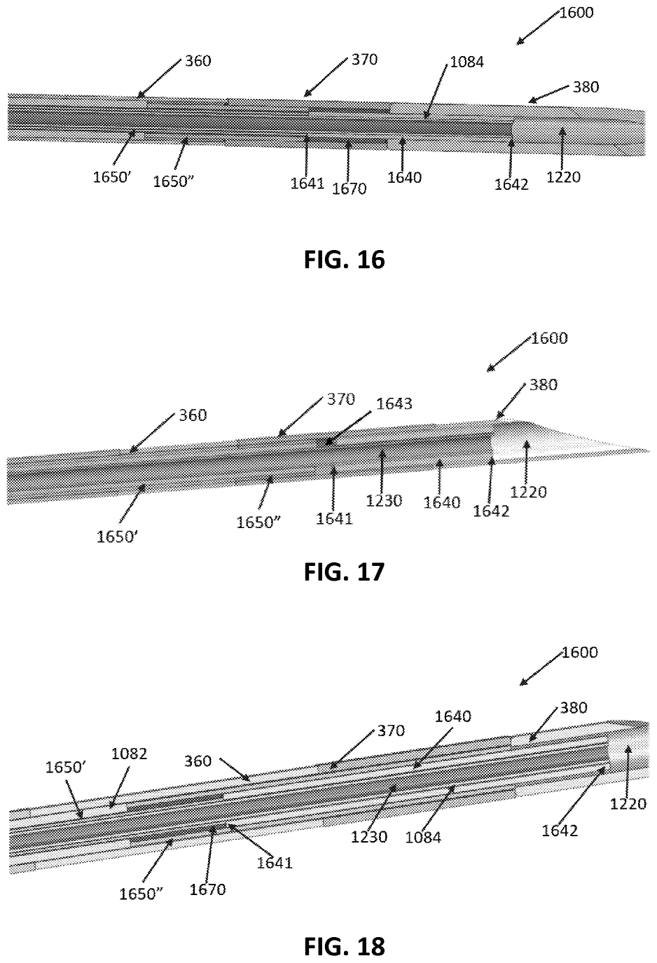

In some implementations, especially those requiring low profile/small sizes and bipolar configuration using high voltage fast electric pulses, conventional devices face significant challenges such as undesirable flexing/bending or other structural instability including, e.g., breakage due to lack of strength, as well as potential issues related to proper isolation of the electrodes having a voltage (potential) differential between them (e.g., having opposite polarities), such as electric shock, arcing, burns and resulting damage to the device and a patient. Devices and methods according to further examples of the present disclosure address these problems and provide technological solutions discussed below. For example, with a need for smaller devices, there is a need to reduce the outside diameter of the elongate body of the treatment tool, including in the tip region. As the outside diameter and/or wall thickness is reduced, the elongate shaft of the device losses stiffness and strength. While catheter-based devices require certain flexibility along their elongated body, it is desirable to maintain stability of the treatment tip region with electrode(s), for example, structural stability and strength between the proximal and distal electrodes, prevent any significant bending or flexing of the treatment tip region between the electrodes, including during travel to, and positioning at or within, a target treatment site. This may be particularly relevant in apparatuses designed to be very small, e.g., having electrode treatment tips of 23 G, 24 G, or smaller. To facilitate such structural stability and strength in the treatment tip region while still preserving necessary distance and separation between various conductive parts as well as overall flexibility of the device, one or more support members may be implemented as discussed by example in reference to FIGS. 16-18. In some examples, specific longitudinal/axial spacing between component(s), radial spacing between component(s), or both are selected and may be required to facilitate achieving a low profile (e.g., 23 G, 24 G, or smaller) treatment tip region, wherein the treatment tip region may still provide electrical treatment, e.g., high voltage bipolar electrical treatment such as that described herein, to a target site while meeting practical and/or required safety standards, such as avoiding or at least reducing the risk of arcing that could otherwise occur due to the existence of voltage differentials.

In some examples, one or more support members may be positioned at least partially between two electrodes, e.g., proximal and distal electrodes of a treatment tip region that extends distally from an elongate shaft of treatment tools of the present disclosure. In some examples, one or more support members may be concentrically arranged (e.g., sharing an axis) relative to two electrodes, e.g., first (proximal) and second (distal) electrodes.

In one example, a support member may not extend distally beyond the distal end of a distal electrode and may not extend proximally beyond the proximal end of a proximal electrode. If more than two electrodes are present, in some examples, a support member may not extend distally beyond the distal end of the most distal electrode and may not extend proximally beyond the proximal end of the most proximal electrode.

FIG. 16 illustrates one example of a structurally supported bipolar electrode treatment tip region of a device herein. Electrode treatment tip region 1600 comprises a proximal electrode 360, a distal electrode 380, and an electrode spacer 370 disposed therebetween. Each of, e.g., the distal and proximal electrodes, as well as the electrode spacer, may comprise any one or more features thereof described elsewhere in this disclosure; such as, e.g., distal electrode 380 may comprise a piercing tip having, e.g., a trocar design, a beveled design, a hybrid design, etc. Further, for example, electrode spacer 370 may be conductive or may be insulating. Note that one or more components present in a treatment tip region 1600 may be conductively/electrically associated with a proximal electrode 360 and a distal electrode 380. For example, hypotube or rod 1084 forming fluid path/lumen 1230 may comprise an electrically conductive materials, such as stainless steel or Nitinol, and connect to distal electrode 380 (similar to the hypotube or rod 1084 of FIG. 10B). Similarly, hypotube or rod 1082 (shown in FIG. 18) may comprise an electrically conductive material and connect with proximal electrode 360.

Electrode treatment tip region 1600 may comprise in some embodiments (optionally) an electrode treatment tip lumen opening 1220 facilitating access to a fluid path/lumen 1230 that can pass at least in part through an electrode treatment tip region, including through distal electrode 380. Such electrode treatment tip opening 1220 and fluid path/lumen 1230 may facilitate the delivery or aspiration of materials, such as the delivery of therapeutic agent(s) such as drug(s), the collection of cells or tissue, etc. as is described elsewhere herein, while the electrodes of the electrode treatment tip region are designed for the delivery of electric treatment to target tissue. As disclosed elsewhere, contemplated are embodiments wherein a plurality of lumens is present, either, e.g., concentrically aligned, non-concentrically aligned, sharing at least the same inner diameters, outer diameters, or both, differing in their inner or outer diameters or both, or combinations thereof.

In the embodiment of electrode treatment tip region 1600 illustrated in FIG. 16, the electrode treatment tip comprises support member 1640, having a support member proximal end 1641 and a support member distal end 1642. Reference to a proximal end 1641 would be the most proximally positioned portion of the support member while reference to a distal end 1642 would be the most distally positioned portion of the support member. Support member 1640 may provide enhanced structural support to the treatment tip region, including reducing the flexibility and increasing the strength of the electrode treatment tip region between the proximal and distal electrodes. Support member 1640 may be a single component (or a plurality of connected components forming a support structure and providing relative rigidity to the tip region), such as, e.g., a band, tube, or shaft-like element, as provided by FIG. 16. In some examples, support member 1640 may be formed together with the distal electrode 380 as one piece, or soldered together. In some examples, support member 1640 can be a separate and independent component from the distal electrode. According to various embodiments, apparatuses/devices disclosed herein are configured for accessing particularly small anatomical spaces. In some embodiments, the outer diameter(s) or circumference(s) of device(s) is very limited. In various examples, support member(s) are designed as bands/tubes/shafts configured to facilitate a desirable torsional strength, desirable limitation of flexibility, or both, while further allowing for the treatment tip region to be suitably small for accessing very tight anatomical space(s) and/or while providing necessary spacing and isolation of the conductive parts of different polarity.

Support member 1640 may be positioned within the walls of the electrode treatment tip region 1600, such that it is not externally visible, and it may be concentrically arranged relative to the first and second electrodes. Support member 1640 may be present only within a portion of an electrode treatment tip and, also or alternatively, may be absent from any other part of a catheter body of which the electrode treatment tip region is a component.

Support member 1640 may be made of any suitable material, such as, e.g., stainless steel, nitinol, or any other suitable and sufficiently strong, rigid material capable of providing structural strength and support to the electrode treatment tip region. Support member 1640 may comprise a conducting material or a non-conducting material. In still other examples, support member 1640 may comprise an insulating material. In some examples, support member 1640 can comprise two or more materials, such as, e.g., a combination of conducting materials, a combination of non-conducting materials, a combination of insulating materials, or any further combinations thereof.

Support member 1640, as exemplified in FIG. 16 may partially bridge the proximal electrode and the distal electrode, spanning less than the entire distance between them. For example, support member 1640 may span less than 100% of the distance between a proximal electrode and a distal electrode, such as, e.g., less than 95%, 90%, 85%, 80%, 75%, 70%, 65%, 60%, 55%, 50%, 45%, 40%, 35%, 30%, 25%, or less than 20% of the distance between proximal and distal electrodes.

FIG. 16 provides an example of a partially supported tip region, including a support member 1640 extending (starting at its distal end 1642) from the distal electrode proximally toward, but not reaching, the distal end of a proximal electrode, wherein the proximal end 1641 of the support member is positioned between the proximal and distal electrodes. In an alternative embodiment (not shown), support member 1640 may extend (starting at its proximal end 1641) from the proximal electrode distally toward, but not reaching, the proximal end of a distal electrode, wherein the distal end 1642 of the support member is positioned between the proximal and distal electrodes.

For providing strength and alignment, in some embodiments, as those shown in FIGS. 16 and 17, a support member 1640 can axially (along a longitudinal axis of the tip region) physically overlap either a portion of a proximal electrode 360 or a portion of a distal electrode 380 by any suitable amount, such as by 0.75×, 1×, 1.25×, 1.5×, 2×, 2.25×, or even 2.5× or more, for example by about 2× the diameter of the support member. The actual amount of overlap in mm, cm or inches may vary depending on the size of the device, relevant procedure, a target issue, etc. In one non-limiting example, a support member 1640 may axially overlap a portion of a proximal electrode 360 or a portion of a distal electrode 380 by at least 0.3 mm, at least 0.4 mm, at least 0.5 mm, at least 0.6 mm, at least 0.7 mm, at least 0.8 mm, at least 0.9 mm, at least 1 mm, at least 1.1 mm, at least 1.2 mm, at least 1.3 mm, at least 1.4 mm, at least 1.5 mm, at least 5 mm, at least 10 mm, etc. However, since the first (proximal) and second (distal) electrodes may be configured to have a voltage differential (for example, including but not limited to having opposite polarity), the support member, while physically overlapping with (and being electrically connected to) one electrode, such as the distal electrode 380 as shown in FIG. 16, would need to have electrical separation from the proximal electrode 360. As will be understood by those skilled in the art, if, alternatively, the support member physically overlaps with (and electrically connected) to the first or proximal electrode, then it would have to be electrically separated from the distal electrode having a voltage (potential) differential relative to the proximal electrode (e.g., opposite polarity). The outer diameter of the support member may vary depending on a particular implementation. For example, in some embodiments, a support member 1640 may have an outer diameter between about 0.15 mm and about 2.5 mm, and, as stated above, for providing a strength, support and/or alignment, it may overlap with a proximal electrode 360 or a distal electrode 380, for example, by approximately between about 0.15 mm and about 3.5 mm, such as approximately between about 1× and about 2× (or more) the support member outer diameter. The electrical separation of the support member from any electrode to which it is not conductively/electrically connected (which is proximal electrode 360 in the example of FIG. 16) may be based, at least in part, on a dielectric characteristic (e.g., strength) of any present insulative and/or adhering/adhesive material and the voltage differential between the electrodes, as discussed in more detail below.

Electrode treatment tip region 1600 may further comprise insulative material 1650′ and 1650″. Insulative material may be present in a plurality of layers, bands, tubes, or other such structures to insulate one electrode from another. In the provided embodiment, a first, e.g., “inner” layer, e.g., inner tube, of insulative material 1650′ extends along a length of a catheter shaft (of which the electrode treatment tip region represents the distal end). The inner layer of insulative material in the illustrated embodiment extends such that its distal end meets, e.g., abuts, the proximal end 1641 of support member 1640. In some examples, as shown in FIG. 16, a second layer (e.g., tube) of insulative material 1650″ is positioned externally to the inner layer of insulation (but not on the exterior of the electrode treatment tip). In this example, the second layer of insulative material 1650″ at least partially overlaps the inner layer of insulative material 1650′ but is shorter in length than the layer of inner insulative material 1650′. In some examples, the distal end of the second layer of insulative material 1650″ may end at the same location along the electrode treatment tip region 1600 as the inner layer of insulative material 1650′. While it may end at the same location along the electrode treatment tip region 1600 as the inner layer of insulative material 1650′, as this second layer 1650″ is positioned above or external to the inner layer of insulative material 1650′, it may not entirely abut the proximal end 1641 of the support member 1640. In some examples, the proximal end of the second layer of insulative material ends within the electrode treatment tip region 1600, not extending proximally beyond the electrode treatment tip region up the catheter shaft, toward, e.g., an apparatus handle.

FIG. 16 further illustrates that electrode treatment tip region 1600 can comprise, e.g., connecting or adhering material 1670 (e.g., glue/adhesive) in one or more locations. Connecting or adhering material/adhesive 1670 can be used to secure one or more components of the treatment tip region to other(s). As seen in FIG. 16, proximal electrode 360 is electrically separated from the support member 1640 in an axial direction (along a length of the tip region). In this example, electrical separation (gap) of the support member from the proximal electrode 360 in axial direction is based, at least partially, on dielectric properties of any one or all of the insulative material 1650′, 1650″, and adhesive 1670, as well as voltage differential between the electrodes. In certain examples, proximal electrode 360 may be additionally separated from support member 1640 in a radial direction. Such a radial separation is described in reference to FIG. 18.

FIG. 17 illustrates another example of a structurally supported bipolar electrode treatment tip region of a device of the present disclosure. Similar to FIG. 16, the tip region 1600 of this example comprises a proximal electrode 360, a distal electrode 380, and a spacer 370 disposed therebetween. Each of, e.g., the distal and proximal electrodes, as well as the electrode spacer, may comprise any one or more features thereof described elsewhere in this disclosure; such as, e.g., distal electrode 380 may comprise a piercing tip having, e.g., a trocar design, a beveled design, a hybrid design, etc. Further, for example, electrode spacer 370 may be conductive or may be insulating.

Electrode treatment tip region 1600 may comprise an electrode treatment tip lumen opening 1220, opening to a fluid path/lumen 1230 (as seen in FIG. 12B). The fluid path/lumen 1230 may extend through the length of the electrode treatment tip region (also referred to herein as a tip region), including through the distal electrode 380. Fluid path/lumen 1230 and the lumen opening 1220 facilitate the delivery or aspiration of materials, such as the delivery of fluid(s) or therapeutic agent(s) such as drug(s), the collection of cells or tissue, etc. as is described elsewhere herein, while the electrodes of the tip region are configured for the delivery of electric treatment to target tissue.

In the embodiment of electrode treatment tip region 1600 illustrated in FIG. 17, the electrode treatment tip region comprises support member 1640, having a support member proximal end 1641 and a support member distal end 1642. Support member 1640 may provide enhanced structural support to the electrode treatment tip region, reducing the flexibility and increasing the strength of the electrode treatment tip region between the proximal and distal electrodes.

As the support member 1640 of FIG. 16, support member 1640 of FIG. 17 may be made of any suitable material, such as, e.g., stainless steel, nitinol, or any other suitable and sufficiently strong, rigid material capable of providing structural strength and support to the electrode treatment tip.

Like the support member illustrated in FIG. 16, support member 1640 in the example of FIG. 17 may partially bridge the proximal electrode 360 and the distal electrode 380, spanning less than the entire distance between them. For example, support member 1640 may span less than 100% of the distance between a proximal and a distal electrode, such as, e.g., less than 95%, 90%, 85%, 80%, 75%, 70%, 65%, 60%, 55%, 50%, 45%, 40%, 35%, 30%, 25%, or less than 20% of the distance between a proximal and distal electrode.

FIG. 17 shows another example of a partially supported tip region, including a support member 1640 extending (starting at its distal end 1642) from the distal electrode proximally toward, but not reaching, the distal end of a proximal electrode, wherein the proximal end 1641 of the support member is positioned between the proximal and distal electrodes.

For providing strength and alignment, in some embodiments a support member 1640 can axially (along a longitudinal axis of the tip region) physically overlap either a portion of a proximal electrode 360 or a portion of a distal electrode 380 by any suitable amount, such as by 0.75×, 1×, 1.25×, 1.5×, 2×, 2.25×, or even 2.5× or more, for example by about 2× the diameter of the support member. For example, a support member 1640 may axially overlap a portion of a proximal electrode 360 or a portion of a distal electrode 380 by at least 0.3 mm, at least 0.4 mm, at least 0.5 mm, at least 0.6 mm, at least 0.7 mm, at least 0.8 mm, at least 0.9 mm, at least 1 mm, at least 1.1 mm, at least 1.2 mm, at least 1.3 mm, at least 1.4 mm, at least 1.5 mm, at least 5 mm, et least 10 mm, etc. Tip region of the apparatus of FIG. 17 may have a larger diameter than the example of FIG. 16. For example, this apparatus may be 18 G. In some implementations, a support member 1640 may have an outer diameter between about 0.3 mm and about 2 mm, and it may overlap with a proximal electrode 360 or a distal electrode 380 for example, approximately between about 1× and about 2.5× (or more) the support member outer diameter.

Electrode treatment tip region 1600 may further comprise insulative material 1650′ and 1650″. Insulative material may be present in a plurality of layers, bands, tubes, or other such structures to insulate one electrode from another. In the provided embodiment, the position, sizing and construction of the insulative layers may be as provided elsewhere in the present disclosure. Area 1643 identified in FIG. 17 may comprise, e.g., an adhesive material 1670 or an insulative material, e.g., 1650′ or 1651″. As explained in reference to FIG. 16, proximal electrode 360 is electrically separated from the support member 1640 in an axial direction (along a length of the tip region In this example, electrical separation (gap) of the support member from the proximal electrode 360 in axial direction is based, at least partially, on dielectric properties of any one or all of the insulative material 1650′, 1650″, and adhesive 1670, as well as voltage differential between the electrodes.

FIG. 18 illustrates an alternative configuration of a tip region 1600 comprising a support member 1640 that provides both distal and proximal support in the tip region. The example of FIG. 18 provides more support to the tip region of the apparatuses of the present disclosure than the examples of FIG. 16 and FIG. 17. It may, in particular scenarios, be advantageous to use one design in place of another.

While FIGS. 16 and 17 demonstrate examples of a partially braced (supported) design of the tip region of the treatment tool of the present disclosure, FIG. 18 illustrates an example of a structurally supported multi-electrode, e.g., bipolar electrode treatment tip region 1600 with a fully braced design. In the embodiment of FIG. 18, electrode treatment tip region 1600 comprises a proximal electrode 360, a distal electrode 380, and an electrode spacer 370 disposed between them. As provided elsewhere, each the distal and proximal electrodes, as well as the electrode spacer, may comprise any one or more features thereof described elsewhere in this disclosure.

As provided elsewhere herein, a treatment tool may comprise a hypotube or rod 1082 configured to assist in connecting the proximal electrode to the source of electric energy. The rod 1082 may comprise an electrically conductive material, for example, stainless steel or Nitinol, and may electrically connect to and share voltage with the proximal electrode 360. Also, as provided elsewhere herein, support member(s) can be conductive or alternatively non-conductive. In the example of FIG. 18, support member 1640 may be conductive. A voltage differential exists between proximal electrode 360 and distal electrode 380; for example, proximal electrode 360 and distal electrode 380 may comprise opposing polarities. In this example, support member 1640 is in contact with distal electrode 380 and comprises the voltage of the distal electrode 380, establishing a voltage differential between it and proximal electrode 360.

As in FIG. 16, in this example a fluid path/lumen 1230 is formed by a conductive hypotube or rod 1084 that is in electrical contact with distal electrode 380 via support member 1640. As previously stated, conductive hypotube or rod 1084 forms a lumen (e.g., fluid path) through to the opening in distal electrode 380. Accordingly, in the provided illustrated embodiment, the hypotube/rod 1084 comprises the same voltage as distal electrode 380. In other implementations, instead of through the conductive hypotube 1084, distal electrode 380 may be electrically connected to the electrical source (e.g., pulse generator) through a wiring that may run, for example, from the distal electrode along a length of the tip region and into a handle of the apparatus. Similarly, in alternative embodiments, support member 1640 may comprise a non-conductive material. In such an embodiment, to establish an electrical path from an electrical source to the distal electrode 380, a conductive hypotube 1084 may be electrically connected to the distal electrode, or wiring may be used to make such a connection, just to name few examples.

As previously stated, electrode treatment tip region 1600 may comprise an electrode treatment tip lumen opening 1220 (not shown) opening to a fluid path/lumen 1230. Fluid path/lumen 1230 may extend through the length of the electrode treatment tip region, including through distal electrode 380 and proximal electrode 360.

In the embodiment of electrode treatment tip region 1600 illustrated in FIG. 18, the electrode treatment tip region comprises support member 1640, having a support member proximal end 1641 and a support member distal end 1642. The embodiment of support member 1640 of FIG. 18 may provide enhanced structural support to the electrode treatment tip region by physically overlapping both distal and proximal electrode, reducing the flexibility and increasing the strength of the electrode treatment tip region between the proximal and distal electrodes.

Support member 1640 may be positioned within the walls of the electrode treatment tip region 1600, such that it is not externally visible. Support member 1640 may be present only within a portion of an electrode treatment tip region and, also or alternatively, may be absent from any other part of a catheter body of which the electrode treatment tip region is a component. For example, Support member 1640 may be made of any suitable material or combination of suitable materials as previously described. In some examples, a support member 1640 may comprise a wall thickness of, e.g., between about 0.015 mm and 0.35 mm.

Unlike the example of support member 1640 of FIGS. 16 and 17, as explained above, support member 1640 of the treatment tip region 1600 illustrated in FIG. 18 completely bridges, e.g., provides a continuous connection between, the proximal electrode 360 and the distal electrode 380, spanning the entire distance between them. For example, as shown in FIG. 18, a distal end 1642 of the support member may be concentrically positioned somewhere along a length of the distal electrode and the proximal end 1641 of the support member may be concentrically positioned somewhere along a length of the proximal electrode, thereby concentrically overlapping with both the distal and proximal electrodes along a longitudinal axis of the tip region.

Support member or brace 1640 can overlap a portion of a proximal electrode 360 and a portion of a distal electrode 380 by any suitable amount, such as by 0.75×, 1×, 1.25×, 1.5×, 2×, 2.25×, or even 2.5× or more of the outer diameter of support member 1640, for example by about 2× the outer diameter of the support member. The amount of overlap between a support member 1640 and a proximal electrode 360 may be the same or different from the amount of overlap with a distal electrode 380.

In an example of FIG. 18 where a support member 1640 longitudinally (axially) overlaps two electrodes to provide strength, structural stability and, potentially, improved alignment, at least on one side (shown by example on the proximal electrode side) this physical overlap is electrically isolated. If a voltage differential exists between a proximal electrode 360 and a distal electrode 380 while the support member is electrically connected to the distal electrode, the support member needs insulation from the proximal electrode (and from components sharing a voltage with such an electrode, such as for example hypotube 1082) to avoid arcing and other negative consequences, as explained above. In some examples, apparatuses comprising support member(s) as described herein, for example, when bipolar electrode configurations are present in the apparatus, a support member that comprises the polarity of a first electrode may be appropriately separated longitudinally/axially, radially, or both longitudinally/axially and radially from any other electrode (or associated conductive component) with which there exists a voltage differential to eliminate or at least to reduce the risk of arcing. The amount of separation (e.g., the size of the “gaps”) between a support member and an electrode longitudinally/axially, radially, or both is/are at least in part dictated by the configuration of components present in the apparatus and the material(s) of such component(s), such as the dielectric strength of adhesive(s) used, characteristic(s) of insulating materials, relationship between electrodes and other electrically conducting components, voltage differential(s) between electrodes, and the like, or combinations thereof. In some examples, a support member may be radially separated (e.g., at least along a portion of a diameter/thickness of the tip region) from any electrically conductive component comprising a voltage differential (such as an opposing polarity) by a distance, by insulating materials, or both. Such radial separation may be based, at least partially, on the size of the components and overall device, including, e.g., wall thickness(es).

In various examples, as stated elsewhere, a support member may be formed together with a distal electrode (e.g., whereby the component is manufactured as a single piece/part). In some examples, a support member can be, e.g., functionally connected to an electrode, e.g., by soldering or other suitable attachment mechanism. In some examples, a support member may comprise a conductive material and thus comprise a voltage (and in some cases a polarity) associated with one or more electrodes.

Electrode treatment tip region 1600 may further comprise insulative material 1650′ and 1650″. Insulative material may be present in a plurality of layers, bands, or other such structures to insulate one electrode from another. In the example of FIG. 18, inner insulation is composed of two overlapping/nesting insulating lumens 1650′ and 1650″ creating a stepped transition. Thickness of a layer of insulative material 1650′, 1650″, or both may be determined based upon characteristic(s) of the material, the electrical environment (e.g., voltage differential(s)) between component(s) it is serving to insulate, the configuration of component(s) and characteristic(s) thereof, etc.

In the example of FIG. 18, overlapping/nesting insulative layers (1650′ and 1650″) create a stepped transition that at least in part facilitates a treatment tip region of an apparatus having a reduced outer diameter (e.g., 23 G or 24 G or smaller) while still enabling bipolar electrical treatment, and optionally infusion, aspiration or both, while reducing or eliminating the risk of arcing. In some embodiments, the presence of overlapping layers of insulation establishes a radial distance that contributes, at least in part, to maintaining sufficient insulation (and/or, e.g., a sufficient insulative distance) between components comprising opposing polarities, e.g., bipolar electrodes. In various examples, the radial thickness created by the overlapping/nesting insulative layers 1650′ and 1650″ (and/or the adhesive layer, when present) illustrated in FIG. 18 is at least in part dictated by physical requirement(s), e.g., wall thickness and sizes of component(s) present in the treatment tip region, while the length of the overlap between the insulative layers 1650′ and 1650″ is at least in part dictated by the dielectric properties of the insulative layers and voltage differentials between components of different polarities, e.g., a proximal electrode (and conductive component(s) to which it is conductively associated) and a distal electrode (and conductive component(s) to which it is conductively associated). One of the advantages of the design and configuration of treatment tip regions of apparatuses described herein, is that they can access very small anatomical spaces; that is, the outer diameter of the treatment tip region is very small, such as 23 G or 24 G or smaller in size while still providing strength and structural stability to the apparatus. Without a support member disclosed herein, the treatment tip region may lack sufficient strength and rigidity, and may be subject to breaking, as well as creating user challenges when navigating the tip to a target tissue. A stepped configuration of insulative layers may aid in allowing for a very small outer diameter of the device, small inner infusion/aspiration lumen, while facilitating the presence of a support member that increases structural stability and strength of a treatment tip region comprising a plurality of electrodes, e.g., bipolar electrodes.

In the embodiment illustrated in FIG. 18, the proximal end of the second layer of insulative material ends within the electrode treatment tip 1600, not extending proximally beyond the electrode treatment tip region up the catheter shaft, toward, e.g., an apparatus handle. The distal end of the second layer of insulative material ends at the same position along the length of the electrode treatment tip region as the distal end of electrode spacer 370.

FIG. 18 further illustrates that electrode treatment tip 1600 can comprise, e.g., connecting or adhesive material 1670. Connecting or adhering (adhesive) material 1670 can be used to secure one or more components of the tip region to other(s). An adhesive material may be, for example, a glue. In certain aspects, two or more component(s) may be associated with one another by adherence such as by soldering, a laser weld, press fit, use of heat/cold to create fit, etc. The location(s) of adhesive material 1670 shown in FIGS. 16-18 are examples only. Adhesive material 1670 may also or alternatively be located in other location(s) and, depending on the location, the components being associated with one another and the relationship of conductivity between them, the form of adherence may be established accordingly. Dielectric strength of adhesive material 1670, as well as characteristic(s) of insulative material 1650′, 1650″, or both, may in various examples at least in part establish the suitable longitudinal/axial separation distances, radial separation distances, or both between components having voltage differential.

According to some implementations, the apparatuses/treatment tools of the present disclosure are configured for use in very tight anatomical spaces. Accordingly, the significantly limited space within components required creative changes to the design to accommodate the requirements of such reduce sizes of the components. In addition, the bipolar configuration of closely spaced distal and proximal electrodes having a voltage differential between them, especially when configured for delivery of high voltage very fast electric pulses, requires that the electrodes and their associated wiring be sufficiently insulated from one another in/from multiple directions. Given the limited space, the size of the electrodes and the thickness of the insulation between them is relevant. For example, insulation must be sufficiently robust to prevent arcing yet not so thick or robust that it unsatisfactorily increases the diameter of the electrode treatment tip region. In various examples, the presence of insulation, e.g., its amount or thickness, may be a function of the dielectric strength of the insulator and, e.g., the voltage differential between components it is insulating. Therefore, it will vary depending on particular implementation. Just as non-limiting example, in some implementations such insulation may be less than 0.01 inches thick, such as, e.g., less than 0.009, 0.008, 0.007, 0.006, or less than 0.005 inches thick. For example, insulation may be between 0.0005 and about 0.0015 inches thick. In various examples, depending on the voltage of the device, an insulation thickness may be increased or decreased as appropriate. Moreover, desirability of having additional lumen(s) for aspiration and/or infusion create additional spacing considerations that further affect overall configuration and design. The novel configurations described herein address various above-mentioned challenges and considerations. For example, with respect to some embodiments with support member, Applicant discovered that to maintain advantages and be functional, the support member must be sized, configured and positioned with at least approximately 1× diameter offset for metal-to-metal overlap and may have a minimum gap based on voltage and adhesive/insulative material dielectric strength to electrically separate the distal and proximal conductive components. As stated elsewhere, a required longitudinal/axial distance between a support member and any electrode to which it is not conductively (electrically) connected, e.g., a proximal electrode, may be based, at least in part, on the dielectric strength of any present adhering or insulative material and the voltage differential between electrodes. In some implementations, a required longitudinal distance between the proximal end of a support member and a proximal end of any electrode to which it is not conductively/electrically connected (e.g., the proximal electrode) may be based, at least in part, on the dielectric strength of any present adhering material and/or insulative material/layers and the volage differential between electrodes.

With respect to the aspiration/infusion lumen, such a lumen may be between about 5 mm and about 3000 mm or longer, such as, e.g., between about 50 mm and about 300 mm long or, e.g., between about 1000 mm and 3000 mm or between about 1500 mm and about 2500 mm long. In some non-limiting examples, the length may depend on a procedure type, target tissue and other considerations. In some non-limiting examples, an aspiration/infusion lumen can comprise an inner diameter of between about 0.05 mm and 0.7 mm, such as between about 0.1 mm and about 0.2 mm or between about 0.3 mm and about 0.6 mm.

In some additional examples, a support member 1640 of a treatment tip region of devices described herein may extend from a distal electrode proximally such that it overlaps with up to, e.g., 80%, 85%, 90%, 95%, or 100% of the length of a proximal electrode 360. In some implementations, a support member 1640 comprises a conductive material and/or electrically coupled to a distal electrode, and therefore, requires electrical separation from a proximal electrode. For example, such a separation can be, e.g., at least about 0.01 mm, 0.015 mm, 0.02 mm, 0.025 mm or, more. In examples, such a separation may be at least about 0.05 mm, 0.1 mm, 0.2 mm, 0.3 mm, 0.4 mm, 0.5 mm, 0.6 mm, 0.7 mm, 0.8 mm, 0.9 mm, or, about 1 mm (or more). In some implementations, a treatment tip region 1600 of a device herein can comprise a separation or gap between a support member 1640, e.g., a proximal end 1641 of a support member 1640, and a component with which there is a voltage differential, e.g., a component having the polarity of the proximal electrode 360, e.g., the hypotube 1082, such as the distal end of hypotube 1082, suitable for preventing arcing or other damage to the devices and patients. In certain implementations, a treatment tip region 1600 of a device can comprise, e.g., a separation or gap between a support member 1640, e.g., a proximal end 1641 of a support member 1640, and a proximal electrode 360 of any suitable amount, such as by approximately 0.5×, 1×, 1.25×, 1.5×, 2×, 2.25×, or even 2.5× or more of the outer diameter of support member 1640.

In alternative examples, wherein, for example, a support member 1640 is electrically coupled to a proximal electrode, similar concepts may apply, wherein, e.g., a suitable insulative space or gap may exist between a distal end 1642 of a support member 1640 and, e.g., a distal electrode 380.

In some examples, a treatment tip region comprises an axial overlap of a proximal electrode 360 and a hypotube 1082. Such overlap of a proximal electrode 360 with the hypotube 1082 may be, for example, 0.5×, 1×, 1.25×, 1.5×, 1.75×, 2×, 2.25×, or 2.5× (or more) the outer diameter of the hypotube 1082. Furthermore, a support member 1640 in conjunction with surrounding members (supportive layers) of a treatment tip region 1600 must be sufficiently rigid/non-flexing to be at least substantially resistant to column load(s) (buckling) resulting from the puncture force of the treatment tip region against a target tissue (e.g., tissue wall).

According to a further aspect of the present disclosure, methods of treatment of a target tissue are provided. In some examples, the method may comprise inserting percutaneously a tip region of a treatment tool into a target tissue, the tip region comprising a first electrode and a second electrode; and applying a plurality of electrical pulses having a pulse duration of less than 1000 nanoseconds while reducing peak electric field for a given potential and/or increasing hoop stress on the target tissue to prevent or at least reduce arcing between the first electrode and the second electrode. The method (and, e.g., other method(s) of using apparatus(es) disclosed herein) may be performed under image guidance, for example ultrasound imaging, or robotic system imaging. The method may comprise navigating and tracking percutaneous insertion of the tip region of the treatment tool, treatment planning and confirmation. In some examples, reducing the peak electric field comprises using a conductive spacer between the first electrode and the second electrode or using electrodes with the rounded corners. In some examples, increasing the hoop stress comprises using a spacer between the first electrode and the second electrode and wherein at least a portion of a circumference or a diameter of the spacer is larger (either permanently or only after being placed within a target area) than a circumference or diameter of each of the first electrode and the second electrode.

As stated above, the methods of the present disclosure may be used to treat lesions, tumors, tissue disorders and other abnormalities in or within muscular organs, circulatory organs, respiratory organs, abdomen and digestive organs, urinary organs, immune system organs, nervous system organs, endocrine organs, reproductive organs, or skeletal organs.

Uncontradicted, such methods may comprise use of ultrasound, such as, in examples, endoluminal ultrasound or endobronchial ultrasound, or other imaging technologies known at the time of this disclosure or which may be developed after the disclosure of the technology herein, including, e.g., cone beam CT and fluoroscopy.

For example, in some implementations, a method of treating a thyroid module is provided. The method comprises inserting percutaneously a tip region of a treatment tool into a target thyroid nodule, the tip region comprising a first electrode and a second electrode; and applying through the first electrode and the second electrode a plurality of electrical pulses having a pulse duration of less than 1000 nanoseconds to thyroid nodule while reducing peak electric field for a given potential and/or increasing hoop stress on the thyroid nodule to prevent or at least reduce arcing between the first electrode and the second electrode. Thyroid nodule may be a benign thyroid nodule. The method may also be conducted under image guidance.

As stated above, various apparatuses of the present disclosure may facilitate multiple modalities, such as ablation of a target site and infusion of material, such as a therapeutic agent, to the target site; or facilitate both ablation of a target site and extraction/removal of material, such as cells/tissue (e.g., for analysis/characterization), from the target site; or facilitate the ablation of a target site and the selective infusion and extraction of material(s) from the target site. In certain implementations, such multi-modal capability of apparatuses herein may reduce the time it takes to accomplish intended operations and treatments, the risk associated with such operations, the level of user frustration or error associated with a need to insert, remove, or both insert and remove a plurality of devices typically associated with accomplishing such operations and treatments, or any combination thereof.

According to some implementations, methods provided herein comprise application of an electrical treatment, e.g., ablation, to a target tissue and infusion of a material to the target tissue site, e.g., infusion of a fluid such as saline, medication, a therapeutic agent such as a drug, just to new a few. In some examples, methods/procedures of the present disclosure comprise (1) application of an electrical treatment, e.g., ablation, to a target tissue, (2) sample collection, and (3) infusion of a material to the target tissue site. In certain aspects described herein are apparatuses for use in ultrasound-guided procedures. In certain examples, apparatuses are designed for ablating tissue, acquiring fluid/cells/tissue from a target treatment site, delivering material (e.g., a therapeutic agent) to a target treatment site, or a combination thereof.

According to some implementations, apparatuses herein are characterizable as combination catheter-based devices suitable for use in endoscopic ultrasound-guided (EUS-guided) methods of tissue/fluid acquisition and ablation.

Provided herein are method(s) of treating a benign or malignant target tissue(s), such as a pancreatic tumor, with microsecond or sub-microsecond pulsed electric field using a multi-modal apparatus of the present disclosure. The method(s) may comprise delivering a treatment tip region of the multi-modal apparatus to a region of the target tissue, e.g., pancreatic tumor, of a subject, wherein the multi-modal apparatus comprises a tip region having a first electrode, a second electrode distal to the first electrode, a spacer between the first and second electrodes, and at least one infusion lumen. The method may further comprise applying a positive pressure to the treatment tip region of the multi-modal apparatus to deliver a material through the tip region to the target tissue, e.g., pancreatic tumor; maintaining the tip region in position at the target tissue, e.g., pancreatic tumor; and applying the microsecond or sub-microsecond pulsed electric field to the target tissue, e.g., pancreatic tumor. As provided, the target tissue, e.g., pancreatic tumor, may be benign or cancerous.

Further provided here are methods of treating a target tissue with a multi-modal treatment tool, the method(s) comprising: positioning a treatment tip region of the multi-modal treatment tool relative to a target tissue of a subject, the treatment tip region comprising a treatment electrode and one or more infusion and/or aspiration channels; applying a positive pressure to the treatment tip region such that a material is infused through the treatment tip region at or within the target tissue; applying a negative pressure to the treatment tip region to maintain a contact between the treatment tip region and the target tissue and/or to collect a sample from the target tissue; and applying pulsed electric field(s) to the target tissue using the treatment electrode of the treatment tip region. The method(s) may comprise any of the steps of applying positive pressure, applying negative pressure or applying pulsed electric field while maintaining the multi-modal treatment tool at the target tissue. For example, the application of the pulsed electric field may be before, after, or during the application of the negative pressure. Further, in some implementations, the infusion may be applied before or after the application of the pulsed electric field(s). The pulsed electric field(s) may be sub-microsecond (e.g., nanosecond) pulsed electric field(s). For example, collecting the sample may comprise performing a biopsy of the target tissue.

Any of the methods described herein may be adjusted for treatment of a variety of anatomical structures and tissues, including without limitation, muscular tissue, circulatory tissue, respiratory tissue, digestive tissue, nervous system tissue, reproductive organs tissue, cardiac tissue, just to new a few, and the methods may include any combination of steps of ablation, infusion and/or aspiration. Also, the multi-modal treatment tools used in such method may include any of the features described in reference to various examples and embodiments of the present disclosure.

Examples of methods, e.g., methods targeting a particular tissue or performed using particular guidance technology, are provided here.

According to some examples, apparatuses of the present disclosure may be used in an EUS-guided procedure for sampling and ablating a target location, such as, e.g., a pancreatic mass, e.g., a benign or malignant pancreatic mass, pancreatic cyst, pancreatic neuroendocrine tumor (pNET), or other GI lesion, or, e.g., other targets described elsewhere herein. In some examples, the apparatus is a single, multi-function needle device capable of tissue/fluid acquisition, suction/aspiration, biopsy and pulse filed ablation, including nanosecond (ns) pulsed field ablation. The apparatus may be a bipolar needle compatible with EUS systems that further comprises fine needle aspiration (FNA), fine needle biopsy (FNB), or both FNA and FNB tissue/fluid acquisition, and nsPFA ablation capabilities. Optionally, the device may further comprise the ability to dispense material. In examples, the needle of such an apparatus may enable both diagnostic sampling and therapeutic ablation during a single procedure, without requiring device exchange.

According to additional aspect of the present disclosure, disclosed herein are methods of performing an EUS-guided procedure for sampling and ablating a target tissue. An exemplary method may comprise an optional step of preparing and positioning the EUS system. The method may also comprise an optional step to appropriately position the patient (e.g., for accessing the left lateral decubitus for procedures directed to treatment of a pancreatic cyst, pNET, or other GI lesion). The patient may be under sedation or anesthesia.

The method may comprise a step for introducing an endoscope, e.g., an echoendoscope, e.g., a curvilinear or linear echoendoscope, into the patient, e.g., the GI tract of the patient (e.g., via the esophagus, stomach, or duodenum) to access the target site (e.g., pancreas). In some implementations, ultrasound imaging, e.g. real-time ultrasound imaging, may be used to visualize the target lesion (e.g., pancreatic cyst or pNET), confirm its location, confirm its size, confirm its characteristics, or any combination thereof.

The exemplary method may further comprise a step for inserting the apparatus (e.g., a multi-modality device according to various embodiments). Insertion may comprise feeding a multi-function apparatus, e.g., multi-function needle (capable of FNA/FNB, and ablation) described herein through the working channel of the endoscope, e.g., echoendoscope. The apparatus/treatment tool may be advanced, for example, under EUS guidance, to the target lesion and may be placed within or adjacent to the target lesion (e.g., pancreatic cyst or solid mass).

In some implementations, the method may comprise a step of acquiring a sample of the tissue or fluid, e.g., tissue or fluid from the target location. In one example, e.g., a method comprising use of fine needle aspiration, a needle may be inserted into the target lesion, e.g., pancreatic cyst. In some examples, using the apparatus, suction can be applied. Suction may be applied via an automated suction mechanism, such as an automated suction mechanism facilitated by activation of an activation mechanism present on an apparatus handle (described herein) or, e.g., by manual application of suction via, e.g., a syringe, to aspirate fluid, cellular material, or both through the needle lumen into a collection container/system. The collection system can be, for example, any suitable collection system such as a syringe that may, for example, be attached to the handle of the exemplary apparatus. The needle may be maintained in place after aspiration to proceed directly to ablation once the sample is collected. In certain aspects, the exemplary method comprises no step of removing the needle.

In an alternative example, an exemplary method may comprise using fine needle biopsy. In such a method, a needle may be inserted into the target lesion (e.g., pNET or solid mass). A biopsy may be performed by advancing and retracting the needle to acquire a core tissue sample. In some examples, the tissue sample may be captured at the needle tip or within a lumen present in the needle. According to some examples, the needle may be withdrawn through the endoscope, e.g., echoendoscope, to retrieve the tissue sample. In some implementations, the method can comprise flushing or ejecting the tissue sample from the needle tip into a sample collection system, e.g., a container containing preservative (e.g., formalin or saline) for analysis, e.g., histopathological analysis. The needle may be reintroduced through the endoscope, e.g., echoendoscope, working channel to the target lesion under EUS guidance for, e.g., subsequent ablation.

In some implementations, the exemplified method may comprise a step of ablation of the target lesion. For example, with the combination treatment tool (e.g., electrode needle) positioned within or adjacent to the target lesion, e.g., post-FNA or post-FNB reinsertion, pulsed electric fields, for example, nanosecond pulsed electric fields may be delivered through bipolar electrodes integrated into the treatment tool (electrode needle). The nanosecond pulsed electric fields may induce non-thermal ablation of the target tissue or cyst wall.

In certain examples, method(s) may comprise monitoring the ablation process in real-time using imaging technologies, such as, e.g., EUS imaging.

In some examples, method(s) can comprise an optional step of post-ablation assessment. The combination tool/needle may be removed from the target lesion through the endoscope, e.g., echoendoscope, working channel. A method may comprise optionally performing one or more additional assessment steps, such as, e.g., use of contrast-enhanced ultrasound (CEUS), by, e.g., injecting a microbubble contrast agent through the echoendoscope or a separate needle device, for example, to evaluate the ablation zone. In certain examples. EUS and CEUS imaging are used to assess the cellular and structural effects of ablation, confirming the extent of tissue destruction or cyst collapse, identifying any residual viable tissue, or both.

According to further implementations, a method may optionally comprise, upon completion of the procedure, removing the endoscope, e.g., echoendoscope, from the patient, and disposing of it per standard medical protocols. Collected tissue/fluid samples collected during the procedure may be processed and analyzed, during, after, or both during and after the procedure. Such analysis can comprise, e.g., cytologic analysis, histopathologic analysis, molecular testing, microscopic analysis, etc.

Upon completion of the procedure according to the present disclosure, a healthcare professional may monitor the patient for post-procedure complications (such as, e.g., bleeding, infection, or pancreatitis) according to standard protocols, e.g., standard EUS protocol.

Method comprising use of combination apparatuses described herein provide for numerous advantageous, including eliminating the need for using multiple instruments to accomplish sample collection, fluid/drug infusion and ablation, reducing procedure time, minimizing patient risk associated with repeated needle insertions, just to new a few. For example, reducing the number of needle passes/punctures can increase risk of pancreatitis, a risk reduced by method(s) and use of apparatuses disclosed herein. In addition, when implemented with the use of real-time guidance systems, such as, e.g., EUS, provides continuous visualization for precise needle placement, sample acquisition, and ablation. Further, in implementations using nanosecond pulsed electric fields, it enables targeted tissue destruction with minimal collateral damage. Such technology is suitable for sensitive target areas such as, e.g., the pancreas, ducts, etc. Various apparatuses according to present disclosure may support both FNA (fluid/cellular aspiration) and FNB (core tissue biopsy), thereby accommodating diverse lesion types.

Methods described herein in various examples can comprise additional steps, eliminate unnecessary steps, or perform steps in a different order according to the needs of the procedure at hand. For example, method(s) addressing cystic lesions may prioritize fluid aspiration followed by ablation of the cyst wall to prevent recurrence. In methods directed to the treatment of solid masses (e.g., pNET), FNB may be preferred to obtain sufficient tissue for diagnosis, followed by ablation of the tumor mass.

In some examples, method(s)/procedure(s) provided above can comprise an optional step for infusing a material, such as saline, drug, medication, or other material as described herein in addition to the step(s) for ablating and sample collecting.

According to other embodiments and implementations, the methods disclosed herein may provide for tissue ablation combined with infusion of fluids and other materials. According to some examples, apparatuses of the present disclosure may be used in procedures, e.g., procedures with or without EUS-guidance, for ablating a target location as well as delivering a material to the target location, such as infusion of a drug or other medication. In some examples, the apparatus is a single, multi-function needle device configured for both infusion and ablation, including nanosecond (ns) pulsed field ablation. In some examples, the tip region (electrode/needle) of such an apparatus may enable both infusion and therapeutic ablation during a single procedure, without requiring device exchange.

The method may comprise a step for introducing an endoscope, e.g., an echoendoscope, e.g., a curvilinear or linear echoendoscope, into the patient, e.g., the GI tract of the patient (e.g., via the esophagus, stomach, or duodenum) to access the target site (e.g., pancreas). Other anatomical sites are envisioned and exemplified elsewhere herein. In some implementations, ultrasound imaging, e.g. real-time ultrasound imaging, may be used to visualize the target lesion (e.g., pancreatic cyst or pNET), confirm its location, confirm its size, confirm its characteristics, or any combination thereof. Again, target tissues may vary.

According to some implementations, the method may further comprise a step for delivering the apparatus (e.g., a multi-modality device according to various embodiments) to a target tissue. Delivering may comprise feeding the apparatus through the working channel of the endoscope, e.g., echoendoscope. The apparatus/treatment tool may be advanced, for example, under EUS guidance, to the target lesion and may be placed within or adjacent to the target tissue/lesion (e.g., pancreatic cyst or solid mass).

In some implementations, the method may comprise a step of infusing a material to the target location, for example, before or after performing an ablation of the target tissue. In one example, e.g., a method comprises a step for delivering the multi-function needle to the target lesion and, upon placement, infusing a fluid into the target tissue, such as, e.g., a therapeutic agent such as a drug, medication, etc., for example, by application of a positive pressure. Infusion may be accomplished manually, e.g., by manual operation of a syringe or may be assisted by or completed via automation, such as by activation of an automated or semi-automated dispensation mechanism. Infusion may be activated in various examples by activation of a mechanism present, for example, on an apparatus handle, such as an infusion activation mechanism. The step of ablation may be performed in monopolar or bipolar manner (using various apparatuses of the present disclosure), including using a distal needle as an ablation electrode.

As mentioned above, any of the apparatuses described herein may be implemented in robotic apparatus that may be used to position and/or control the electrodes during a treatment. For example, a robotic apparatus may include a movable (robotic) arm to which the treatment apparatus or tool is coupled. Various motors and other movement devices may be incorporated to enable fine movements of an operating tip of the apparatus in multiple directions. The robotic apparatus and/or system may further include at least one image acquisition device (and preferably two for stereo vision, or more) which may be mounted in a fixed position or coupled (directly or indirectly) to a robotic arm or other controllable motion device. In some embodiments, the image acquisition device(s) may be incorporated into the apparatus of the present disclosure.

Embodiments of the methods and, also or alternatively feature(s) of apparatuses of the present disclosure may be implemented or controlled by using computer software, firmware or hardware. Various programming languages and operating apparatus may be used to implement the present disclosure. The program that runs the method and apparatus may include a separate program code including a set of instructions for performing a desired operation or may include a plurality of modules that perform such sub-operations of an operation or may be part of a single module of a larger program providing the operation. The modular construction facilitates adding, deleting, updating and/or amending the modules therein and/or features within the modules.

In some embodiments, a user may select a particular method or embodiment of this application, and the processor will run a program or algorithm associated with the selected method. In certain embodiments, various types of position sensors may be used. For example, in certain embodiments, a non-optical encoder may be used where a voltage level or polarity may be adjusted as a function of encoder signal feedback to achieve a desired angle, speed, or force.

Certain embodiments may relate to a machine-readable medium (e.g., computer readable media) or computer program products that include program instructions and/or data (including data structures) for performing various computer-implemented operations. A machine-readable medium may be used to store software and data which causes the apparatus to perform methods of the present disclosure. The above-mentioned machine-readable medium may include any suitable medium capable of storing and transmitting information in a form accessible by processing device, for example, a computer. Some examples of the machine-readable medium include, but not limited to, magnetic disc storage such as hard disks, floppy disks, magnetic tapes. It may also include a flash memory device, optical storage, random access memory, etc. The data and program instructions may also be embodied on a carrier wave or other transport medium. Examples of program instructions include both machine code, such as produced by a compiler, and files containing higher level code that may be executed using an interpreter.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform or control performing of any of the steps, including but not limited to: displaying, communicating with the user, analyzing, modifying parameters (including timing, frequency, intensity, etc.), determining, alerting, or the like. In some exemplary embodiments hardware may be used in combination with software instructions to implement the present disclosure.

When a feature or element is herein referred to as being “on” another feature or element, it can be directly on the other feature or element, or intervening features and/or elements may also be present. In contrast, when a feature or element is referred to as being “directly on” another feature or element, there are no intervening features or elements present. It will also be understood that, when a feature or element is referred to as being “mounted”, “connected”, “attached” or “coupled” to another feature or element, it can be directly mounted, connected, attached or coupled to the other feature or element or intervening features or elements may be present. In contrast, when a feature or element is referred to as being “directly mounted”, “directly connected”, “directly attached” or “directly coupled” to another feature or element, there are no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present apparatuses and methods.

The terms “comprises” and/or “comprising,” when used in this specification (including the claims), specify the presence of stated features, steps, operations, elements, and/or components, but do not preclude the presence or addition of one or more other features, steps, operations, elements, components, and/or groups thereof. Unless the context requires otherwise, “comprise”, and variations such as “comprises” and “comprising,” means various components may be co-jointly employed in the methods and articles (e.g., compositions and apparatuses including device and methods). For example, the term “comprising” will be understood to imply the inclusion of any stated elements or steps but not the exclusion of any other elements or steps.

Any of the apparatuses and methods described herein may include all or a sub-set of the components and/or steps, and these components or steps may be either non-exclusive (e.g., may include additional components and/or steps) or in some variations may be exclusive, and therefore may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about”, “substantially”, or “approximately,” even if the term does not expressly appear. The phrase “about”, “substantially”, or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and apparatus embodiments may be included in some embodiments and not in others. Therefore, the foregoing description is provided primarily for exemplary purposes and should not be interpreted to limit the scope of the apparatuses and methods as it is set forth in the claims.

Various embodiments may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Further, all publications and patent applications mentioned in this specification are herein incorporated by reference in their entirety to the same extent as if each individual publication or patent application was specifically and individually indicated to be incorporated by reference.

What is claimed is:

1. An apparatus for delivering a pulsed electric field, the apparatus comprising:
an elongate shaft; and
a tip region extending distally from the elongate shaft and having an outer diameter, the tip region comprising:
a first electrode,
a second electrode distal to the first electrode and having a voltage differential relative to the first electrode;
a spacer between the first electrode and the second electrode; and
a support member concentrically arranged relative to the first and second electrodes,
wherein the support member extends along at least a portion of a distance between the first and the second electrodes and is electrically separated in an axial direction from at least one of the first electrode and any components electrically coupled to the first electrode, or the second electrode and any components electrically coupled to the second electrode.

2. The apparatus of claim 1, wherein the first electrode is electrically coupled to provide a first polarity, and the second electrode is electrically coupled to provide a polarity opposite that of the first electrode.

3. The apparatus of claim 1, wherein the support member overlaps with at least one of the first and the second electrodes along a longitudinal axis of the tip region.

4. The apparatus of claim 2, wherein the overlap along the longitudinal axis is approximately between 0.75 and 3 times a diameter of the support member, in particular approximately 1 time the diameter of the support member.

5. The apparatus of claim 1, wherein the apparatus further comprises at least one insulative layer concentrically arranged along a portion of the tip region between the support member and the electrically separated first or second electrode.

6. The apparatus of claim 5, wherein the electrical separation of the support member from one of the first or second electrodes in the axial direction is at least partially based on the voltage differential between the first and second electrodes and/or a dielectric strength of the insulative layer.

7. The apparatus of claim 1, wherein the spacer is conductive or insulative.

8. The apparatus of claim 1, wherein the apparatus is configured to deliver high voltage non-thermal electric pulses.

9. The apparatus of claim 8, wherein the non-thermal electric pulses are nanosecond electric pulses.

10. The apparatus of claim 1, wherein the tip region further comprises an aspiration or infusion lumen configured to deliver a fluid to a target tissue and/or to provide negative pressure/suction to a target tissue.

11. The apparatus of claim 10, wherein the tip region comprises one or more ports or outlets within a distal tip of the second distal electrode, or along a length of any or all of the first electrode, the second electrode, or the spacer.

12. An apparatus for delivering a pulsed electric field, the apparatus comprising:
an elongate shaft; and
a tip region extending distally from the elongate shaft and having an outer diameter, the tip region comprising:
a first electrode,
a second electrode distal to the first electrode;
a spacer between the first electrode and the second electrode; and
a support member concentrically arranged relative to the first and second electrodes,
wherein the support member (1) physically overlaps with each of the proximal and distal electrodes along a longitudinal axis of the tip region; and (2) electrically separated radially and axially from at least one of the first electrode and any components electrically coupled to the first electrode, or the second electrode and any components electrically coupled to the second electrode.

13. The apparatus of claim 12, wherein the apparatus is configured to deliver high voltage microsecond or sub-microsecond electric pulses.

14. The apparatus of claim 12, wherein the tip region further comprises an aspiration or infusion lumen configured to deliver a fluid to a target tissue and/or to provide negative pressure/suction to a target tissue.

15. The apparatus of claim 14, the apparatus further comprising one or more ports or outlets of the aspiration or infusion lumen within a distal tip of the second distal electrode, and/or along a length of any or all of the first electrode, the second electrode, or the spacer.

16. The apparatus of claim 12, wherein the first electrode is configured to have a voltage differential relative to the second electrode and wherein the support member is electrically separated from the first electrode.

17. The apparatus of claim 16, the apparatus further comprises one or more insulative layer(s) and/or an adhering material concentrically arranged along a portion of the tip region between the support member and the electrically separated first electrode.

18. The apparatus of claim 17, wherein the support member is electrically separated from the first electrode by a distance in the axial direction that is at least partially based on the voltage differential between the first and second electrodes and/or a dielectric strength of the one or more insulative layer(s) and/or the adhering material.

19. A multi-modal apparatus for delivering a pulsed electric field, the apparatus comprising:
an elongate shaft; and
a tip region extending distally from the elongate shaft,
the tip region comprising:
a first electrode,
a second electrode distal to the first electrode;
a spacer between the first electrode and the second electrode; and
at least one aspiration or infusion lumen extending through at least a portion of the tip region,
wherein the apparatus is configured to deliver a bipolar pulsed electric field to a target tissue and to perform one or more of the following: (a) deliver a fluid from the tip region to the target tissue, (b) provide a negative pressure at the tip region to assure a proper contact with the target tissue and/or to facilitate collection of a sample from the target tissue, or (c) both (a) and (b).

20. The apparatus of claim 19, wherein delivering the fluid comprises delivering a solution, a medication, a drug, or therapeutic agent.

21. The apparatus of claim 20, wherein at least one of the at least one infusion or infusion lumens extends all the way to a distal tip of the distal electrode or terminates at one or more ports proximal to the distal tip of the distal electrode.

22. The apparatus of claim 19, wherein the tip region further comprises a support member concentrically arranged relative to the first and second electrodes, wherein the support member extends along at least a portion of a distance between the first and the second electrodes and is electrically separated at least in an axial direction from at least one of the first electrode and any components electrically coupled to the first electrode, or the second electrode and any components electrically coupled to the second electrode.
